(19) United States
(12) Patent Application Publication
(10) Pub. No.: US 2026/0166083 A1
(43) Pub. Date: Jun. 18, 2026

(54) METHODS AND COMPOSITIONS FOR CELL-SURFACE CO-LOCALIZATION OF CHIMERIC PROTEINS

(71) Applicant: Senti Biosciences, Inc., South San Francisco, CA (US)

(72) Inventors: Alba Gonzalez Junca, Burlingame, CA (US); Nicholas Frankel, San Francisco, CA (US); Maelig Goulwen Morvan, San Francisco, CA (US)

(21) Appl. No.: 19/126,923

(22) PCT Filed: Nov. 6, 2023
(86) PCT No.: PCT/US2023/078786

§ 371 Date: May 2, 2025

Related U.S. Application Data
(60) Provisional application No. 63/423,364, filed on Nov. 7, 2022.

(51) Int. Cl. A61K 35/17; A61K 40/15; A61K 40/31; A61K 40/42; C07K 14/705; C07K 14/725; C07K 16/28; C07K 16/30; C12N 5/0783

(52) U.S. Cl. CPC A61K 35/17; A61K 40/15; A61K 40/31; A61K 40/421; A61K 40/4266; C07K 14/7051; C07K 14/70517; C07K 14/70521; C07K 14/7056; C07K 16/2803; C07K 16/3007; C12N 5/0646; A61K 2239/13; A61K 2239/17; A61K 2239/21; A61K 2239/29; C07K 2317/31; C07K 2317/53; C07K 2319/02; C07K 2319/03; C12N 2510/00

(57) ABSTRACT

The present disclosure provides compositions and methods related to the co-localization of chimeric proteins on a cell surface. In particular, the present disclosure provides methods and compositions for facilitating the cell-surface co-localization of chimeric polypeptides using dimerization domains to promote their functional interaction.

### CROSS-REFERENCE TO RELATED APPLICATIONS

This application claims the benefit of U.S. Provisional Application No. 63/423,364, filed Nov. 7, 2022, the content of which is herein incorporated by reference in its entirety.

### FIELD

The present disclosure provides compositions and methods related to the co-localization of chimeric proteins on a cell surface. In particular, the present disclosure provides methods and compositions for facilitating the cell-surface co-localization of chimeric polypeptides (e.g., chimeric antigen receptors) using dimerization domains to promote their functional interaction (e.g., inhibition of an activating signal).

### SEQUENCE LISTING STATEMENT

The contents of the electronic sequence listing titled SENTI_41244_601_ST26.xml (Size: 253,510 bytes; and Date of Creation: Nov. 6, 2023) is herein incorporated by reference in its entirety.

### BACKGROUND

The immune system is essential for detecting and combating human cancer. The majority of transformed cells are quickly detected by immune sentinels and destroyed through the activation of immune effector cells, including T cells, NK cells, NKT cells, neutrophils and cells of the monocyte/macrophage lineage. Accordingly, cancer can be considered an immunological disorder, a failure of immune system to mount the necessary anti-tumor response to durably suppress and eliminate the disease. In order to more effectively combat cancer, certain immunotherapy interventions developed over the last few decades have specifically focused on enhancing one or more aspects of immune effector cells and engineering these cells to target specific tumor antigens (e.g., using chimeric antigen receptors).

Chimeric antigen receptors (CARs) enable targeted in vivo activation of immune effector cells. These recombinant membrane receptors generally have an antigen-binding domain and one or more signaling domains to allow the effector cells to recognize a specific protein antigen on tumor cells and induce activation and signaling pathways. Recent results of clinical trials with chimeric receptor-expressing T cells have provided compelling support of their utility as agents for cancer immunotherapy. However, despite these promising results, a number of side effects associated the CAR T-cell therapeutics have been identified, raising significant safety concerns. One side effect is “on-target/off-tissue” adverse events from TCR and CAR engineered effector cells, which occurs when a CAR effector cell binds to its ligand outside of the target tumor tissue and induces an immune response. Therefore, methods are needed for effectively targeting and treating a tumor without damaging normal cells that express the same target antigen.

### SUMMARY

Embodiments of the present disclosure include a chimeric protein system. In accordance with these embodiments, the chimeric protein system includes a first polypeptide comprising a first antigen binding domain, a first transmembrane domain, and a first dimerization domain; and a second polypeptide comprising a second antigen binding domain, a second transmembrane domain, and a second dimerization domain. In some embodiments, the first dimerization domain is capable of binding to the second dimerization domain. The chimeric protein system comprising first and second polypeptides provides, in some embodiments, pairing of chimeric antigen receptors (e.g., co-localizing of the receptor pairs) at the cellular surface (e.g., via one or more dimerization domains).

In some embodiments, the first dimerization domain or the second dimerization domain is selected from the group consisting of CD94, NKG2A, and NKG2C. In some embodiments, the first dimerization domain comprises a dimerization domain from CD94, and the second dimerization domain comprises a dimerization domain from NKG2A or a dimerization domain from NKG2C. In some embodiments, the first dimerization domain comprises a dimerization domain from NKG2A or a dimerization domain from NKG2C, and the second dimerization domain comprises a dimerization domain from CD94. The disclosure is not limited to any particular dimerization domain. Indeed, any one or more protein domains and/or amino acid sequences useful in forming a complex between a first and second polypeptide disclosed herein (e.g., to form a pair of chimeric antigen receptors) may be used. In some embodiments, the protein domain and/or amino acid sequence used to form a complex is a leucine zipper domain. In some embodiments, a first protein domain and/or amino acid sequence is used to form a complex with a second protein domain and/or amino acid sequence to form a heterodimer (e.g., via disulfide bonding). Non-limiting examples of protein domains and/or amino acid sequences useful in forming a complex include, but are not limited to, CD94, NKG2A, NKG2C, R34(GS)₃, E34-I(GS)₃, E34-V(GS)₃, E34-N(GS)₃, CD94-rev, NKG2A-rev, NKG2C-rev, (GS3)₃ R34, (GS3)₃ E34-I, (GS3)₃ E34-V, and (GS3)₃ E34-N.

In some embodiments, the first dimerization domain comprises a sequence selected from the group consisting of SEQ ID NOs: 70, 72, 74, 76, 78, 80, 82, 84, 86, 88, and 90. In some embodiments, the second dimerization domain comprises a sequence selected from the group consisting of SEQ ID NO: 70, 72, 74, 76, 78, 80, 82, 84, 86, 88, and 90.

In some embodiments, the first polypeptide is a chimeric antigen receptor. In some embodiments, the first polypeptide is an activating chimeric antigen receptor. In some embodiments, the first polypeptide is an inhibitory chimeric antigen receptor.

In some embodiments, the second polypeptide is a chimeric antigen receptor. In some embodiments, the second polypeptide is an activating chimeric antigen receptor. In some embodiments, the second polypeptide is an inhibitory chimeric antigen receptor.

In some embodiments, the antigen binding domain comprises a F(ab) fragment, a F(ab′) fragment, a single chain variable fragment (scFv), a single domain antibody, a diabody, a VHH fragment, or a synthetic epitope. In some embodiments, the antigen binding domain binds an antigen expressed on a cancer cell. In some embodiments, the cancer comprises glioblastoma, neuroblastoma, breast cancer, colorectal cancer, prostate cancer, bladder cancer, liver cancer, lung cancer, pancreatic cancer, ovarian cancer, gastric cancer, endometrial cancer, cervical cancer, leukemia (e.g., lymphatic, lymphocytic, lymphoblastic myelogenous or granulocytic leukemia), lymphoma, or myeloma.

In some embodiments, the antigen binding domain is specific for carcinoembryonic antigen (CEA), Mesothelin, Ax1, GPC3, FLT3, CD33, TROP2, MUC1, MUC16, IL13Ra, ErbB2 (HER2/neu), epithelial cell adhesion molecule (EpCAM), epidermal growth factor receptor (EGFR), EGFR variant III (EGFRvIII), CD19, CD20, CD30, CD40, disialoganglioside GD2, ductal-epithelial mucine, gp36, TAG-72, glycosphingolipids, glioma-associated antigen, alphafetoprotein (AFP), lectin-reactive AFP, thyroglobulin, RAGE 1, MN-CA IX, human telomerase reverse transcriptase, RU1, RU2 (AS), intestinal carboxyl esterase, mut hsp70-2, M-CSF, prostate, prostate specific antigen (PSA), PAP, NY-ESO-1, LAGA-1a, p53, prostein, PSMA, prostate-carcinoma tumor antigen-1 (PCTA-1), MAGE, ELF2M, neutrophil elastase, ephrin B2, CD22, insulin growth factor (IGF)-1, IGF-11, IGF-1 receptor, NKG2D, BCMA (CD269, TNFRSF 17), Claudin18.2, B7-H3, or Ror1.

In some embodiments, the transmembrane domain is selected from the group consisting of: a LAX transmembrane domain, a CD25 transmembrane domain, a CD7 transmembrane domain, a LAT transmembrane domain, a transmembrane domain from a LAT mutant, a BTLA transmembrane domain, a CDS transmembrane domain, a CD28 transmembrane domain, a CD3zeta transmembrane domain, a CD4 transmembrane domain, a 4-IBB transmembrane domain, an OX40 transmembrane domain, an ICOS transmembrane domain, a 2B4 transmembrane domain, a PD-1 transmembrane domain, a CTLA4 transmembrane domain, a BTLA transmembrane domain, a TIM3 transmembrane domain, a LIR1 transmembrane domain, an NKG2A transmembrane domain, a TIGIT transmembrane domain, and a LAG3 transmembrane domain, a LAIR1 transmembrane domain, a GRB-2 transmembrane domain, a Dok-1 transmembrane domain, a Dok-2 transmembrane domain, a SLAP1 transmembrane domain, a SLAP2 transmembrane domain, a CD200R transmembrane domain, an SIRPa transmembrane domain, an HAVR transmembrane domain, a GITR transmembrane domain, a PD-L1 transmembrane domain, a KIR2DL1 transmembrane domain, a KIR2DL2 transmembrane domain, a KIR2DL3 transmembrane domain, a KIR3DL1 transmembrane domain, a KIR3DL2 transmembrane domain, a CD94 transmembrane domain, a KLRG-1 transmembrane domain, a PAG transmembrane domain, a CD45 transmembrane domain, and a CEACAM1 transmembrane domain.

In some embodiments, the first polypeptide and/or the second polypeptide comprises one or more intracellular signaling domains. In some embodiments, the one or more intracellular signaling domains are selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CDS intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.

In some embodiments, the one or more intracellular signaling domains comprises a co-stimulatory domain selected from the group consisting of: a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CDS intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.

In some embodiments, the first polypeptide and/or the second polypeptide comprises a hinge domain located between the antigen binding domain and the transmembrane domain.

In some embodiments, the first polypeptide and/or the second polypeptide comprises one or more linkers. In some embodiments, the one or more linkers comprise a GSG linker, a Whitlow linker, an eGK linker, or any derivative thereof.

Embodiments of the present disclosure also include an engineered polynucleotide encoding any of the first polynucleotides and/or the second polynucleotides described herein.

Embodiments of the present disclosure also include an expression vector comprising any of the engineered polynucleotides described herein.

Embodiments of the present disclosure also include an engineered cell comprising any of the engineered polynucleotides of the present disclosure, the vectors, or the first and/or second polypeptides described herein.

In some embodiments, the cell is selected from the group consisting of: a T cell, a CDS+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

In some embodiments, the cell is engineered to express an effector molecule. In some embodiments, the cell is a Natural Killer (NK) cell. In some embodiments, the cell is autologous. In some embodiments, the cell is allogeneic.

Embodiments of the present disclosure also include a pharmaceutical composition comprising any of the engineered cells described herein, and a pharmaceutically acceptable carrier and/or a pharmaceutically acceptable excipient.

Embodiments of the present disclosure also include a method of treating a subject having cancer, the method comprising administering to the subject a therapeutically effective dose of any of the compositions described herein, or any of the cells described herein, to the subject. In some embodiments, the cancer comprises glioblastoma, neuroblastoma, breast cancer, colorectal cancer, prostate cancer, bladder cancer, liver cancer, lung cancer, pancreatic cancer, ovarian cancer, gastric cancer, endometrial cancer, cervical cancer, leukemia (e.g., lymphatic, lymphocytic, lymphoblastic myelogenous or granulocytic leukemia), lymphoma, or myeloma.

Embodiments of the present disclosure also include a method of enhancing immune cell-mediated killing of a cancer cell in a subject in need thereof, the method comprising administering to the subject a therapeutically effective dose of any of the compositions described herein, or any of the cells described herein, to the subject. In some embodiments, the cancer comprises a solid tumor. In some embodiments, the cancer comprises glioblastoma, neuroblastoma, breast cancer, colorectal cancer, prostate cancer, bladder cancer, liver cancer, lung cancer, pancreatic cancer, ovarian cancer, gastric cancer, endometrial cancer, cervical cancer, leukemia (e.g., lymphatic, lymphocytic, lymphoblastic myelogenous or granulocytic leukemia), lymphoma, or myeloma.

Embodiments of the present disclosure also include a method of reducing off-target killing of a healthy cell in a subject, the method comprising administering a therapeutically effective dose of any of the compositions described herein, or any of the cells described herein, to the subject, wherein the subject has been diagnosed with cancer. In some embodiments, the cancer comprises a solid tumor. In some embodiments, the cancer comprises glioblastoma, neuroblastoma, breast cancer, colorectal cancer, prostate cancer, bladder cancer, liver cancer, lung cancer, pancreatic cancer, ovarian cancer, gastric cancer, endometrial cancer, cervical cancer, leukemia (e.g., lymphatic, lymphocytic, lymphoblastic myelogenous or granulocytic leukemia), lymphoma, or myeloma.

Other aspects and embodiments of the disclosure will be apparent in light of the following detailed description.

### BRIEF DESCRIPTION OF THE DRAWINGS

FIGS. 1A-1E: Representative data demonstrating successful transduction of exemplary aCAR/iCAR combinations with dimerization domains (FIG. 1A) in NK cells. Representative data from target cell killing assays when the target cells express VSIG2 are provided; the data is expressed as normalized E:T ratio (FIGS. 1B-1C) and as a percent of target cells alone (FIGS. 1D-1E).

FIGS. 2A-2C: Representative data from experiments conducted to establish a baseline level of fluorescence per well based on the percent of target cells alone, normalized to non-dimerizing aCAR/iCAR (FIG. 2A), NKG2C dimerization domain containing CAR (FIG. 2B), and CD94 dimerization domain containing CAR (FIG. 2C).

FIGS. 3A-3C: Representative data from killing assays conducted over three rounds of killing for all the indicated conditions. Target cells with VSIG2 are represented by hollowed out shapes, whereas target cells without VSIG2 are represented by solid shapes; and each color reflects the use of the same NK effector cells (with or without VSIG2-expressing target cells). Data for the first round is shown in FIG. 3A; data for the second round is shown in FIG. 3B; and data for the third round is shown in FIG. 3C.

FIGS. 4A-4C: Representative data from killing assays conducted over three rounds of killing for a subset of the test conditions involving aCD94.

FIGS. 5A-5C: Representative data from killing assays conducted over three rounds of killing for a subset of the test conditions involving aNKG2C.

FIGS. 6A-6C: Representative control data from killing assays. FIG. 6A provides data for non-dimerizing CAR and no virus controls; FIG. 6B provides data for single transduction controls; and FIG. 6C provides data for non-dimerizing CAR conditions (second and third rounds shown).

FIGS. 7A-7C: Representative data from killing assays conducted to compare an aCAR with CD94 dimerization domain+an iCAR with NKG2C dimerization domain pairings with non-dimerizing CAR and no virus controls. Data from FIG. 7A is from two rounds of killing, and data from FIG. 7B is from three rounds of killing. FIG. 7C expresses the data from FIG. 7B in bar graph form.

### DETAILED DESCRIPTION

The present disclosure provides compositions and methods related to the co-localization of chimeric proteins on a cell surface. In particular, the present disclosure provides methods and compositions for facilitating the cell-surface co-localization of chimeric polypeptides (e.g., chimeric antigen receptors) using dimerization domains to promote their functional interaction (e.g., inhibition of an activating signal).

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the present disclosure may be readily combined, without departing from the scope or spirit of the embodiments provided herein. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +/−20% or +/−10%, more preferably +/−5%, even more preferably +/−1%, and still more preferably +/−0.1% from the specified value, as such variations are appropriate to perform the disclosed methods.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies may be multimers of individual immunoglobulin molecules. Antibodies described in the present disclosure may exist in a variety of forms including, for example, polyclonal antibodies, monoclonal antibodies, Fv, single chain variable fragments (scFv), Fab and F(ab)₂ fragments, VHH fragments, diabodies, synthetic epitopes, single domain antibodies, human antibodies, and humanized antibodies (See, e.g., Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N. Y.; Houston et al., 1988, Proc. Natl. Acad. Sci. USA 85:5879-5883; Bird et al., 1988, Science 242:423-426).

The term “antibody fragment” refers to a portion of an intact antibody and preferably refers to the antigenic determining variable regions of an intact antibody. Examples of antibody fragments include, but are not limited to, Fab, Fab′, F(ab′) 2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. Kappa and lamda light chains refer to the two major antibody light chain isotypes.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

By the terms “synthetic antibody” and “recombinant antibody” are used interchangeably herein to mean an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “immunoglobulin” or “Ig,” as used herein refers to a class of proteins, which function as antibodies. Antibodies expressed by B cells are sometimes referred to as the B cell receptor (BCR) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The terms “chimeric receptor,” “Chimeric Antigen Receptor” or alternatively a “CAR” refer to a recombinant polypeptide comprising at least an extracellular antigen binding moiety, a transmembrane domain, and an intracellular signaling domain (also referred to as a “cytoplasmic signaling domain”) comprising a functional signaling domain.

As used herein, the term “antigen binding domain” refers to a protein that binds to a target antigen. An antigen binding domain can include, but is not limited to a protein (e.g., an immunoglobulin chain or fragment thereof) comprising at least one immunoglobulin variable domain sequence. The term “antigen binding domain” encompasses antibodies and antibody fragments. In some embodiments, an antigen binding domain is a multispecific antibody molecule (e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope). In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, an antigen binding domain can refer an effector protein, in which the effector protein can bind to its cognate protein partner, such as a receptor.

As used herein, the terms “extracellular domain” or “ECD” refer to a polypeptide or combinations of polypeptides that form the external portion of a larger polypeptide (e.g., chimeric antigen receptor) that extends outside the outer membrane of the cell or organelle in which it is expressed. In some embodiments, an extracellular domain includes more than one domain. In some embodiments, an extracellular domain includes distinct domains that are capable of interacting with different ligands. In other aspects, an extracellular domain includes distinct domains that are capable of being cleaved by a protease. In still other aspects, an extracellular domain is linked to other domains (e.g., transmembrane domain) to form a larger polypeptide (e.g., chimeric antigen receptor). In some embodiments, the extracellular domain includes a hinge region that links the extracellular domain to a transmembrane domain.

As used herein, the terms “transmembrane domain,” “TM,” or “TMD” refer to a polypeptide or combinations of polypeptides that traverse the membrane of a cell or organelle in which it is expressed. In some embodiments, a transmembrane domain is linked to an extracellular domain and/or an intracellular domain to form a larger polypeptide (e.g., chimeric antigen receptor) capable of transmitting an external signal into an intracellular response. In some embodiments, transmembrane domains are made of predominantly of nonpolar amino acid residues and may traverse the bilayer once (e.g., single pass) or several times (multi-chain). In some embodiments, the transmembrane domain single-pass transmembrane protein includes three domains, the extracellular domain, the transmembrane domain, and the intracellular domain. In some embodiments, the transmembrane domain forms an alpha helix and is inserted into a membrane bilayer.

As used herein, the terms “intracellular signaling domain,” “cytoplasmic domain,” or “ICD” refer to a polypeptide or combinations of polypeptides that are positioned within the lumen of a cell or organelle in which it is expressed. In some embodiments, an intracellular signaling domain is linked to an extracellular domain via a transmembrane domain to form a larger polypeptide (e.g., chimeric antigen receptor) capable of transmitting an external signal into an intracellular response. In some embodiments, the intracellular signaling domain includes a single signaling domain capable providing an intracellular activation signal upon binding of a ligand to the extracellular domain to which it is linked. In other aspects, the intracellular signaling domain includes more than one signaling domain capable providing one or more intracellular activation signal(s) upon binding of a ligand to the extracellular domain to which it is linked. In some embodiments, the intracellular signaling domain includes a primary intracellular signaling domain as well as a co-stimulatory domain.

As used herein, the term “autologous” is meant to refer to any material derived from the same individual to which it is later to be re-introduced into the individual. “Allogeneic” refers to a graft derived from a different animal of the same species. “Xenogeneic” refers to a graft derived from an animal of a different species.

The term “therapeutic” as used herein means a treatment and/or prophylaxis. A therapeutic effect is obtained by suppression, remission, or eradication of a disease state.

The term “effective amount” or “therapeutically effective amount” are used interchangeably herein, and refer to an amount of a compound, formulation, material, or composition, as described herein effective to achieve a particular biological result. For example, a “therapeutically effective amount” may be the amount of a compound or composition that will elicit the biological or medical response of a tissue, system, or subject that is being sought by the researcher, veterinarian, medical doctor or other clinician. A therapeutically effective amount includes that amount of a compound or composition that, when administered, is sufficient to prevent development of, or alleviate to some extent, one or more of the signs or symptoms of a disorder or disease being treated. The therapeutically effective amount will vary depending on the compound or composition, the disease and its severity and the age, weight, etc., of the subject to be treated.

To “treat” a disease as the term is used herein, means to reduce the frequency or severity of at least one sign or symptom of a disease or disorder experienced by a subject. For example, treat may refer to the reduction or amelioration of the progression, severity and/or duration of a proliferative disorder, or the amelioration of one or more symptoms (preferably, one or more discernible symptoms) of a proliferative disorder resulting from the administration of one or more therapies. In some embodiments, the terms “treat,” “treatment” and “treating” refer to the amelioration of at least one measurable physical parameter of a proliferative disorder, such as growth of a tumor, not necessarily discernible by the patient. In other aspects, the terms “treat,” “treatment,” and “treating” refer to the inhibition of the progression of a proliferative disorder, either physically by, e.g., stabilization of a discernible symptom, physiologically by, e.g., stabilization of a physical parameter, or both. In other aspects the terms “treat,” “treatment,” and “treating” refer to the reduction or stabilization of tumor size or cancerous cell count.

As used herein “endogenous” refers to any material from or produced inside an organism, cell, tissue or system. As used herein, the term “exogenous” refers to any material introduced from or produced outside an organism, cell, tissue, or system.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other aspects, the cells are not cultured in vitro.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein and are intended to include living organisms in which an immune response can be elicited (e.g., mammals). Examples of subjects include humans, dogs, cats, mice, rats, and transgenic species thereof.

By the term “specifically binds,” as used herein with respect to an antibody or extracellular target-binding moiety, is meant an antibody or extracellular target-binding moiety which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, e.g., of an immune effector cell, which enhances or promotes an immune attack of a target cell. For example, an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The terms “cancer antigen,” “cancer-associated antigen” or “tumor antigen” interchangeably refer to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 or CD123 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, antibodies (or antigen-binding portions thereof), and engineered immune cells of the present disclosure in prevention of the occurrence of tumor in the first place.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors for developing cancer. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the type and/or stage of cancer is not known. The term further includes people who previously had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, blood test, etc.

As used herein, the term “subject at risk for cancer” refers to a subject with one or more risk factors for developing a specific cancer. Risk factors include, but are not limited to, gender, age, genetic predisposition, environmental exposure, and previous incidents of cancer, preexisting non-cancer diseases, and lifestyle.

As used herein, the term “characterizing cancer in a subject” refers to the identification of one or more properties of a cancer sample in a subject, including but not limited to, the presence of benign, pre-cancerous or cancerous tissue and the stage of the cancer.

As used herein, the term “tumor microenvironment” refers to the cellular environment in which a tumor exists, including surrounding blood vessels, immune cells, fibroblasts, bone marrow-derived inflammatory cells, lymphocytes, signaling molecules and the extracellular matrix (ECM) (see, e.g., Pattabiraman, D. R. & Weinberg, R. A. Nature Reviews Drug Discovery 13, 497-512 (2014); Balkwill, F. R. et al. J Cell Sci 125, 5591-5596, 2012; and Li, H. et al. J Cell Biochem 101(4), 805-15, 2007).

“Refractory” as used herein refers to a disease, e.g., cancer, which does not respond to a treatment. In aspects, a refractory cancer can be resistant to a treatment before or at the beginning of the treatment. In other aspects, the refractory cancer can become resistant during a treatment. A refractory cancer is also called a resistant cancer.

“Relapsed” or “relapse” as used herein refers to the return or reappearance of a disease (e.g., cancer) or the signs and symptoms of a disease such as cancer after a period of improvement or responsiveness, e.g., after prior treatment of a therapy, e.g., cancer therapy. The initial period of responsiveness may involve the level of cancer cells falling below a certain threshold, e.g., below 20%, 1%, 10%, 5%, 4%, 3%, 2%, or 1%. The reappearance may involve the level of cancer cells rising above a certain threshold, e.g., above 20%, 1%, 10%, 5%, 4%, 3%, 2%, or 1%. For example, e.g., in the context of B-ALL, the reappearance may involve, e.g., a reappearance of blasts in the blood, bone marrow (>5%), or any extramedullary site, after a complete response. A complete response, in this context, may involve <5% BM blast. More generally, in one aspect, a response (e.g., complete response or partial response) can involve the absence of detectable MRD (minimal residual disease). In one aspect, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

The term “derived from” as used herein refers to a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3ζ molecule, the intracellular signaling domain retains sufficient CD3 ζ structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3ζ sequence and delete unwanted sequence, or impose mutations, to arrive at the intracellular signaling domain.

“Activation,” as used herein, refers to the state of an immune cell (e.g., NK cell or T cell) that has been sufficiently stimulated to induce a detectable change such as, but not limited to, detectable cellular proliferation or immune response. Activation can also be associated with induced cytokine production, and/or detectable effector functions. The term “activated NK cells” refers to, among other things, NK cells that are undergoing cell division and/or enhanced cytokine production and/or secretion.

As used herein, the term “immune response” refers to a response by the immune system of a subject. For example, immune responses include, but are not limited to, a detectable alteration (e.g., increase) in Toll receptor activation, lymphokine (e.g., cytokine (e.g., Th1, Th17, or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self-antigens from microorganisms (e.g., pathogens), or self-antigens recognized as foreign). The term “immune response” is meant to encompass all aspects of the capability of a subject's immune system to respond to antigens and/or immunogens (e.g., both the initial response to an immunogen as well as acquired (e.g., memory) responses that are a result of an adaptive immune response.

It should also be understood that, unless clearly indicated to the contrary, in any method claimed herein that includes more than one step or act, the order of the steps or acts of the method is not necessarily limited to the order in which the steps or acts of the method are recited.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Chimeric Protein Systems Comprising Dimerization Domains

Embodiments of the present disclosure include a chimeric protein system. In accordance with these embodiments, the chimeric protein system includes a first polypeptide comprising a first antigen binding domain, a first transmembrane domain, and a first dimerization domain; and a second polypeptide comprising a second antigen binding domain, a second transmembrane domain, and a second dimerization domain. In some embodiments, the first dimerization domain is capable of binding to the second dimerization domain.

In accordance with the various embodiments described herein, immune receptors (e.g., chimeric antigen receptors or CARs) with complementary or opposite functions (e.g., activating vs. inhibitory functions) can be engineered to include dimerization domains to facilitate their co-localization (e.g., at an immune synapse) in immune effector cells. In some embodiments, the dimerization domains form a heterodimer complex of immune receptors (e.g., receptors having complementary functions, receptors having non-complementary functions, or receptors having opposite functions). As described further herein, certain CARs, (e.g., activating CARs and inhibitory CARs), without being bound by theory, exhibit maximal efficacy when expressed in close proximity to each other in order to execute a desired function (e.g., inhibition of an activating signal). The data and results provided herein demonstrate that the inclusion of dimerization domains as a part of a CAR facilitates their co-localization and enhances their synergy and/or competing effects. In some embodiments, the dimerization domains can be situated in the extracellular or intracellular domain of the CAR.

The dimerization domains comprised within the first and second polypeptides of the chimeric protein systems of the present disclosure can be any dimerization domains that facilitate the co-localization of the first and the second polypeptides, as would be recognized by one of ordinary skill in the art based on the present disclosure. Indeed, the disclosure provides that the first and second polypeptides disclosed herein comprise any one or more protein domains and/or amino acid sequences (e.g., leucine zipper domain, and/or a first protein domain that binds via disulfide or other type of bonding to second protein domain) that form of a complex. In some embodiments, dimerization domains present in the first and second polypeptides facilitate pairing/complexing of chimeric antigen receptors. A non-limiting example of a first polypeptide and a second polypeptide that dimerize (form a complex and/or co-localize) to form a pair of chimeric antigen receptors use a dimerization domain from CD94 and a dimerization domain selected from NKG2A and NKG2C.

In some embodiments, the first dimerization domain or the second dimerization domain is selected from the group consisting of CD94, NKG2A, and NKG2C. In some embodiments, the first dimerization domain comprises a dimerization domain from CD94, and the second dimerization domain comprises a dimerization domain from NKG2A or a dimerization domain from NKG2C. In some embodiments, the first dimerization domain comprises a dimerization domain from NKG2A or a dimerization domain from NKG2C, and the second dimerization domain comprises a dimerization domain from CD94.

In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity with any of SEQ ID NOs: 70, 72, 74, 76, 78, 80, 82, 84, 86, 88, and 90. In some embodiments, the first dimerization domain comprises an amino acid sequence selected from the group consisting of SEQ ID NOs: 70, 72, 74, 76, 78, 80, 82, 84, 86, 88, and 90.

In some embodiments, the second dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity with any of SEQ ID NOs: 70, 72, 74, 76, 78, 80, 82, 84, 86, 88, and 90. In some embodiments, the second dimerization domain comprises a sequence selected from the group consisting of SEQ ID NO: 70, 72, 74, 76, 78, 80, 82, 84, 86, 88, and 90.

In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 70. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 72. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 74. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 76. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 78. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 80. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 82. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 84. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 86. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 88. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 90.

In some embodiments, the second dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 70. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 72. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 74. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 76. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 78. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 80. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 82. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 84. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 86. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 88. In some embodiments, the first dimerization domain comprises an amino acid sequence having at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity SEQ ID NO: 90.

In some embodiments, the first polypeptide and/or the second polypeptide of the chimeric protein system comprises an antigen binding domain, wherein the antigen binding domain is selected from the group consisting of a F(ab) fragment, a F(ab′) fragment, a single chain variable fragment (scFv), a single domain antibody, a diabody, a VHH fragment, and a synthetic epitope. In some embodiments, the scFv comprises a heavy chain variable domain (VH) and a light chain variable domain (VL). In some embodiments, the VH and VL are separated by a peptide linker. Generally, an scFv has a variable domain of light chain (VL) connected from its C-terminus to the N-terminal end of a variable domain of heavy chain (VH) by a polypeptide chain. Alternately, the scFv comprises of polypeptide chain where in the C-terminal end of the VH is connected to the N-terminal end of VL by a polypeptide chain. In some embodiments, the scFv comprises the structure VH-L-VL or VL-L-VH, wherein VH is the heavy chain variable domain, L is the peptide linker, and VL is the light chain variable domain. An sdAb is a molecule in which one variable domain of an antibody specifically binds to an antigen without the presence of the other variable domain. A F(ab) fragment contains the constant domain (CL) of the light chain and the first constant domain (CH1) of the heavy chain along with the variable domains VL and VH on the light and heavy chains respectively. F(ab′) fragments differ from Fab fragments by the addition of a few residues at the carboxy terminus of the heavy chain CH1 domain including one or more cysteines from the antibody hinge region. F(ab′)₂ fragments contain two Fab′ fragments joined, near the hinge region, by disulfide bonds. As used herein, a synthetic epitope refers to a non-naturally occurring epitope component residing within (e.g., that has been inserted into) the extracellular portion of the chimeric protein system (e.g., within the antigen binding domain, spacer, linker, or elsewhere). The disclosure is not limited to any particular synthetic epitope nor to how the synthetic epitope is utilized. For example, the synthetic epitope may be used to facilitate binding of an epitope-specific antibody to the chimeric protein system (e.g., for isolation and/or purification during manufacturing, or the depletion/removal of the protein system from a source).

### Chimeric Antigen Receptors

As detailed herein, certain aspects of the present disclosure relate to chimeric antigen receptors (CARs) that co-localize at a cell surface through forced association via one or more dimerization domains present within CAR polypeptide sequences. CARs containing one or more dimerization domains disclosed herein are not limited by specificity and/or function. In some embodiments, CARs containing one or more dimerization domains of the present disclosure comprise an extracellular antigen-binding domain fused to a transmembrane domain fused to one or more intracellular signaling domains. In some embodiments, a CAR comprises a spacer region or a hinge domain.

### Extracellular Antigen Binding Domain

In some embodiments, the first and/or second polypeptides of the chimeric protein systems of the present disclosure include an antigen binding domain that targets any tumor-associated antigen of interest. In some embodiments, the first and/or second polypeptides of the present disclosure are activating CARs (e.g., activating CARs that include a dimerization domain). In some embodiments, the antigen binding domain of the present disclosure binds an antigen expressed on the surface of a cancer cell. In some embodiments, the cancer cells is from a solid tumor. In some embodiments, the cancer includes, but is not limited to, glioblastoma, neuroblastoma, breast cancer, colorectal cancer, prostate cancer, bladder cancer, liver cancer, lung cancer, pancreatic cancer, ovarian cancer, gastric cancer, endometrial cancer, and cervical cancer.

In some embodiments, the antigen binding domain is specific for at least one of carcinoembryonic antigen (CEA), Mesothelin, Ax1, GPC3, FLT3, CD33, TROP2, MUC1, MUC16, IL13Ra, ErbB2 (HER2/neu), epithelial cell adhesion molecule (EpCAM), epidermal growth factor receptor (EGFR), EGFR variant III (EGFRvIII), CD19, CD20, CD30, CD40, disialoganglioside GD2, ductal-epithelial mucine, gp36, TAG-72, glycosphingolipids, glioma-associated antigen, alphafetoprotein (AFP), lectin-reactive AFP, thyroglobulin, RAGE 1, MN-CA IX, human telomerase reverse transcriptase, RU1, RU2 (AS), intestinal carboxyl esterase, mut hsp70-2, M-CSF, prostate, prostate specific antigen (PSA), PAP, NY-ESO-1, LAGA-1a, p53, prostein, PSMA, prostate-carcinoma tumor antigen-1 (PCTA-1), MAGE, ELF2M, neutrophil elastase, ephrin B2, CD22, insulin growth factor (IGF)-1, IGF-11, IGF-1 receptor, NKG2D, BCMA (CD269, TNFRSF 17), Claudin18.2, B7-H3, or Ror1.

In some embodiments, the first polypeptide of the chimeric protein system is a chimeric antigen receptor (CAR). In some embodiments, the first polypeptide is an activating chimeric antigen receptor (aCAR). In some embodiments, the first polypeptide is an inhibitory chimeric antigen receptor (iCAR). In some embodiments, the second polypeptide of the chimeric protein system is a chimeric antigen receptor (CAR). In some embodiments, the second polypeptide is an activating chimeric antigen receptor (aCAR). In some embodiments, the second polypeptide is an inhibitory chimeric antigen receptor (iCAR). In some embodiments, an activating chimeric antigen receptor (aCAR) is bivalent (e.g., it is specific for two target antigens). In some embodiments, the two target antigen are the same. In other embodiments, the two target antigens are different.

In accordance with the above embodiments, the first and/or second polypeptides (e.g., CARs) of the chimeric protein systems of the present disclosure include extracellular domains that comprise an antigen binding domain and a dimerization domain. In some embodiments, the dimerization domains facilitate co-localization (i.e., binding) of the first and second polypeptides on the cellular surface of an immune effector cell, such that the antigen binding domains of the first and second polypeptides can bind to their corresponding antigens. In some embodiments, for example, the first polypeptide can be an aCAR that comprises a dimerization domain and an antigen binding domain that is specific for an antigen on the surface of a cancer cell, and the second polypeptide can be an iCAR that comprises a dimerization domain and an antigen binding domain that is specific for an antigen on the surface of a non-cancerous cell (e.g., a healthy cell). Binding and co-localization of the first and second polypeptides on the cellular surface of an immune effector cell via their dimerization domains can result in the iCAR inhibiting activation of the aCAR (e.g., via prevention of intracellular signal activation of the aCAR) in the presence of a non-cancerous cell expressing both antigens. In this manner, the chimeric protein systems of the present disclosure provide a more specific and effective means for targeting a cancer cell and preventing or reducing off-target effects on non-cancerous cells.

### Transmembrane Domains

In some embodiments, the first polypeptide and/or the second polypeptide of the chimeric protein system comprises a transmembrane domain. The disclosure is not limited by the transmembrane domain utilized. In some embodiments, a CAR polypeptide sequence comprises a transmembrane domain from LIR1, LIR2, LIR3, LIR5, LIR8, IRTA1, IRTA2, IRTA4, LAIR1, BTLA, KIR2DL1, KIR2DL2, KIR2DL3, KIR2DL4, KIR2DL5, KIR3DL1, KIR3DL2, NKIR, TLT1, PD-1, CTLA4, TIM-3, LAG3, TIGIT, FCRH3, FCGR2B, SIGLEC-2, SIGLEC-6, SIGLEC-7, SIGLEC-12, SIGLEC-10, KLRG1, NKG2A, CD72, PECAM-1, CEACAM-1, PCDHGC3, PCDHGC5, CDH11, PTPRO, PTPRZ1, SLAMF1, SLAMF5, CD45, IMPG2, DSCAM, LIFR, ERMAP, IL1RAP, IL1RAPL2, CDH5, MPZL1, MPZ, MPIG6B, VSIG4, SLAP1, SLAP2, dok-1, Dok-2, GRB-2, CD200R, SIRPa, HAVR, GITR, PDL-1, and/or CD94.

TABLE 1
Exemplary transmembrane domain sequences.

| TM domain | Amino Acid Sequence | DNA Sequence |
|---|---|---|
| CD28 | FWVLVVVGGVLACYSLL | TTTTGGGTGCTGGTGGTGGTTGGTGGAGTCCTGGCT |
| | VTVAFIIFWV (SEQ ID | TGCTATAGCTTGCTAGTAACAGTGGCCTTTATTATTT |
| | NO: 123) | TCTGGGTG (SEQ ID NO: 124) |
| PAG | GPAGSLLGSGQMQITLW | GGTCCGGCTGGCTCTCTGCTCGGCAGTGGTCAGATG |
| | GSLAAVAIFFVITFLIFLCS | CAGATTACGTTGTGGGGCAGTTTGGCAGCCGTCGCA |
| | SCDREKKPR (SEQ ID NO: | ATCTTCTTTGTTATCACTTTTCTTATCTTTCTCTGTTC |
| | 125) | CTCATGTGACAGAGAGAAAAAGCCCCGA (SEQ ID |
| | | NO: 126) |
| mLAT | MEADALSPVGLGLLLLPF | ATGGAAGCCGATGCTCTGTCTCCTGTTGGCCTGGGA |
| | LVTLLAALCVRCRELPVS | CTGCTGCTCCTGCCTTTTCTGGTTACACTGCTGGCCG |
| | (SEQ ID NO: 127) | CTCTGTGTGTGCGGTGTAGAGAACTGCCAGTTAGT |
| | | (SEQ ID NO: 128) |
| mLAT(ca) | MEADALSPVGLGLLLLPF | ATGGAAGCCGATGCTCTGTCTCCTGTTGGCCTGGGA |
| | LVTLLAALAVRARELPVS | CTGCTGCTCCTGCCTTTTCTCGTTACACTGCTGGCCG |
| | (SEQ ID NO: 129) | CTCTGGCTGTGCGAGCTAGAGAACTGCCTGTGTCT |
| | | (SEQ ID NO: 130) |
| LAT(1-33) | EEAILVPCVLGLLLLPILA | GAGGAAGCAATCCTGGTGCCGTGTGTACTTGGTCTG |
| | MLMALCVHCHRLP (SEQ | CTTTTGTTGCCAATACTTGCGATGCTCATGGCTCTCT |
| | ID NO: 131) | GCGTACATTGCCATCGGCTTCCG (SEQ ID NO: 132) |
| LAX | IFSGFAGLLAILLVVAVFC | ATCTTCAGCGGCTTTGCCGGACTGCTGGCTATCCTG |
| | IL (SEQ ID NO: 133) | CTGGTTGTGGCCGTGTTCTGTATCCTT (SEQ ID NO: |
| | | 134) |
| CD3z | LCYLLDGILFIYGVILTAL | CTGTGCTACCTGCTGGACGGCATCCTGTTTATCTAC |
| | FL (SEQ ID NO: 135) | GGCGTGATCCTGACAGCCCTGTTCCTT (SEQ ID NO: |
| | | 136) |
| CD45 | ALIAFLAFLIIVTSIALLVV | GCCCTGATTGCCTTCCTGGCCTTTCTGATCATCGTGA |
| | L (SEQ ID NO: 137) | CCAGCATTGCCCTGCTGGTCGTGCTG (SEQ ID NO: |
| | | 138) |

In some embodiments, the transmembrane domain is selected from the group consisting of: a LAX transmembrane domain, a CD25 transmembrane domain, a CD7 transmembrane domain, a LAT transmembrane domain, a transmembrane domain from a LAT mutant, a BTLA transmembrane domain, a CDS transmembrane domain, a CD28 transmembrane domain, a CD3zeta transmembrane domain, a CD4 transmembrane domain, a 4-IBB transmembrane domain, an OX40 transmembrane domain, an ICOS transmembrane domain, a 2B4 transmembrane domain, a PD-1 transmembrane domain, a CTLA4 transmembrane domain, a BTLA transmembrane domain, a TIM3 transmembrane domain, a LIR1 transmembrane domain, an NKG2A transmembrane domain, a TIGIT transmembrane domain, and a LAG3 transmembrane domain, a LAIR1 transmembrane domain, a GRB-2 transmembrane domain, a Dok-1 transmembrane domain, a Dok-2 transmembrane domain, a SLAP1 transmembrane domain, a SLAP2 transmembrane domain, a CD200R transmembrane domain, an SIRPa transmembrane domain, an HAVR transmembrane domain, a GITR transmembrane domain, a PD-L1 transmembrane domain, a KIR2DL1 transmembrane domain, a KIR2DL2 transmembrane domain, a KIR2DL3 transmembrane domain, a KIR3DL1 transmembrane domain, a KIR3DL2 transmembrane domain, a CD94 transmembrane domain, a KLRG-1 transmembrane domain, a PAG transmembrane domain, a CD45 transmembrane domain, and a CEACAM1 transmembrane domain.

In some embodiments, the transmembrane domain of the first and/or second polypeptide of the chimeric protein systems of the present is derived from a CD8 polypeptide. Any suitable CD8 polypeptide may be used. Exemplary CD8 polypeptides include, without limitation, NCBI Reference Nos. NP_001139345 and AAA92533.1. In some embodiments, the transmembrane domain is derived from a CD28 polypeptide. Any suitable CD28 polypeptide may be used. Exemplary CD28 polypeptides include, without limitation, NCBI Reference Nos. NP_006130.1 and NP_031668.3. In some embodiments, the transmembrane domain is derived from a CD3-zeta polypeptide. Any suitable CD3-zeta polypeptide may be used. Exemplary CD3-zeta polypeptides include, without limitation, NCBI Reference Nos. NP_932170.1 and NP_001106862.1. In some embodiments, the transmembrane domain is derived from a CD4 polypeptide. Any suitable CD4 polypeptide may be used. Exemplary CD4 polypeptides include, without limitation, NCBI Reference Nos. NP_000607.1 and NP_038516.1. In some embodiments, the transmembrane domain is derived from a 4-1BB polypeptide. Any suitable 4-1BB polypeptide may be used. Exemplary 4-1BB polypeptides include, without limitation, NCBI Reference Nos. NP_001552.2 and NP_001070977.1. In some embodiments, the transmembrane domain is derived from an OX40 polypeptide. Any suitable OX40 polypeptide may be used. Exemplary OX40 polypeptides include, without limitation, NCBI Reference Nos. NP_003318.1 and NP_035789.1. In some embodiments, the transmembrane domain is derived from an ICOS polypeptide. Any suitable ICOS polypeptide may be used. Exemplary ICOS polypeptides include, without limitation, NCBI Reference Nos. NP_036224 and NP_059508. In some embodiments, the transmembrane domain is derived from a CTLA-4 polypeptide. Any suitable CTLA-4 polypeptide may be used. Exemplary CTLA-4 polypeptides include, without limitation, NCBI Reference Nos. NP_005205.2 and NP_033973.2. In some embodiments, the transmembrane domain is derived from a PD-1 polypeptide. Any suitable PD-1 polypeptide may be used. Exemplary PD-1 polypeptides include, without limitation, NCBI Reference Nos. NP_005009 and NP_032824. In some embodiments, the transmembrane domain is derived from a LAG-3 polypeptide. Any suitable LAG-3 polypeptide may be used. Exemplary LAG-3 polypeptides include, without limitation, NCBI Reference Nos. NP_002277.4 and NP_032505.1. In some embodiments, the transmembrane domain is derived from a 2B4 polypeptide. Any suitable 2B4 polypeptide may be used. Exemplary 2B4 polypeptides include, without limitation, NCBI Reference Nos. NP_057466.1 and NP_061199.2. In some embodiments, the transmembrane domain is derived from a BTLA polypeptide. Any suitable BTLA polypeptide may be used. Exemplary BTLA polypeptides include, without limitation, NCBI Reference Nos. NP_861445.4 and NP_001032808.2. Any suitable LIR-1 (LILRB1) polypeptide may be used. Exemplary LIR-1 (LILRB1) polypeptides include, without limitation, NCBI Reference Nos. NP_001075106.2 and NP_001075107.2.

In some embodiments, the transmembrane domain comprises a polypeptide comprising an amino acid sequence that is at least 85%, at least 90%, at least 91%, at least 92%, at least 93%, at least 94%, at least 95%, at least 96%, at least 97%, at least 98%, at least 99%, or 100% identical to the sequence of NCBI Reference No. NP_001139345, AAA92533.1, NP_006130.1, NP_031668.3, NP_932170.1, NP_001106862.1, NP_000607.1, NP_038516.1, NP_001552.2, NP_001070977.1, NP_003318.1, NP_035789.1, NP_036224, NP_059508, NP_005205.2, NP_033973.2, NP_005009, NP_032824, NP_002277.4, NP_032505.1, NP_057466.1, NP_061199.2, NP_861445.4, or NP_001032808.2, or fragments thereof. In some embodiments, the polypeptide may comprise one conservative amino acid substitution, up to two conservative amino acid substitutions, or up to three conservative amino acid substitutions. In some aspects, the polypeptide can have an amino acid sequence that is a consecutive portion of NCBI Reference No. NP_001139345, AAA92533.1, NP_006130.1, NP_031668.3, NP_932170.1, NP_001106862.1, NP_000607.1, NP_038516.1, NP_001552.2, NP_001070977.1, NP_003318.1, NP_035789.1, NP_036224, NP_059508, NP_005205.2, NP_033973.2, NP_005009, NP_032824, NP_002277.4, NP_032505.1, NP_057466.1, NP_061199.2, NP_861445.4, or NP_001032808.2 that is at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, or at least 240 amino acids in length.

Further examples of suitable polypeptides from which a transmembrane domain may be derived include, without limitation, the transmembrane region(s) of the alpha, beta or zeta chain of the T-cell receptor, CD27, CD3 epsilon, CD45, CD5, CD9, CD16, CD22, CD33, CD37, CD64, CD80, CD86, CD134, CD137, CD154, KIRDS2, CD2, CD27, LFA-1 (CD11a, CD18), GITR, CD40, BAFFR, HVEM (LIGHTR), SLAMF7, NKp80 (KLRF1), NKp44, NKp30, NKp46, CD160, CD19, IL2R beta, IL2R gamma, IL7Rx, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NG2C.

### Spacer Regions

In some embodiments, a CAR of the present disclosure comprises a spacer region or a hinge domain that links the extracellular antigen-binding domain to the transmembrane domain. A spacer or hinge domain may be any oligopeptide or polypeptide that functions to link the transmembrane domain to the extracellular domain and/or the intracellular signaling domain in the polypeptide chain. Spacer or hinge domains provide flexibility to the chimeric receptor, or domains thereof, or prevent steric hindrance of the chimeric receptor, or domains thereof. For example, the spacer region may be flexible enough to allow the antigen-binding domain to orient in different directions to facilitate antigen recognition. In some embodiments, the spacer region may be a hinge from a human protein. In some embodiments, a spacer domain or hinge domain may comprise up to 300 amino acids (e.g., 10 to 100 amino acids, or 5 to 20 amino acids). In some embodiments, one or more spacer domain(s) may be included in other regions of the chimeric receptor. For example, the hinge may be a human Ig (immunoglobulin) hinge, including without limitation an IgG4 hinge, an IgG2 hinge, a CD8a hinge, or an IgD hinge. In some embodiments, the spacer region comprises an IgG4 hinge, an IgG2 hinge, an IgD hinge, a CD28 hinge, a KIR2DS2 hinge, an LNGFR hinge, or a PDGFR-beta extracellular linker. In some embodiments, the spacer region is localized between the antigen-binding domain and the transmembrane domain.

TABLE 2
Exemplary spacer or finger domain sequences

| Name | Spacer/Hinge Domains |
|---|---|
| CD28 hinge | AAAIEVMYPPPYLDNEKSNGTIIHVKGKHL |
| | CPSPLFPGPSKP (SEQ ID NO: 139) |
| IgG4 minimal hinge | ESKYGPPCPSCP (SEQ ID NO: 140) |
| IgG4 minimal hinge, no | ESKYGPPAPSAP (SEQ ID NO: 141) |
| disulfides | |
| IgG4 S228P minimal hinge, | ESKYGPPCPPCP (SEQ ID NO: 142) |
| enhanced disulfide | |
| formation | |
| IgG1 minimal hinge | EPKSCDKTHTCP (SEQ ID NO: 143) |
| Extended CD8a hinge | AAAFVPVFLPAKPTTTPAPRPPTPAPTIASQ |
| | PLSLRPEACRPAAGGAVHTRGLDFACDIYI |
| | WAPLAGTCGVLLLSLVITLYCNHRN (SEQ |
| | ID NO: 144) |
| CD8a hinge | TTTPAPRPPTPAPTIALQPLSLRPEACRPAA |
| | GGAVHTRGLDFACD (SEQ ID NO: 145) |
| LNGFR hinge | ACPTGLYTHSGECCKACNLGEGVAQPCGA |
| | NQTVCEPCLDSVTFSDVVSATEPCKPCTEC |
| | VGLQSMSAPCVEADDAVCRCAYGYYQDE |
| | TTGRCEACRVCEAGSGLVFSCQDKQNTVC |
| | EECPDGTYSDEADAEC (SEQ ID NO: 146) |
| Truncated LNGFR hinge | ACPTGLYTHSGECCKACNLGEGVAQPCGA |
| (TNFR-Cys1) | NQTVC (SEQ ID NO: 147) |
| PDGFR-beta extracellular | AVGQDTQEVIVVPHSLPFKV (SEQ ID NO: |
| linker | 148) |

TABLE 3
Exemplary linker sequences.

| Linker | Amino Acid Sequence |
|---|---|
| (G₂S)₁ linker | GGS (SEQ ID NO: 149) |
| (G₂S)₂ linker | GGSGGS (SEQ ID NO: 150) |
| (G₂S)₃ linker | GGSGGSGGS (SEQ ID NO: 151) |
| (G₂S)₄ linker | GGSGGSGGSGGS (SEQ ID NO: 152) |
| (G₂S)₅ linker | GGSGGSGGSGGSGGS (SEQ ID NO: 153) |
| (G₃S)₁ linker | GGGS (SEQ ID NO: 154) |
| (G₃S)₂ linker | GGGSGGGS (SEQ ID NO: 155) |
| (G₃S)₃ linker | GGGSGGGSGGGS (SEQ ID NO: 156) |
| (G₃S)₄ linker | GGGSGGGSGGGSGGGS (SEQ ID NO: 157) |
| (G₃S)₅ linker | GGGSGGGSGGGSGGGSGGGS (SEQ ID NO: 158) |
| (G₄S)₁ linker | GGGGS (SEQ ID NO: 159) |
| (G₄S)₂ linker | GGGGSGGGGS (SEQ ID NO: 160) |
| (G₄S)₃ linker | GGGGSGGGGSGGGGS (SEQ ID NO: 161) |
| (G₄S)₄ linker | GGGGSGGGGSGGGGSGGGGS (SEQ ID NO: 162) |
| (G₄S)₅ linker | GGGGSGGGGSGGGGSGGGGSGGGGS (SEQ ID NO: 163) |
| (G₃S₂)₅ linker | GGSGS (SEQ ID NO: 164) |
| GS linker | GGCAGC (SEQ ID NO: 165) |
| Whitlow linker | GSTSGSGKPGSGEGSTKG (SEQ ID NO: 166) |
| Linker | EAAAKEAAAKEAAAKEAAAK (SEQ ID NO: 167) |

In some embodiments, one or more linker regions are present between the various domains of a CAR. For example, in some embodiments, a linker region can be positioned between a dimerization domain and a protease cleavage domain in the extracellular domain of the first polypeptide and/or the second polypeptide. In some embodiments, a linker region can be positioned between a dimerization domain and a VH or VL region of an antigen binding domain in the extracellular domain of the first polypeptide and/or the second polypeptide. In some embodiments, a linker region can be positioned between a VH region and a VL region of an antigen binding domain in the extracellular domain of the CAR. In some embodiments, a linker region can be positioned between an effector molecule and any of the above domains within the extracellular domain of the CAR. In some embodiments, a linker sequence can be repeated one or more times withing a linker region. In some embodiments, the first polypeptide and/or the second polypeptide comprises one or more linkers. In some embodiments, the one or more linkers comprise a GSG linker, a Whitlow linker, an eGK linker, or any derivative thereof.

### Intracellular Domains

In some embodiments, a CAR of the present disclosure comprises one or more cytoplasmic domains or regions. The cytoplasmic domain or region of the CAR may include an intracellular signaling domain.

Examples of intracellular signaling domains that may be used in CARs of the present disclosure include, without limitation, cytoplasmic sequences of the T cell receptor (TCR) and co-receptors that act in concert to modulate signal transduction following antigen receptor engagement, as well as any derivative or variant of these sequences and any recombinant sequence that has the same functional capability.

Without wishing to be bound by theory, it is believed that signals generated through the TCR alone are insufficient for full activation of the T cell and that a secondary and/or co-stimulatory signal is thus also typically utilized for full activation. Accordingly, T cell activation may be mediated by two distinct classes of cytoplasmic signaling sequences, those that initiate antigen-dependent primary activation through the TCR (primary intracellular signaling domains) and those that act in an antigen-independent manner to provide a secondary or co-stimulatory signal (secondary cytoplasmic domain, e.g., a co-stimulatory domain). In addition, T cell signaling and function (e.g., an activating signaling cascade) can be negatively regulated by inhibitory receptors present in a T cell through intracellular inhibitory co-signaling domains.

In some embodiments, the intracellular signaling domain of a CAR of the present disclosure includes an inhibitory intracellular signaling domains. In some embodiments, the inhibitory intracellular signaling domain includes one or more intracellular inhibitory co-signaling domains. In some embodiments, the one or more intracellular inhibitory co-signaling domains are linked to other domains (e.g., a transmembrane domain) through a peptide linker (e.g., see Table 6) or a spacer or hinge sequence (e.g., see Table 7). In some embodiments, when two or more intracellular inhibitory co-signaling domains are present, the two or more intracellular inhibitory co-signaling domains can be linked through a peptide linker or a spacer or hinge sequence (e.g., see Table 7). In some embodiments, the intracellular inhibitory co-signaling domain is an inhibitory domain. In some embodiments, the one or more intracellular inhibitory co-signaling domains of a chimeric protein comprises one or more ITIM-containing protein, or fragment(s) thereof. ITIMs are conserved amino acid sequences found in cytoplasmic tails of many inhibitory immune receptors. In some embodiments, the one or more ITIM-containing protein, or fragments thereof, is selected from PD-1, CTLA4, TIGIT, BTLA, and LAIR1. In some embodiments, the one or more intracellular inhibitory co-signaling domains comprise one or more non-ITIM scaffold proteins, or a fragment(s) thereof. In some embodiments, the one or more non-ITIM scaffold proteins, or fragments thereof, are selected from GRB-2, Dok-1, Dok-2, SLAP, LAG3, HAVR, GITR, and PD-L1. The inhibitory intracellular signaling domain can further include an enzymatic inhibitory domains. In some embodiments, the enzymatic inhibitory domain comprises an enzyme catalytic domain. In some embodiments, the enzyme catalytic domain is derived from an enzyme including, but not limited to, CSK, SHP-1, PTEN, CD45, CD148, PTP-MEG1, PTP-PEST, c-CBL, CBL-b, PTPN22, LAR, PTPH1, SHIP-1, or RasGAP. Examples of enzymatic regulation of signaling is described in more detail in Pavel Otáhal et al. (Biochim Biophys Acta. 2011 February; 1813(2):367-76), Kosugi A., et al. (Involvement of SHP-1 tyrosine phosphatase in TCR-mediated signaling pathways in lipid rafts, Immunity, 2001 June; 14(6): 669-80), and Stanford, et al. (Regulation of TCR signaling by tyrosine phosphatases: from immune homeostasis to autoimmunity, Immunology, 2012 September; 137(1): 1-19), each of which is incorporated herein by reference for all purposes.

In some embodiments, the intracellular signaling domain of a CAR of the present disclosure comprises a primary signaling domain that regulates primary activation of the TCR complex either in a stimulatory way or in an inhibitory way. Primary intracellular signaling domains that act in a stimulatory manner may contain signaling motifs which are known as immunoreceptor tyrosine-based activation motifs (ITAMs). Examples of suitable ITAM-containing primary intracellular signaling domains that may be used in the CARs of the present disclosure include, without limitation, those of CD3-zeta, FcR gamma, FcR beta, CD3 gamma, CD3 delta, CD3 epsilon, CD5, CD22, CD79a, CD79b, CD278 (also known as “ICOS”), FcΣRI, DAP10, DAP12, and CD66d.

In some embodiments, a CAR of the present disclosure comprises an intracellular signaling domain, e.g., a primary signaling domain of CD3-zeta polypeptide. A CD3-zeta polypeptide of the present disclosure may have an amino acid sequence that is at least 85%, at least 90%, at least 91%, at least 92%, at least 93%, at least 94%, at least 95%, at least 96%, at least 97%, at least 98%, at least 99%, or 100% homologous to the sequence of NCBI Reference No. NP_932170 or NP_001106864.2, or fragments thereof. In some embodiments, the CD3-zeta polypeptide may comprise one conservative amino acid substitution, up to two conservative amino acid substitutions, or up to three conservative amino acid substitutions. In some embodiments, the polypeptide can have an amino acid sequence that is a consecutive portion of NCBI Reference No. NP_932170 or NP_001106864.2 that is at least 20, at least 30, at least 40, at least 50, at least 60, at least 70, at least 80, at least 90, at least 100, at least 110, at least 120, at least 130, at least 140, at least 150, or at least 160, at least 170, or at least 180 amino acids in length.

In other embodiments, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In one embodiment, a primary signaling domain comprises one, two, three, four or more ITAM motifs.

In some embodiments, the one or more intracellular signaling domains is selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CDS intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.

A chimeric polypeptide of the present disclosure may be a first, second, or third generation CAR. First generation CARs comprise a single intracellular signaling domain, generally derived from a T cell receptor chain. First generation CARs generally have the intracellular signaling domain from the CD3-zeta (CD3ζ) chain, which is the primary transmitter of signals from endogenous TCRs. First generation CARs can provide de novo antigen recognition and cause activation of both CD4⁺ and CD8⁺ T cells through their CD3ζ chain signaling domain in a single fusion molecule, independent of HLA-mediated antigen presentation. Second generation CARs add a second intracellular signaling domain from one of various co-stimulatory molecules (e.g., CD28, 4-1BB, ICOS, OX40) to the cytoplasmic tail of the CAR to provide additional signals to the T cell. Second generation CARs provide both co-stimulation (e.g., CD28 or 4-1BB) and activation (CD3ζ). Third generation CARs have multiple intracellular co-stimulation signaling domains (e.g., CD28 and 4-1BB) and an intracellular activation signaling domain (CD3ζ).

In some embodiments, the intracellular signaling domain comprises a primary signaling domain (e.g., a primary signaling domain of CD3-zeta). In some embodiments, the intracellular signaling domain further comprises one or more functional signaling domains derived from at least one co-stimulatory molecule as defined below. In some embodiments, the co-stimulatory molecule is chosen from 4-1BB (i.e., CD137), CD27, ICOS, and/or CD28. In some embodiments, the chimeric polypeptides of the present disclosure comprise a chimeric fusion protein comprising an extracellular antigen-binding domain, a transmembrane domain and an intracellular signaling domain comprising a functional signaling domain derived from a stimulatory molecule. In some embodiments, the chimeric polypeptides of the present disclosure comprise a chimeric fusion protein comprising an extracellular antigen-binding domain, a transmembrane domain and an intracellular signaling domain comprising a functional signaling domain derived from a co-stimulatory molecule and a functional signaling domain derived from a stimulatory molecule. In some embodiments, the chimeric polypeptides of the present disclosure comprise a chimeric fusion protein comprising an extracellular antigen-binding domain, a transmembrane domain and an intracellular signaling domain comprising two functional signaling domains derived from one or more co-stimulatory molecule(s) and a functional signaling domain derived from a stimulatory molecule. In some embodiments, the chimeric polypeptides of the present disclosure comprise a chimeric fusion protein comprising an extracellular antigen-binding domain, a transmembrane domain and an intracellular signaling domain comprising at least two functional signaling domains derived from one or more co-stimulatory molecule(s) and a functional signaling domain derived from a stimulatory molecule.

In some embodiments, the first polypeptide and/or the second polypeptide of the chimeric protein systems of the present disclosure comprise a co-stimulatory domain including, but not limited to, a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, and a MyD88 intracellular signaling domain. In some aspects, the CAR comprises a CD3zeta-chain intracellular signaling domain and one or more additional intracellular signaling domains (e.g., co-stimulatory domains) selected from a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.

In some embodiments, a first polypeptide and/or a second polypeptide of the chimeric protein systems of the present disclosure comprise an intracellular cytoplasmic domain from LIR1, LIR2, LIR3, LIR5, LIR8, IRTA1, IRTA2, IRTA4, LAIR1, BTLA, KIR2DL1, KIR2DL2, KIR2DL3, KIR2DL4, KIR2DL5, KIR3DL1, KIR3DL2, NKIR, TLT1, PD-1, CTLA4, TIM-3, LAG3, TIGIT, FCRH3, FCGR2B, SIGLEC-2, SIGLEC-6, SIGLEC-7, SIGLEC-12, SIGLEC-10, KLRG1, NKG2A, CD72, PECAM-1, CEACAM-1, PCDHGC3, PCDHGC5, CDH11, PTPRO, PTPRZ1, SLAMF1, SLAMF5, CD45, IMPG2, DSCAM, LIFR, ERMAP, IL1RAP, IL1RAPL2, CDH5, MPZL1, MPZ, MPIG6B, VSIG4, SLAP1, SLAP2, dok-1, Dok-2, GRB-2, CD200R, SIRPa, HAVR, GITR, PDL-1, and/or CD94.

### Leader/Signal Sequences

In some embodiments, CARs of the present disclosure include a leader sequence (also referred to as a signal sequence) at the amino-terminus (N-term) of the CAR. In some embodiments, the CAR further comprises a leader sequence at the N-terminus of the extracellular antigen-binding domain, wherein the leader sequence is optionally cleaved from the antigen recognition domain (e.g., an scFv) during cellular processing and localization of the CAR to the cellular membrane.

Exemplary architectures of the chimeric polypeptides of the chimeric protein systems of the present disclosure are provided below in Table 6. Additionally, exemplary amino acid sequences of the various domains of the first polypeptide and/or the second polypeptide of the chimeric protein systems of the present disclosure are provided in Table 7 below.

### Chimeric Receptor Encoding Polynucleotide Constructs

In accordance with the above embodiments, the first and/or second polypeptides (e.g., CARs) of the chimeric protein systems of the present disclosure can be encoded by nucleic acid molecules. The present disclosure provides nucleic acid molecules encoding any of the polypeptides that comprise the dimerization domains described herein. In some embodiments, the present disclosure provides an engineered polynucleotide comprising an expression cassette that includes a promoter operably linked to an exogenous polynucleotide sequence encoding a CAR comprising a dimerization domain. As used herein, the term “promoter” generally refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

Promoters of an engineered nucleic acid of the present disclosure may be “inducible promoters,” which refer to promoters that are characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., cytokine) that contacts an inducible promoter in such a way as to be active in regulating transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

A promoter is “responsive to” or “modulated by” a local tumor state (e.g., inflammation or hypoxia) or signal if in the presence of that state or signal, transcription from the promoter is activated, deactivated, increased, or decreased. In some embodiments, the promoter comprises a response element. A “response element” is a short sequence of DNA within a promoter region that binds specific molecules (e.g., transcription factors) that modulate (regulate) gene expression from the promoter. Response elements that may be used in accordance with the present disclosure include, without limitation, a phloretin-adjustable control element (PEACE), a zinc-finger DNA-binding domain (DBD), an interferon-gamma-activated sequence (GAS) (Decker, T. et al. J Interferon Cytokine Res. 1997 March; 17(3):121-34, incorporated herein by reference), an interferon-stimulated response element (ISRE) (Han, K. J. et al. J Biol Chem. 2004 Apr. 9; 279(15):15652-61, incorporated herein by reference), a NF-kappaB response element (Wang, V. et al. Cell Reports. 2012; 2(4): 824-839, incorporated herein by reference), and a STAT3 response element (Zhang, D. et al. J of Biol Chem. 1996; 271:9503-9509, incorporated herein by reference). Other response elements are encompassed herein. Response elements can also contain tandem repeats (e.g., consecutive repeats of the same nucleotide sequence encoding the response element) to generally increase sensitivity of the response element to its cognate binding molecule. Tandem repeats can be labeled 2×, 3×, 4×, 5×, etc. to denote the number of repeats present.

Non-limiting examples of promoters include the cytomegalovirus (CMV) promoter, the elongation factor 1-alpha (EF1a) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter. In some embodiments, the promoter is a constitutive promoter.

In some embodiments, the promoter sequence is derived from a promoter selected from: minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule responsive promoters, and tandem repeats thereof. In some embodiments, the first promoter is a constitutive promoter, an inducible promoter, or a synthetic promoter. In some embodiments, the constitutive promoter is selected from: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

Non-limiting examples of responsive promoters (also referred to as “inducible promoters”) are listed in Table 4, which shows the design of the promoter and transcription factor, as well as the effect of the inducer molecule towards the transcription factor (TF) and transgene transcription (T) is shown (B, binding; D, dissociation; n.d., not determined) (A, activation; DA, deactivation; DR, derepression) (see Horner, M. & Weber, W. FEBS Letters 586 (2012) 20784-2096m, and references cited therein). Non-limiting examples of components of inducible promoters include those shown in Table 5.

TABLE 4
Exemplary Inducible Promoters

| | | | | Response to | |
|---|---|---|---|---|---|
| | Promoter and | Transcription | | inducer | |
| System | operator | factor (TF) | Inducer molecule | TF | T |
| Transcriptional activator-responsive promoters | | | | | |
| AIR | PAIR (OalcA- | AlcR | Acetaldehyde | n.d. | A |
| | PhCMVmin) | | | | |
| ART | PART (OARG- | ArgR-VP16 | l-Arginine | B | A |
| | PhCMVmin) | | | | |
| BIT | PBIT3 (OBirA3- | BIT (BirA- | Biotin | B | A |
| | PhCMVmin) | VP16) | | | |
| Cumate - | PCR5 (OCuO6- | cTA (CymR- | Cumate | D | DA |
| activator | PhCMVmin) | VP16) | | | |
| Cumate - | PCR5 (OCuO6- | rcTA (rCymR- | Cumate | B | A |
| reverse | PhCMVmin) | VP16) | | | |
| activator | | | | | |
| E-OFF | PETR (OETR- | ET (E-VP16) | Erythromycin | D | DA |
| | PhCMVmin) | | | | |
| NICE-OFF | PNIC (ONIC- | NT (HdnoR- | 6-Hydroxy-nicotine | D | DA |
| | PhCMVmin) | VP16) | | | |
| PEACE | PTtgR1 (OTtgR- | TtgA1 (TtgR- | Phloretin | D | DA |
| | PhCMVmin) | VP16) | | | |
| PIP-OFF | PPIR (OPIR- | PIT (PIP- | Pristinamycin I | D | DA |
| | Phsp70min) | VP16) | | | |
| QuoRex | PSCA (OscbR- | SCA (ScbR- | SCB1 | D | DA |
| | PhCMVmin)PSPA | VP16) | | | |
| | (OpapRI- | | | | |
| | PhCMVmin) | | | | |
| Redox | PROP (OROP- | REDOX | NADH | D | DA |
| | PhCMVmin) | (REX-VP16) | | | |
| TET-OFF | PhCMV*-1 (OtetO7- | tTA (TetR- | Tetracycline | D | DA |
| | PhCMVmin) | VP16) | | | |
| TET-ON | PhCMV*-1 (OtetO7- | rtTA (rTetR- | Doxycycline | B | A |
| | PhCMVmin) | VP16) | | | |
| TIGR | PCTA (OrhcO- | CTA (RhcA- | Heat | D | DA |
| | PhCMVmin) | VP16) | | | |
| TraR | O7x(tra box)- | p65-TraR | 3-Oxo-C8-HSL | B | A |
| | PhCMVmin | | | | |
| VAC-OFF | P1VanO2 (OVanO2- | VanA1 (VanR- | Vanillic acid | D | DA |
| | PhCMVmin) | VP16) | | | |
| Transcriptional repressor-responsive promoters | | | | | |
| Cumate - | PCuO (PCMV5- | CymR | Cumate | D | DR |
| repressor | OCuO) | | | | |
| E-ON | PETRON8 (PSV40- | E-KRAB | Erythromycin | D | DR |
| | OETR8) | | | | |
| NICE-ON | PNIC (PSV40- | NS (HdnoR- | 6-Hydroxy-nicotine | D | DR |
| | ONIC8) | KRAB) | | | |
| PIP-ON | PPIRON (PSV40- | PIT3 (PIP- | Pristinamycin I | D | DR |
| | OPIR3) | KRAB) | | | |
| Q-ON | PSCAON8 (PSV40- | SCS (ScbR- | SCB1 | D | DR |
| | OscbR8) | KRAB) | | | |
| TET- | OtetO-PHPRT | tTS-H4 (TetR- | Doxycycline | D | DR |
| ON<comma> | | HDAC4) | | | |
| repressor- | | | | | |
| based | | | | | |
| T-REX | PTetO (PhCMV- | TetR | Tetracycline | D | DR |
| | OtetO2) | | | | |
| UREX | PUREX8 (PSV40- | mUTS | Uric acid | D | DR |
| | OhucO8) | (KRAB-HucR) | | | |
| VAC-ON | PVanON8 (PhCMV- | VanA4 (VanR- | Vanillic acid | D | DR |
| | OVanO8) | KRAB) | | | |
| Hybrid promoters | | | | | |
| QuoRexPIP- | OscbR8-OPIR3- | SCAPIT3 | SCB1Pristinamycin I | DD | DADR |
| ON(NOT IF | PhCMVmin | | | | |
| gate) | | | | | |
| QuoRexE- | OscbR-OETR8- | SCAE-KRAB | SCB1Erythromycin | DD | DADR |
| ON(NOT IF | PhCMVmin | | | | |
| gate) | | | | | |
| TET-OFFE- | OtetO7-OETR8- | tTAE-KRAB | TetracyclineErythromycin | DD | DADR |
| ON(NOT IF | PhCMVmin | | | | |
| gate) | | | | | |
| TET-OFFPIP- | OtetO7-OPIR3- | tTAPIT3E- | TetracyclinePristinamycin | DDD | DADRDR |
| ONE-ON | OETR8-PhCMVmin | KRAB | IErythromycin | | |

TABLE 5
Exemplary Components of Inducible Promoters

| Name | DNA SEQUENCE | Source |
|---|---|---|
| minimal promoter; minP | AGAGGGTATATAATGGAAGC | EU581860.1 |
| | TCGACTTCCAG (SEQ ID NO: | (Promega) |
| | 168) | |
| NFKB response element protein | GGGAATTTCCGGGGACTTTC | EU581860.1 |
| promoter; 5x NFKB-RE | CGGGAATTTCCGGGGACTTT | (Promega) |
| | CCGGGAATTTCC (SEQ ID NO: | |
| | 169) | |
| CREB response element protein | CACCAGACAGTGACGTCAGC | DQ904461.1 |
| promoter; 4x CRE | TGCCAGATCCCATGGCCGTC | (Promega) |
| | ATACTGTGACGTCTTTCAGA | |
| | CACCCCATTGACGTCAATGG | |
| | GAGAA (SEQ ID NO: 170) | |
| NFAT response element protein | GGAGGAAAAACTGTTTCATA | DQ904462.1 |
| promoter; 3x NFAT binding | CAGAAGGCGTGGAGGAAAA | (Promega) |
| sites | ACTGTTTCATACAGAAGGCG | |
| | TGGAGGAAAAACTGTTTCAT | |
| | ACAGAAGGCGT (SEQ ID NO: | |
| | 171) | |
| SRF response element protein | AGGATGTCCATATTAGGACA | FJ773212.1 |
| promoter; 5x SRE | TCTAGGATGTCCATATTAGG | (Promega) |
| | ACATCTAGGATGTCCATATT | |
| | AGGACATCTAGGATGTCCAT | |
| | ATTAGGACATCTAGGATGTC | |
| | CATATTAGGACATCT (SEQ ID | |
| | NO: 172) | |
| SRF response element protein | AGTATGTCCATATTAGGACA | FJ773213.1 |
| promoter 2; 5x SRF-RE | TCTACCATGTCCATATTAGG | (Promega) |
| | ACATCTACTATGTCCATATT | |
| | AGGACATCTTGTATGTCCAT | |
| | ATTAGGACATCTAAAATGTC | |
| | CATATTAGGACATCT (SEQ ID | |
| | NO: 173) | |
| AP1 response element protein | TGAGTCAGTGACTCAGTGAG | JQ858516.1 |
| promoter; 6x AP1-RE | TCAGTGACTCAGTGAGTCAG | (Promega) |
| | TGACTCAG (SEQ ID NO: 174) | |
| TCF-LEF response element | AGATCAAAGGGTTTAAGATC | JX099537.1 |
| promoter; 8x TCF-LEF-RE | AAAGGGCTTAAGATCAAAG | (Promega) |
| | GGTATAAGATCAAAGGGCCT | |
| | AAGATCAAAGGGACTAAGA | |
| | TCAAAGGGTTTAAGATCAAA | |
| | GGGCTTAAGATCAAAGGGCC | |
| | TA (SEQ ID NO: 175) | |
| SBEx4 | GTCTAGACGTCTAGACGTCT | Addgene Cat No: 16495 |
| | AGACGTCTAGAC (SEQ ID | |
| | NO: 176) | |
| SMAD2/3-CAGACA x4 | CAGACACAGACACAGACAC | Jonk et al. (J Biol Chem. 1998 |
| | AGACA (SEQ ID NO: 177) | Aug 14;273(33): 21145-52. |
| STAT3 binding site | GGATCCGGTACTCGAGATCT | Addgene Sequencing Result |
| | GCGATCTAAGTAAGCTTGGC | #211335 |
| | ATTCCGGTACTGTTGGTAAA | |
| | GCCAC (SEQ ID NO: 178) | |
| 5x NFAT | GGGACTTTCCACTGGGGACT | |
| | TTCCACTGGGGACTTTCCAC | |
| | TGGGGACTTTCCACTGGGGA | |
| | CTTTCC (SEQ ID NO: 179) | |
| min AdeP | AGACGCTAGCGGGGGGCTAT | |
| | AAAAGGGGGTGGGGGCGTT | |
| | CGTCCTCACTCT (SEQ ID NO: | |
| | 180) | |
| 5x NFAT_minAdeP | GGGACTTTCCACTGGGGACT | |
| | TTCCACTGGGGACTTTCCAC | |
| | TGGGGACTTTCCACTGGGGA | |
| | CTTTCCACTCCTGCAGGagctG | |
| | GCGCGCCAGACGCTAGCGGG | |
| | GGGCTATAAAAGGGGGTGG | |
| | GGGCGTTCGTCCTCACTCT | |
| | (SEQ ID NO: 181) | |
| YB-TATA | TCTAGAGGGTATATAATGGG | |
| | GGCCA (SEQ ID NO: 182) | |

### Multicistronic and Multiple Promoter Systems

In some embodiments, engineered nucleic acids of the present disclosure can be multicistronic or polycistronic (e.g., polynucleotides encoding more than one CAR comprising a dimerization domain produced from a single mRNA transcript). Engineered nucleic acids can be multicistronic or polycistronic through the use of various linkers (e.g., a polynucleotide sequence encoding a CAR comprising a dimerization domain can be linked to a nucleotide sequence encoding a second exogenous polynucleotide), such as in a first gene:linker:second gene 5′ to 3′ orientation. A linker polynucleotide sequence can encode a 2A ribosome skipping element, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage. A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker is a Furin-Gly-Ser-Gly-2A fusion polypeptide. In some embodiments, a linker is a Furin-Gly-Ser-Gly-T2A fusion polypeptide. In general, a multicistronic or polycistronic system can use any number or combination of linkers, to express any number of genes or portions thereof (e.g., an engineered nucleic acid can encode a first, a second, and a third chimeric polypeptide, each separated by linkers such that separate chimeric polypeptides are produced). “Linkers,” as used herein can refer to polypeptides that link a first polypeptide sequence and a second polypeptide sequence or the multicistronic linkers described above.

In some embodiments, an engineered nucleic acid of the present disclosure comprises a post-transcriptional regulatory element (PRE). PREs can enhance gene expression via enabling tertiary RNA structure stability and 3′ end formation. Non-limiting examples of PREs include the Hepatitis B virus PRE (HPRE) and the Woodchuck Hepatitis Virus PRE (WPRE). In some embodiments, the post-transcriptional regulatory element is a Woodchuck Hepatitis Virus Posttranscriptional Regulatory Element (WPRE). In some embodiments, the WPRE comprises the alpha, beta, and gamma components of the WPRE element. In some embodiments, the WPRE comprises the alpha component of the WPRE element.

### 3. Engineered Immune Effector Cells

Also provided herein are cells, and methods of producing cells, which comprise one or more engineered nucleic acids of the present disclosure. These cells are referred to herein as “engineered cells.” These cells, which typically contain one or more engineered nucleic acids, do not occur in nature. In some embodiments, the cells are isolated cells that recombinantly express the one or more engineered nucleic acids. In some embodiments, the engineered one or more nucleic acids are expressed from one or more vectors or a selected locus from the genome of the cell. In some embodiments, the cells are engineered to include a nucleic acid comprising a promoter operable linked to a nucleic acid sequence encoding a chimeric polypeptide (e.g., CAR) comprising any of the dimerization domains described herein.

An engineered cell of the present disclosure can comprise an engineered nucleic acid integrated into the cell's genome. An engineered cell can comprise an engineered nucleic acid capable of expression without integrating into the cell's genome, for example, engineered with a transient expression system such as a plasmid or mRNA. In some embodiments, the engineered nucleic acid is selected from: a DNA, a cDNA, an RNA, an mRNA, and a naked plasmid. Also provided herein is an expression vector comprising the engineered nucleic acid.

An engineered cell or isolated cell of the present disclosure can be a human cell. An engineered cell or isolated cell can be a human primary cell. An engineered primary cell can be a tumor infiltrating primary cell. An engineered primary cell can be a primary T cell. An engineered primary cell can be a hematopoietic stem cell (HSC). An engineered primary cell can be a natural killer (NK) cell. An engineered primary cell can be any somatic cell. An engineered primary cell can be an MSC. In some embodiments, the engineered cell is derived from the subject. In some embodiments, the engineered cell is allogeneic with reference to the subject.

An engineered cell of the present disclosure can be isolated from a subject, such as a subject known or suspected to have cancer. Cell isolation methods are known to those skilled in the art and include, but are not limited to, sorting techniques based on cell-surface marker expression, such as FACS sorting, positive isolation techniques, and negative isolation, magnetic isolation, and combinations thereof. An engineered cell can be allogenic with reference to the subject being administered a treatment. Allogenic modified cells can be HLA-matched to the subject being administered a treatment. An engineered cell can be a cultured cell, such as an ex vivo cultured cell. An engineered cell can be an ex vivo cultured cell, such as a primary cell isolated from a subject. Cultured cell can be cultured with one or more cytokines.

In some embodiments, an engineered or isolated cell of the present disclosure is selected from: a T cell, a CD8+ T cell, a CD4+ T cell, a gamma-delta T cell, a cytotoxic T lymphocyte (CTL), a regulatory T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell. In some embodiments, the engineered cell is a Natural Killer (NK) cell. In some embodiments, the engineered cell is a CD3⁻ and/or CD56⁺. In some embodiments, an engineered cell is autologous. In some embodiments, an engineered cell is allogeneic.

In some embodiments, an engineered cell of the present disclosure is a tumor cell selected from: an adenocarcinoma cell, a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, a mesothelioma cell, an ovarian tumor cell, a pancreatic tumor cell, a gastric tumor cell, a testicular yolk sac tumor cell, a prostate tumor cell, a skin tumor cell, a thyroid tumor cell, and a uterine tumor cell.

Also provided herein are methods that include culturing the engineered cells of the present disclosure. Methods of culturing the engineered cells described herein are known. One skilled in the art will recognize that culturing conditions will depend on the particular engineered cell of interest. One skilled in the art will recognize that culturing conditions will depend on the specific downstream use of the engineered cell, for example, specific culturing conditions for subsequent administration of the engineered cell to a subject.

Embodiments of the present disclosure also include compositions and methods for engineering cells to produce the chimeric polypeptides (e.g., CARs) comprising dimerization domains. In general, cells are engineered to produce chimeric polypeptides (e.g., CARs) comprising dimerization domains through introduction (i.e., delivery) of one or more polynucleotides of the present disclosure comprising a promoter and an exogenous polynucleotide sequence encoding a chimeric polypeptide comprising a dimerization domain into the cell's cytosol and/or nucleus. For example, the polynucleotide expression cassettes encoding the chimeric polypeptides comprising dimerization domains can be any of the engineered nucleic acids described herein. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

### 4. Delivery Systems

Viral-Mediated Delivery. Viral vector-based delivery platforms can be used to engineer cells. In general, a viral vector-based delivery platform engineers a cell through introducing (i.e., delivering) into a host cell. For example, a viral vector-based delivery platform can engineer a cell through introducing any of the engineered nucleic acids described herein. A viral vector-based delivery platform can be a nucleic acid, and as such, an engineered nucleic acid can also encompass an engineered virally-derived nucleic acid. Such engineered virally-derived nucleic acids can also be referred to as recombinant viruses or engineered viruses.

A viral vector-based delivery platform can encode more than one engineered nucleic acid, gene, or transgene within the same nucleic acid. For example, an engineered virally-derived nucleic acid (e.g., a recombinant virus or an engineered virus) can encode one or more transgenes, including, but not limited to, any of the chimeric polypeptides described herein that include one or more dimerization domains. A viral vector-based delivery platform can encode one or more genes in addition to the one or more chimeric polypeptides, such as viral genes needed for viral infectivity and/or viral production (e.g., capsid proteins, envelope proteins, viral polymerases, viral transcriptases, etc.), referred to as cis-acting elements or genes. In general, any of the viral vector-based systems can be used for the in vitro production of a chimeric polypeptide, or used in vivo and ex vivo gene therapy procedures, e.g., for in vivo delivery of the engineered nucleic acids encoding the chimeric polypeptides. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, an adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (see, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The viral vector-based delivery platforms can be a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., an engineered nucleic acid) encoding a chimeric polypeptide comprising a dimerization domain. In some embodiments, the virus is selected from a lentivirus, a retrovirus, an oncolytic virus, an adenovirus, an adeno-associated virus (AAV), and a virus-like particle (VLP).

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids into the target cell to provide permanent transgene expression. Retroviral-based delivery systems include, but are not limited to, those based upon murine leukemia, virus (MuLV), gibbon ape leukemia virus (GaLV), Simian Immuno deficiency vims (SIV), human immuno deficiency vims (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of chimeric polypeptides comprising a dimerization domain, or used in vivo and ex vivo gene therapy procedures, e.g., for in vivo delivery of the engineered nucleic acids encoding one or more chimeric polypeptides comprising a dimerization domain (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78(12):6381-6388 (June 2004); Gao, et al, Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat &amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

Lipid Structure Delivery Systems. Engineered nucleic acids of the present disclosure (e.g., any of the engineered nucleic acids described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain aspects, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to embodiments of the present disclosure can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes. Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety. Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Felgner et al., Proc. Natl. Acad. Sci. USA 84:7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane, and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of an engineered nucleic acid and/or viral delivery system can be used to avoid degradation, while also avoiding potential off-target affects. In certain examples, an engineered nucleic acid and/or viral delivery system is fully encapsulated within the delivery vehicle, such as within the aqueous interior of an LNP. Encapsulation of an engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with an engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., an engineered nucleic acid and/or viral delivery system) can be further treated or engineered to prepare them for administration.

Nanoparticle Delivery. Nanomaterials can be used to deliver engineered nucleic acids (e.g., any of the engineered nucleic acids described herein). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery—A Review. Nanomaterials 2017, 7(5), 94), herein incorporated by reference for all purposes.

Genomic Editing Systems. A genomic editing system can be used to engineer a host genome to encode an engineered nucleic acid, such as an engineered nucleic acid of the present disclosure. In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate an engineered nucleic acid, such as an engineered nucleic acid of the present disclosure, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tc1/mariner transposon superfamily, such as a Sleeping Beauty transposon system, described in more detail in Hudecek et al. (Crit Rev Biochem Mol Biol. 2017 August; 52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode an engineered nucleic acid, such as an engineered nucleic acid of the present disclosure. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein.

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid, such as an engineered nucleic acid encoding one or more chimeric polypeptides described herein comprising a dimerization domain. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and an RNA(s) that directs cleavage to a particular target sequence. An exemplary CRISPR-mediated gene editing system is the CRISPR/Cas9 systems comprised of a Cas9 nuclease and an RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The engineered cells described herein can be engineered using non-viral methods, e.g., the nuclease and/or CRISPR mediated gene editing systems described herein can be delivered to a cell using non-viral methods. The engineered cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA-binding domain of TALE (transcription activator-like effectors) and the catalytic domain of restriction endonuclease Fokl. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

Other Engineering Delivery Systems. Various additional means to introduce engineered nucleic acids (e.g., any of the engineered nucleic acids described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Compositions and methods for delivering engineered mRNAs in vivo, such as naked plasmids or mRNA, are described in detail in Kowalski et al. (Mol Ther. 2019 Apr. 10; 27(4): 710-728) and Kaczmarek et al. (Genome Med. 2017; 9:60.), each herein incorporated by reference for all purposes.

Other means for introducing engineered nucleic acids (e.g., any of the engineered nucleic acids described herein) into a cell or other target recipient entity include, but are not limited to, sonication, gene gun, hydrodynamic injection, and cell membrane deformation by physical means.

### 5. Compositions and Methods of Use

Embodiments of the present disclosure include a pharmaceutical composition comprising any of the engineered cells (e.g., NK cells) described herein comprising the chimeric polypeptides (e.g., CARs) having dimerization domains, and a pharmaceutically acceptable carrier, pharmaceutically acceptable excipient, or a combination thereof. In accordance with these embodiments, the present disclosure includes a method of treating a subject in need thereof (e.g., a subject diagnosed with cancer or suspected of having cancer). In some embodiments, the method includes administering a therapeutically effective dose of any of the engineered cells of the present disclosure. In some embodiments, the subject has a proliferative disease, an immunological disease, a metabolic disease, a genetic disease, an ophthalmological disease, a cardiovascular disease, or a neurological disease.

Embodiments of the present disclosure also include a method of treating a subject by administering a pharmaceutical composition comprising any of the engineered cells (e.g., NK cells) described herein comprising the chimeric polypeptides (e.g., CARs) having dimerization domains. In some embodiments, the subject has a proliferative disease, an immunological disease, a metabolic disease, a genetic disease, an ophthalmological disease, a cardiovascular disease, or a neurological disease.

Embodiments of the present disclosure also include a kit for treating and/or preventing a tumor comprising any of the engineered cells (e.g., NK cells) described herein comprising the chimeric polypeptides (e.g., CARs) having dimerization domains, and a container. In some embodiments, the kit further comprises written instructions for using the engineered cell for treating and/or preventing a tumor in a subject. In some embodiments, the kit comprises a pharmaceutical composition comprising any of the engineered cells described herein, and a pharmaceutically acceptable carrier, pharmaceutically acceptable excipient, or a combination thereof. In some embodiments, the kit further comprises written instructions for using the pharmaceutical composition for treating and/or preventing a tumor in a subject.

In some embodiments, provided herein are methods of stimulating a cell-mediated immune response to a tumor cell in a subject. In some embodiments, the method includes administering to a subject having a tumor a therapeutically effective dose of any of the engineered cells, isolated cells, or compositions disclosed herein. In some embodiments, provided herein are methods of providing an anti-tumor immunity in a subject. In some embodiments, the method includes administering to a subject in need thereof a therapeutically effective dose of any of the engineered cells, isolated cells, or compositions disclosed herein. In some embodiments, provided herein are methods of treating a subject having cancer. In some embodiments, the method includes administering a therapeutically effective dose of any of the engineered cells, isolated cells, or compositions disclosed herein. In some embodiments, provided herein are methods of reducing tumor volume in a subject. In some embodiments, the method includes administering to a subject having a tumor a composition comprising any of the engineered cells, isolated cells, or compositions disclosed herein. In some embodiments, administration includes systemic administration. In some embodiments, administration includes intratumoral administration. In some embodiments, the isolated cell is derived from the subject. In some embodiments, the isolated cell is allogeneic with reference to the subject.

In some embodiments, the tumor is selected from an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.

Some methods comprise selecting a subject (or patient population) having a tumor (or cancer) and treating that subject with engineered cells or delivery vehicles that modulate tumor-mediated immunosuppressive mechanisms.

The methods provided herein also include delivering a preparation of engineered cells or delivery vehicles. A preparation, in some embodiments, is a substantially pure preparation, containing, for example, less than 5% (e.g., less than 4%, 3%, 2%, or 1%) of cells other than engineered cells. A preparation may comprise 1×10⁵ cells/kg to 1×10⁷ cells/kg cells.

The methods provided herein also include administering a drug or pharmaceutical composition in combination with a therapeutically effective dose of any of the engineered cells, isolated cells, or compositions disclosed herein. The drug or pharmaceutical can be administered prior to, concurrently with, simultaneously with, and/or subsequent to administration of any of the engineered cells, isolated cells, or compositions disclosed herein. The drug or pharmaceutical can be administered serially. The drug or pharmaceutical can be administered concurrently or simultaneously with administration of any of the engineered cells, isolated cells, or compositions disclosed herein. The drug or pharmaceutical can be administered at separate intervals than (e.g., prior to or subsequent to) administration of any of the engineered cells, isolated cells, or compositions disclosed herein. The drug or pharmaceutical can be administered both concurrently/simultaneously as well as at separate intervals than any of the engineered cells, isolated cells, or compositions disclosed herein. The drug or pharmaceutical composition and the engineered cells, isolated cells, or compositions can be administered via different routes, e.g., the drug or pharmaceutical composition can be administered orally, and the engineered cells, isolated cells, or compositions can be administered intraperitoneally, intravenously, subcutaneously, or any other route appropriate for administration, as will be appreciated by one skilled in the art.

The methods provided herein also include delivering a composition in vivo capable of producing the engineered cells described herein, e.g., capable of delivering any of the engineered nucleic acids described herein to a cell in vivo. Such compositions include any of the viral-mediated delivery platforms, any of the lipid structure delivery systems, any of the nanoparticle delivery systems, any of the genomic editing systems, or any of the other engineering delivery systems described herein capable of engineering a cell in vivo.

The engineered nucleic acid or engineered cell can be formulated in pharmaceutical compositions. These compositions can comprise, in addition to one or more of the engineered nucleic acids or engineered cells, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

Whether it is a polypeptide, nucleic acid, small molecule or other pharmaceutically useful compound according to the present disclosure that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

Aspects of the present disclosure include a kit for treating and/or preventing a tumor. In some embodiments, the kit comprises any of the immunoresponsive cells described herein. In some embodiments, the kit further comprises written instructions for using the immunoresponsive cell for treating and/or preventing a tumor in a subject. Aspects of the present disclosure include a kit for treating and/or preventing a tumor. In some embodiments, the kit comprises any of the pharmaceutical compositions described herein. In some embodiments, the kit further comprises written instructions for using the pharmaceutical composition for treating and/or preventing a tumor in a subject.

Certain aspects of the present disclosure relate to kits for the treatment and/or prevention of a cancer (e.g., solid tumors). In some embodiments, the kit includes a therapeutic or prophylactic composition comprising an effective amount of immune effector cells comprising one or more chimeric polypeptides comprising the dimerization domains of the present disclosure, isolated nucleic acids of the present disclosure, vectors of the present disclosure, and/or cells of the present disclosure (e.g., immune effector cells). In some embodiments, the kit comprises a sterile container. In some embodiments, such containers can be boxes, ampules, bottles, vials, tubes, bags, pouches, blister-packs, or other suitable container forms known in the art. The container may be made of plastic, glass, laminated paper, metal foil, or other materials suitable for holding medicaments.

In some embodiments, therapeutic or prophylactic composition is provided together with instructions for administering the therapeutic or prophylactic composition to a subject having or at risk of developing cancer (e.g., a solid tumor). In some embodiments, the instructions may include information about the use of the composition for the treatment and/or prevention of the disorder. In some embodiments, the instructions include, without limitation, a description of the therapeutic or prophylactic composition, a dosage schedule, an administration schedule for treatment or prevention of the disorder or a symptom thereof, precautions, warnings, indications, counter-indications, over-dosage information, adverse reactions, animal pharmacology, clinical studies, and/or references. In some embodiments, the instructions can be printed directly on the container (when present), or as a label applied to the container, or as a separate sheet, pamphlet, card, or folder supplied in or with the container.

### Enumerated Embodiments

Embodiment 1: A chimeric protein system comprising: a first polypeptide comprising a first antigen binding domain, a first transmembrane domain, and a first dimerization domain; and a second polypeptide comprising a second antigen binding domain, a second transmembrane domain, and a second dimerization domain; wherein the first dimerization domain is capable of binding to the second dimerization domain.
Embodiment 2: The chimeric protein system of embodiment 1, wherein the first dimerization domain or the second dimerization domain is selected from the group consisting of CD94, NKG2A, and NKG2C.
Embodiment 3: The chimeric protein system of embodiment 1 or 2, wherein the first dimerization domain comprises a dimerization domain from CD94, and the second dimerization domain comprises a dimerization domain from NKG2A or a dimerization domain from NKG2C.
Embodiment 4: The chimeric protein system of embodiment 1 or 2, wherein the first dimerization domain comprises a dimerization domain from NKG2A or a dimerization domain from NKG2C, and the second dimerization domain comprises a dimerization domain from CD94.
Embodiment 5: The chimeric protein system of any one of embodiments 1 to 4, wherein the first dimerization domain comprises a sequence selected from the group consisting of SEQ ID NOs: 70, 72, 74, 76, 78, 80, 82, 84, 86, 88, and 90.
Embodiment 6: The chimeric protein system of any one of embodiments 1 to 4, wherein the second dimerization domain comprises a sequence selected from the group consisting of SEQ ID NO: 70, 72, 74, 76, 78, 80, 82, 84, 86, 88, and 90.
Embodiment 7: The chimeric protein system of any one of embodiments 1 to 6, wherein the first polypeptide is a chimeric antigen receptor.
Embodiment 8: The chimeric protein system of embodiment 7, wherein the first polypeptide is an activating chimeric antigen receptor.
Embodiment 9: The chimeric protein system of embodiment 7, wherein the first polypeptide is an inhibitory chimeric antigen receptor.
Embodiment 10: The chimeric protein system of any one of embodiments 1 to 9, wherein the second polypeptide is a chimeric antigen receptor.
Embodiment 11: The chimeric protein system of embodiment 10, wherein the second polypeptide is an activating chimeric antigen receptor.
Embodiment 12: The chimeric protein system of embodiment 11, wherein the second polypeptide is an inhibitory chimeric antigen receptor.
Embodiment 13: The chimeric protein system of any one of embodiments 1 to 12, wherein the antigen binding domain comprises a F(ab) fragment, a F(ab′) fragment, a single chain variable fragment (scFv), a single domain antibody, a diabody, a VHH fragment, or a synthetic epitope.
Embodiment 14: The chimeric protein system of embodiment 13, wherein the antigen binding domain binds an antigen expressed on a cancer cell.
Embodiment 15: The chimeric protein system of embodiment 14, wherein the cancer comprises glioblastoma, neuroblastoma, breast cancer, colorectal cancer, prostate cancer, bladder cancer, liver cancer, lung cancer, pancreatic cancer, ovarian cancer, gastric cancer, endometrial cancer, cervical cancer, leukemia, lymphoma, or myeloma.
Embodiment 16: The chimeric protein system of any one of embodiments 1 to 15, wherein the antigen binding domain is specific for carcinoembryonic antigen (CEA), Mesothelin, Ax1, GPC3, FLT3, CD33, TROP2, MUC1, MUC16, IL13Ra, ErbB2 (HER2/neu), epithelial cell adhesion molecule (EpCAM), epidermal growth factor receptor (EGFR), EGFR variant III (EGFRvIII), CD19, CD20, CD30, CD40, disialoganglioside GD2, ductal-epithelial mucine, gp36, TAG-72, glycosphingolipids, glioma-associated antigen, alphafetoprotein (AFP), lectin-reactive AFP, thyroglobulin, RAGE 1, MN-CA IX, human telomerase reverse transcriptase, RU1, RU2 (AS), intestinal carboxyl esterase, mut hsp70-2, M-CSF, prostate specific antigen (PSA), PAP, NY-ESO-1, LAGA-1a, p53, prostein, PSMA, prostate-carcinoma tumor antigen-1 (PCTA-1), MAGE, ELF2M, neutrophil elastase, ephrin B2, CD22, insulin growth factor (IGF)-1, IGF-11, IGF-1 receptor, NKG2D, BCMA (CD269, TNFRSF 17), Claudin18.2, B7-H3, or Ror1.
Embodiment 17: The chimeric protein system of any one of embodiments 1 to 16, wherein the transmembrane domain is selected from the group consisting of: a LAX transmembrane domain, a CD25 transmembrane domain, a CD7 transmembrane domain, a LAT transmembrane domain, a transmembrane domain from a LAT mutant, a BTLA transmembrane domain, a CDS transmembrane domain, a CD28 transmembrane domain, a CD3zeta transmembrane domain, a CD4 transmembrane domain, a 4-IBB transmembrane domain, an OX40 transmembrane domain, an ICOS transmembrane domain, a 2B4 transmembrane domain, a PD-1 transmembrane domain, a CTLA4 transmembrane domain, a BTLA transmembrane domain, a TIM3 transmembrane domain, a LIR1 transmembrane domain, an NKG2A transmembrane domain, a TIGIT transmembrane domain, and a LAG3 transmembrane domain, a LAIR1 transmembrane domain, a GRB-2 transmembrane domain, a Dok-1 transmembrane domain, a Dok-2 transmembrane domain, a SLAP1 transmembrane domain, a SLAP2 transmembrane domain, a CD200R transmembrane domain, an SIRPa transmembrane domain, an HAVR transmembrane domain, a GITR transmembrane domain, a PD-L1 transmembrane domain, a KIR2DL1 transmembrane domain, a KIR2DL2 transmembrane domain, a KIR2DL3 transmembrane domain, a KIR3DL1 transmembrane domain, a KIR3DL2 transmembrane domain, a CD94 transmembrane domain, a KLRG-1 transmembrane domain, a PAG transmembrane domain, a CD45 transmembrane domain, and a CEACAM1 transmembrane domain.
Embodiment 18: The chimeric protein system of any one of embodiments 1 to 17, wherein the first polypeptide and/or the second polypeptide comprises one or more intracellular signaling domains.
Embodiment 19: The chimeric protein system of embodiment 18, wherein the one or more intracellular signaling domains are selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CDS intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.
Embodiment 20: The chimeric protein system of embodiment 18 or 19, wherein the one or more intracellular signaling domains comprises a co-stimulatory domain selected from the group consisting of: a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CDS intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.
Embodiment 21: The chimeric protein system of any one of embodiments 1 to 20, wherein the first polypeptide and/or the second polypeptide comprises a hinge domain located between the antigen binding domain and the transmembrane domain.
Embodiment 22: The chimeric protein system of any one of embodiments 1 to 21, wherein the first polypeptide and/or the second polypeptide comprises one or more linkers.
Embodiment 23: The chimeric protein system of embodiment 22, wherein the one or more linkers comprise a GSG linker, a Whitlow linker, an eGK linker, or any derivative thereof.
Embodiment 24: An engineered polynucleotide encoding the first polynucleotide and/or the second polynucleotide of any one of embodiments 1 to 23.
Embodiment 25: An expression vector comprising the engineered polynucleotide of embodiment 24.
Embodiment 26: An engineered cell comprising the engineered polynucleotide of embodiment 25, the vector of embodiment 25, or the first and/or second polypeptides of any one of embodiments 1 to 24.
Embodiment 27: The engineered cell of embodiment 26, wherein the cell is selected from the group consisting of: a T cell, a CDS+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.
Embodiment 28: The engineered cell of embodiment 26 or 27, wherein the cell is engineered to express an effector molecule.
Embodiment 29; The engineered cell of embodiment 28, wherein the cell is a Natural Killer (NK) cell.
Embodiment 30; The engineered cell of any one of embodiments 26 to 29, wherein the cell is autologous.
Embodiment 31: The engineered cell of any one of embodiments 26 to 30, wherein the cell is allogeneic.
Embodiment 32: A pharmaceutical composition comprising the engineered cell of any one of embodiments 26 to 31, and a pharmaceutically acceptable carrier and/or a pharmaceutically acceptable excipient.
Embodiment 33: A method of treating a subject having cancer, the method comprising administering to the subject a therapeutically effective dose of the composition of embodiment 32, or a cell of any one of embodiments 26 to 31 to the subject.
Embodiment 34: The method of embodiment 35, wherein the cancer comprises glioblastoma, neuroblastoma, breast cancer, colorectal cancer, prostate cancer, bladder cancer, liver cancer, lung cancer, pancreatic cancer, ovarian cancer, gastric cancer, endometrial cancer, cervical cancer, leukemia, lymphoma, or myeloma.
Embodiment 35: A method of enhancing immune cell-mediated killing of a cancer cell in a subject in need thereof, the method comprising administering a therapeutically effective dose of the composition of embodiment 32, or a cell of any one of embodiments 26 to 31, to the subject.
Embodiment 36: The method of embodiment 35, wherein the cancer comprises a solid tumor.
Embodiment 37: The method of embodiment 35 or embodiment 36, wherein the cancer comprises glioblastoma, neuroblastoma, breast cancer, colorectal cancer, prostate cancer, bladder cancer, liver cancer, lung cancer, pancreatic cancer, ovarian cancer, gastric cancer, endometrial cancer, cervical cancer, leukemia, lymphoma, or myeloma.
Embodiment 38: A method of reducing off-target killing of a healthy cell in a subject, the method comprising administering a therapeutically effective dose of the composition of embodiment 32, or a cell of any one of embodiments 26 to 31, to the subject, wherein the subject has been diagnosed with cancer.
Embodiment 39: The method of embodiment 38, wherein the cancer comprises a solid tumor.
Embodiment 40: The method of embodiment 38 or embodiment 39, wherein the cancer comprises glioblastoma, neuroblastoma, breast cancer, colorectal cancer, prostate cancer, bladder cancer, liver cancer, lung cancer, pancreatic cancer, ovarian cancer, gastric cancer, endometrial cancer, cervical cancer, leukemia, lymphoma, or myeloma.

### EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

In accordance with the various embodiments described herein, experiments were conducted to evaluate the efficacy of engineering chimeric antigen receptors (CARs) having complementary or opposite functions (e.g., activating vs. inhibitory) with dimerization domains to facilitate their co-localization (e.g., immune synapse) in effector cells. As described further herein, it is important for certain CARs (e.g., activating CARs and inhibitory CARs) to be expressed in close proximity to each other in order to execute a desired function (e.g., inhibition of an activating signal). The data provided herein demonstrate that the use of dimerization domains within the extracellular domains of CARs facilitates their co-localization, which enhances their synergy and/or competing effects.

In particular, experiments were conducted to evaluate the efficacy of generating an activating CAR (aCAR) and an inhibitory CAR (iCAR) with CD94/NKG2C or CD94/NKG2A dimerization domains, and their ability to modulate effector cell function. Exemplary aCAR/iCAR architectures are shown below in Table 6.

TABLE 6
Exemplary aCAR/iCAR architecture.

| | | | | |
|---|---|---|---|---|
| | Antigen | | Trans- | Intracellular |
| Signal | Binding | Hinge | membrane | Signaling |
| Sequence | Domain | Region | Domain | Domain |

aCARs:

| | | | | | |
|---|---|---|---|---|---|
| SB07524 | CD8 | CEA | CD94 | CD28z | CD28 |
| SB07526 | CD8 | CEA | NKG2A | CD28z | CD28 |
| SB07527 | CD8 | CEA | NKG2C | CD28z | CD28 |

iCARs:

| | | | | | |
|---|---|---|---|---|---|
| SB07530 | CD8 | VSIG2 | CD94 | LIR1 | LIR1 |
| SB07532 | CD8 | VSIG2 | NKG2A | LIR1 | LIR1 |
| SB07533 | CD8 | VSIG2 | NKG2C | LIR1 | LIR1 |

In accordance with the above, donor NK cells (donor #13 and donor #15) were transduced with various aCAR/iCAR combinations (Table 6) and their efficacy was assessed using Ls174t target cells that were transduced with or without VSIG2 (V-set and Immunoglobulin Domain Containing 2 is a membrane protein that is uniquely expressed in CEA-positive healthy cells but not in tumor cells). NK cell efficacy was assessed using Incucyte and flow cytometry based killing assays. Briefly, target cells were engineered to express a fluorescent protein (e.g., mKate or mCherry) with or without VSIG2. They were seeded at about 50,000 target cells per well. Target cell area was measured and quantified using Incucyte live microscopy. NK donor cells were engineered to express a particular aCAR/iCAR combination capable of dimerization via CD94/NKG2C or CD94/NKG2A dimerization domains (expressed via separate plasmids). The NK cells were added to the wells containing the target cells at a particular effector-to-target cell ratio (e.g., 1:2). Images of each well, including fluorescence images, were acquired every four hours using Incucyte live microscopy. Every 2-3 days, the engineered NK cells were collected and added to freshly seeded target cells for another round of imaging and quantification. Fluorescence area was used to determine target cell growth, which was normalized to t=0. The killing of target cells not expressing VSIG2 by NK cells comprising the aCAR/iCAR combination was assessed (no iCAR inhibition of the aCAR), along with the killing of target cells expressing VSIG2 by NK cells comprising the aCAR/iCAR combination (aCAR inhibition by the iCAR).

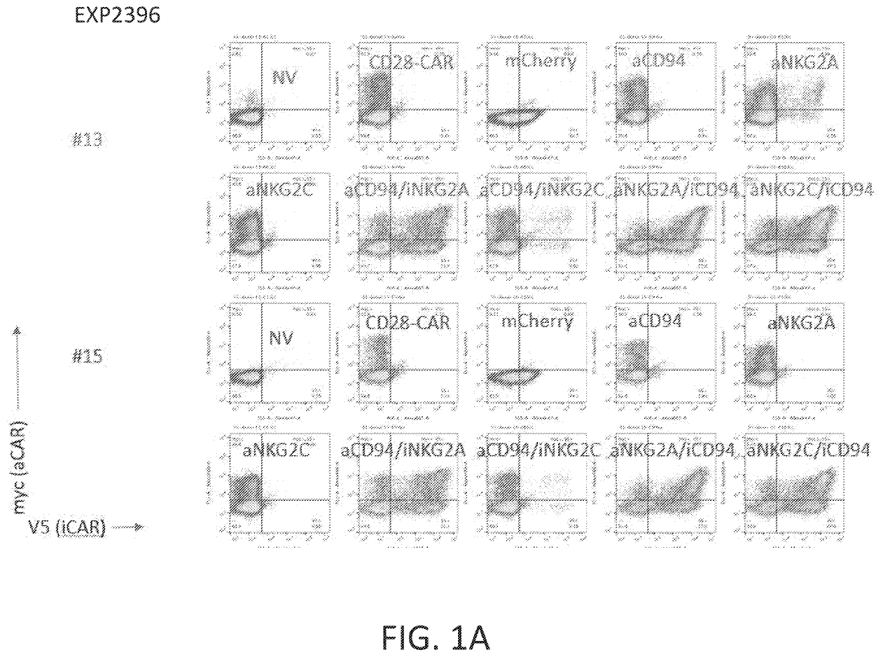

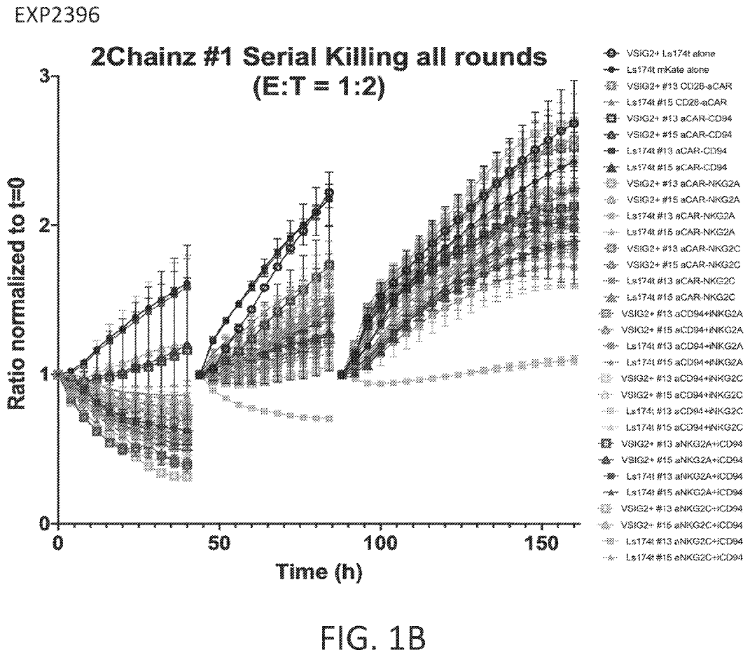

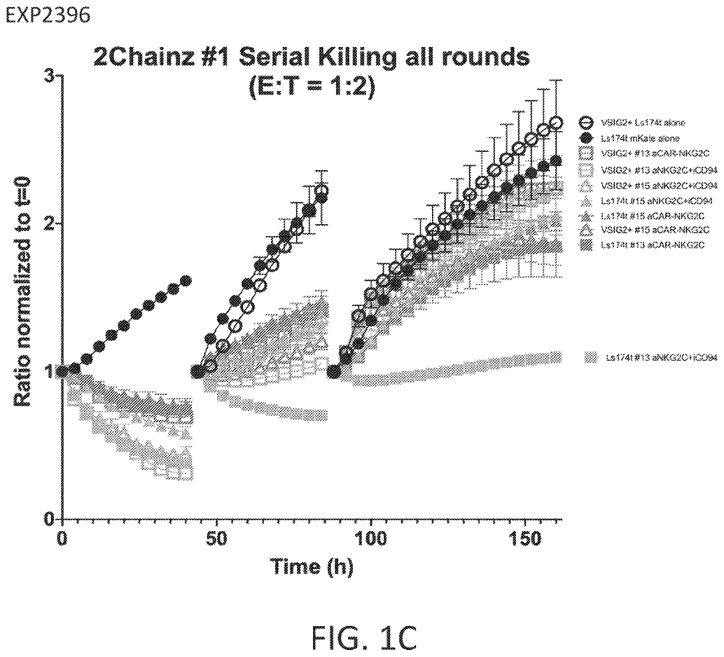

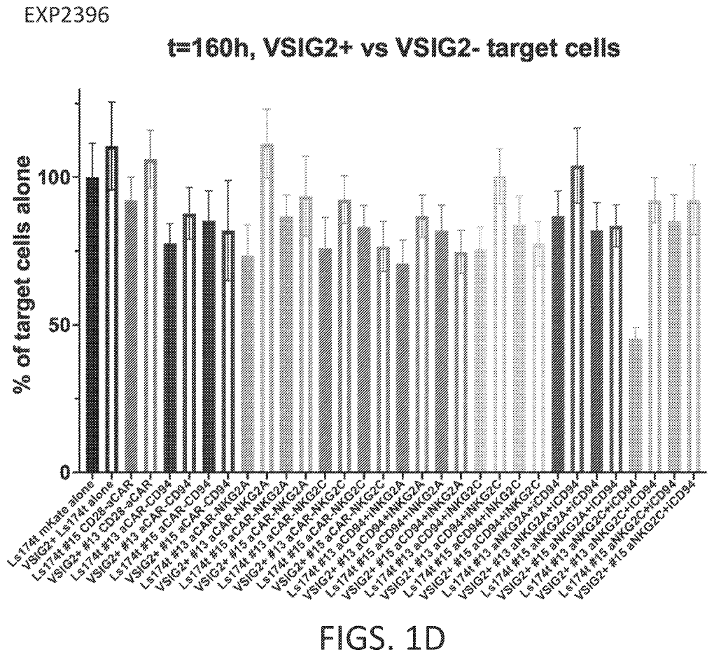

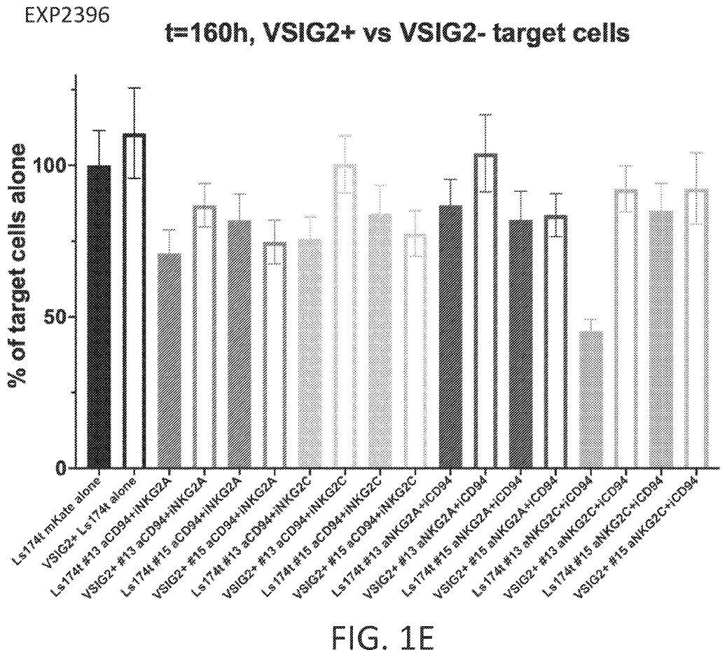

FIGS. 1A-1E provide representative data assessing the efficacy of aCAR/iCAR dimerization. FIG. 1A demonstrates successful co-transduction of the indicated aCAR/iCAR heterodimerized combinations in both NK donor cells (experiment #2396). Additionally, FIGS. 1B-1C demonstrate reductions in target cell killing when the target cells express VSIG2, indicating successful inhibition of the aCAR by the iCAR. There was a particularly significant reduction in target cell killing with the NKG2C-aCAR+CD94-iCAR combination (FIG. 1C). These data were also expressed as a percent of target cells alone (transduced only with fluorescent reporter), as shown in FIGS. 1D-1E. Results from FIG. 1E demonstrate a particularly significant inhibition of aCAR signaling by the corresponding iCAR dimerization partner using NK cells from donor #13, regardless of which dimerization domain was present in the aCAR or iCAR.

### Example 2

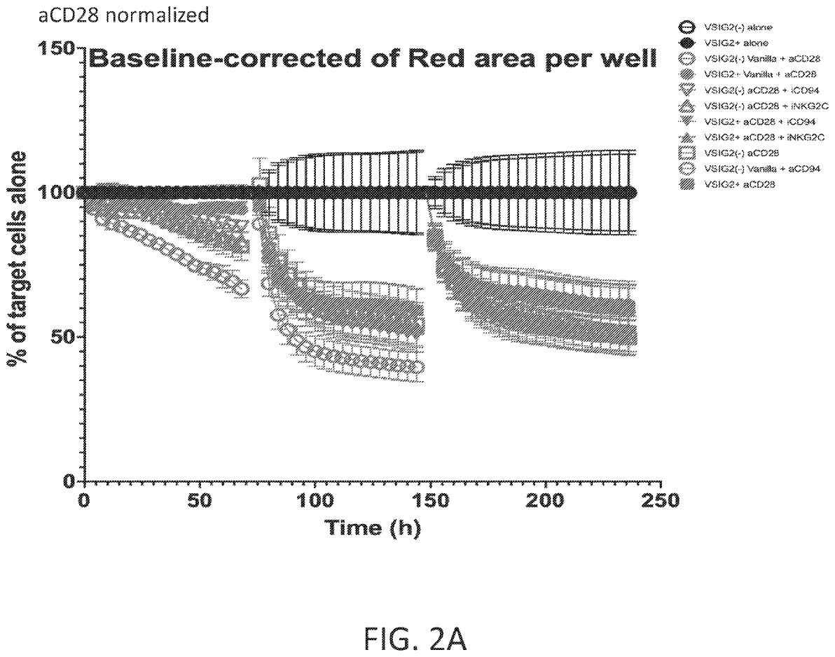

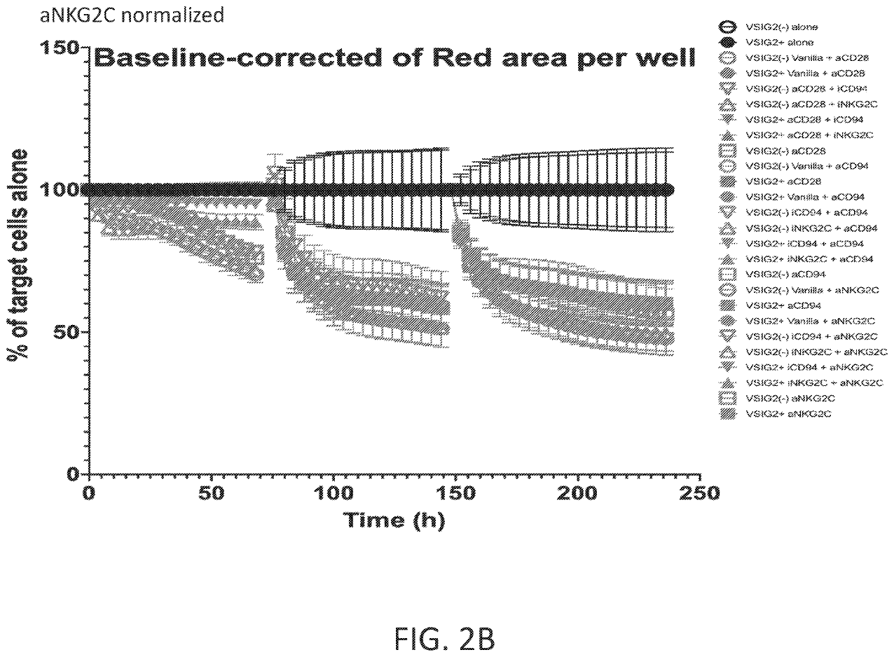

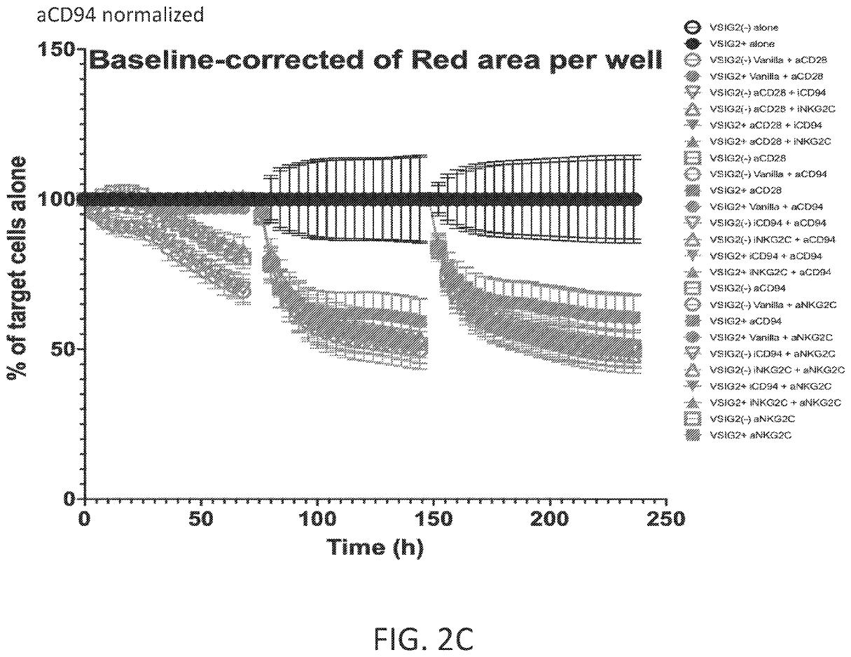

Experiments killing assays were performed using aCAR/iCAR constructs transduced with vanilla non-dimerizing CAR to serve as positive (with VSIG2) and negative (without VSIG2) controls. Using these constructs, experiments were conducted to establish a baseline level of fluorescence per well based on the percent of target cells alone, normalized to non-dimerizing CAR (FIG. 2A), NKG2C dimerization domain-containing CAR (FIG. 2B), and CD94 dimerization domain-containing CAR (FIG. 2C).

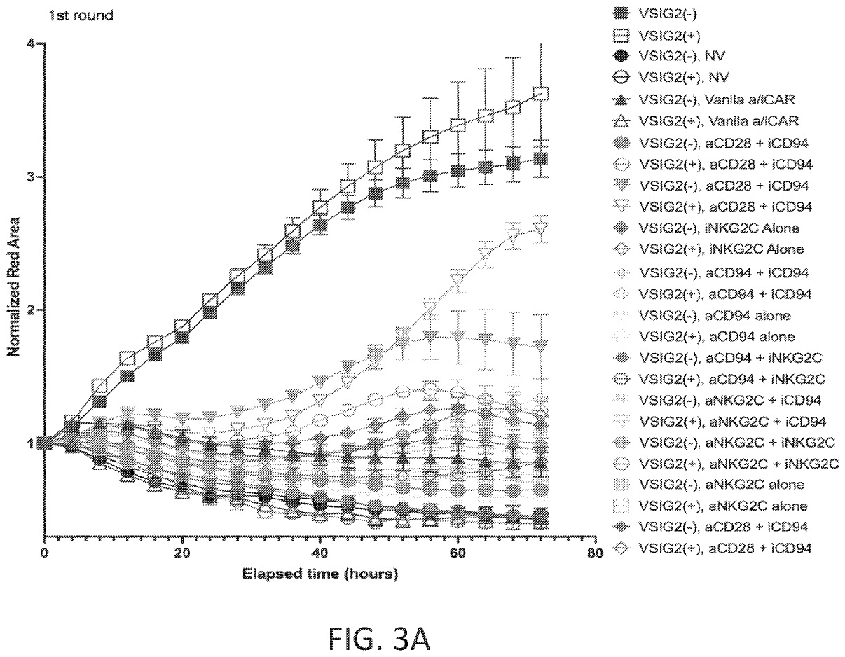

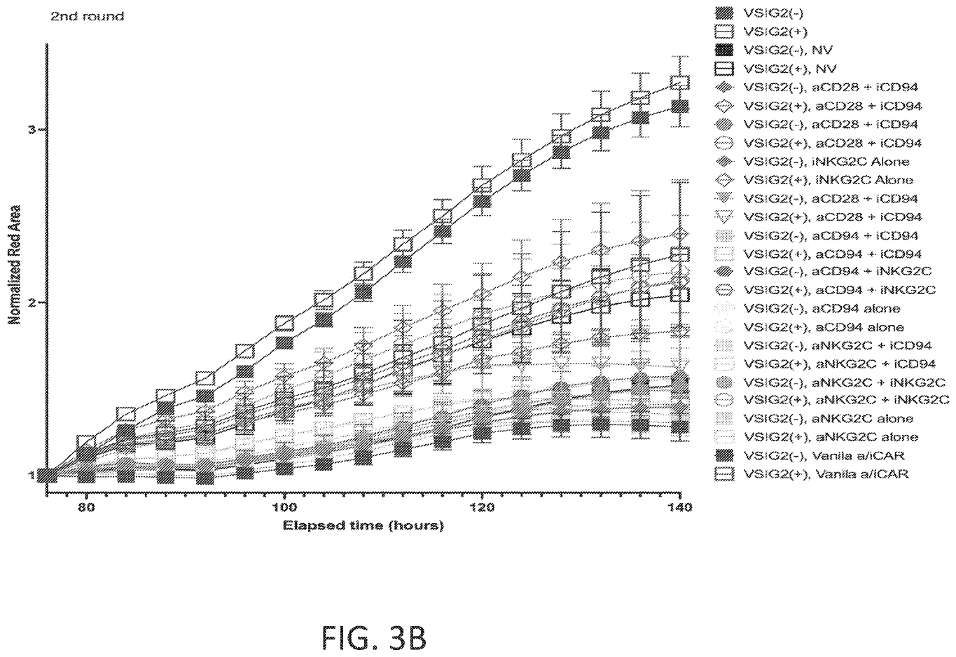

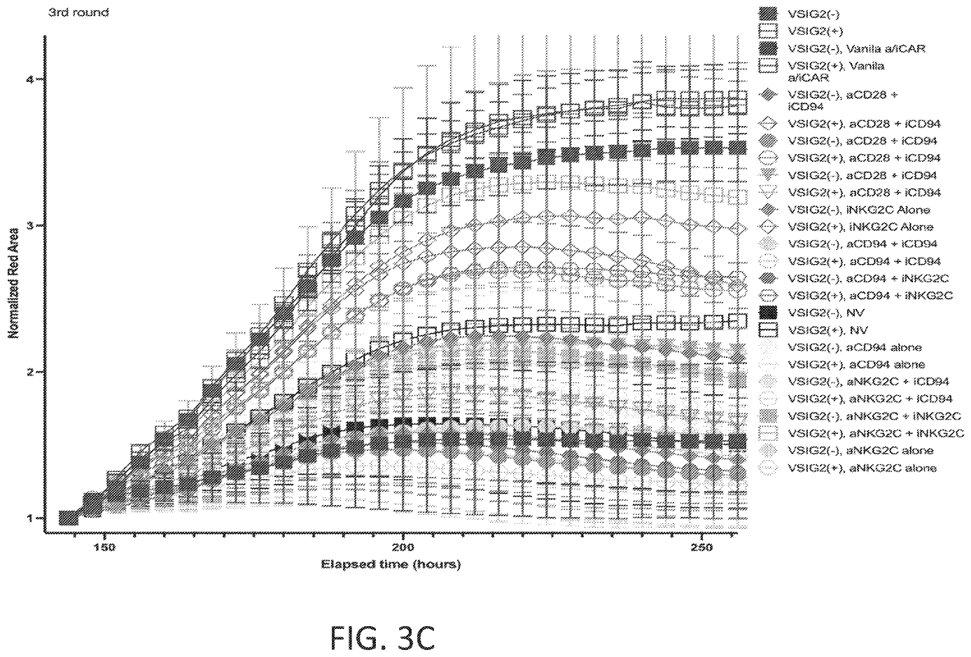

As shown in FIGS. 3A-3C, killing assays were conducted over three rounds of killing for all the indicated conditions. Target cells with VSIG2 are represented by hallowed out shapes, whereas target cells without VSIG2 are represented by solid shapes; and each color reflects the use of the same NK effector cells (with or without VSIG2-expressing target cells). Data for the first round is shown in FIG. 3A; data for the second round is shown in FIG. 3B; and data for the third round is shown in FIG. 3C. Together these data demonstrate a general trend of more fluorescence in groups with iCAR expression in the presence or VSIG2+ target cells, indicating successful aCAR/ICAR dimerization and greater iCAR inhibition across conditions.

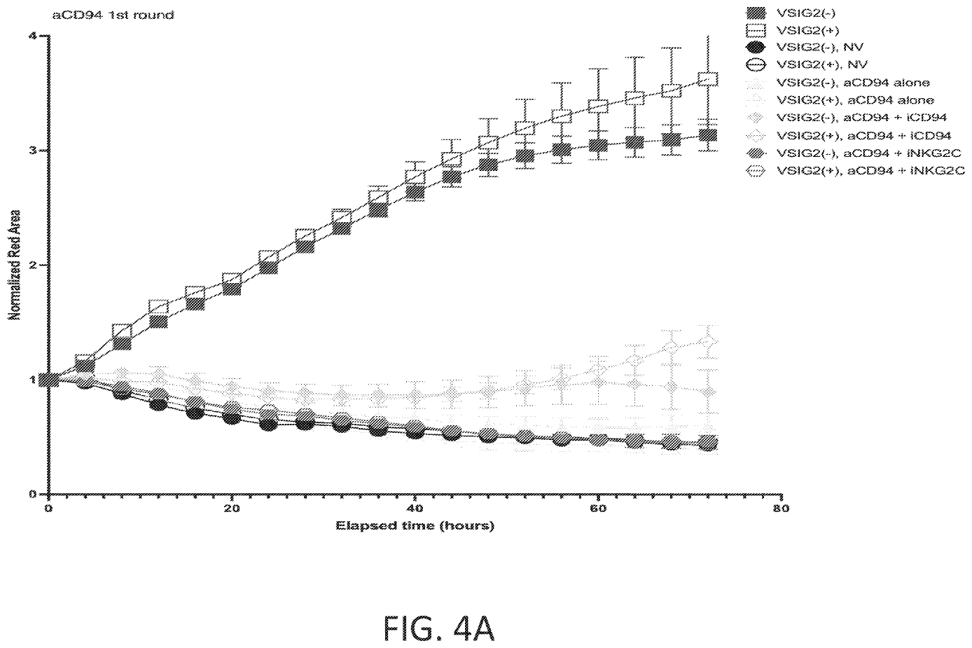

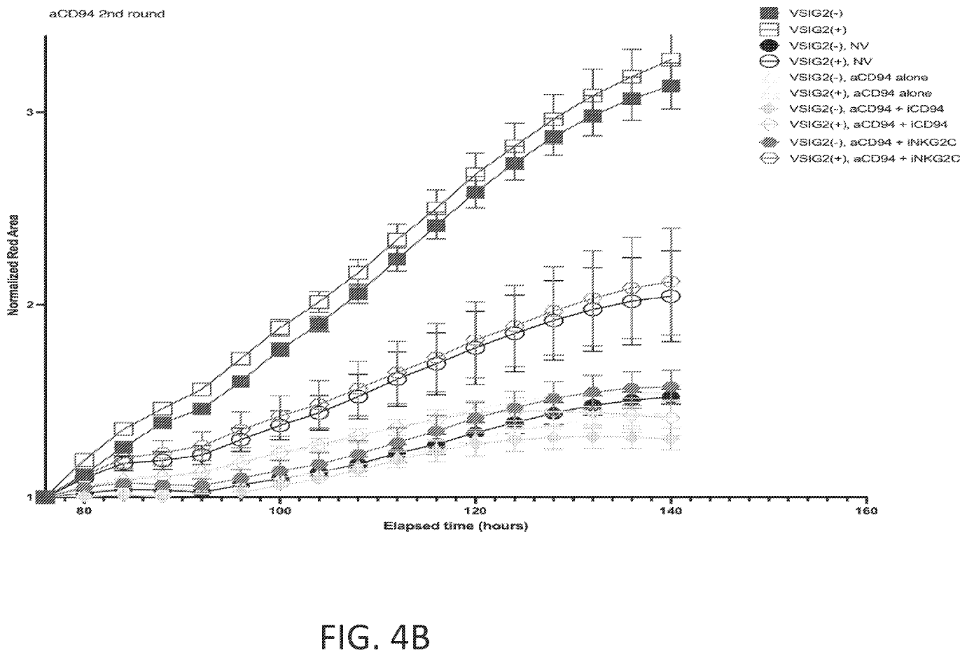

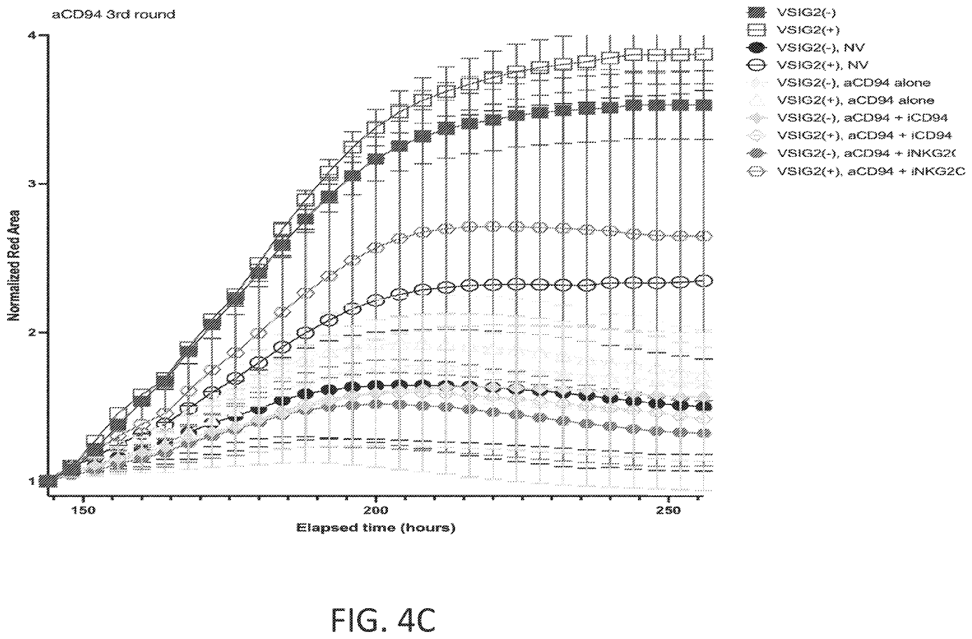

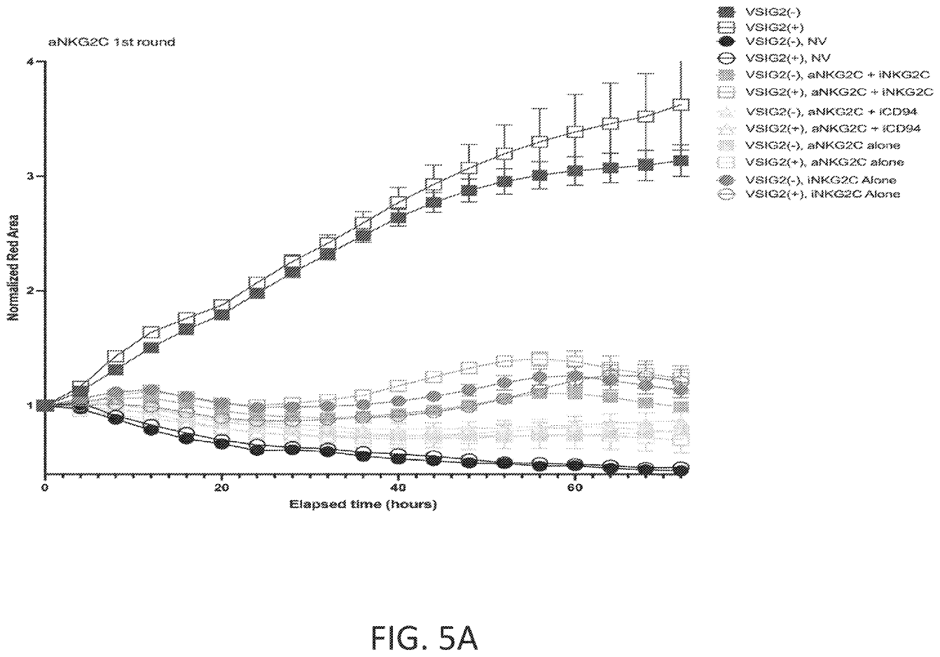

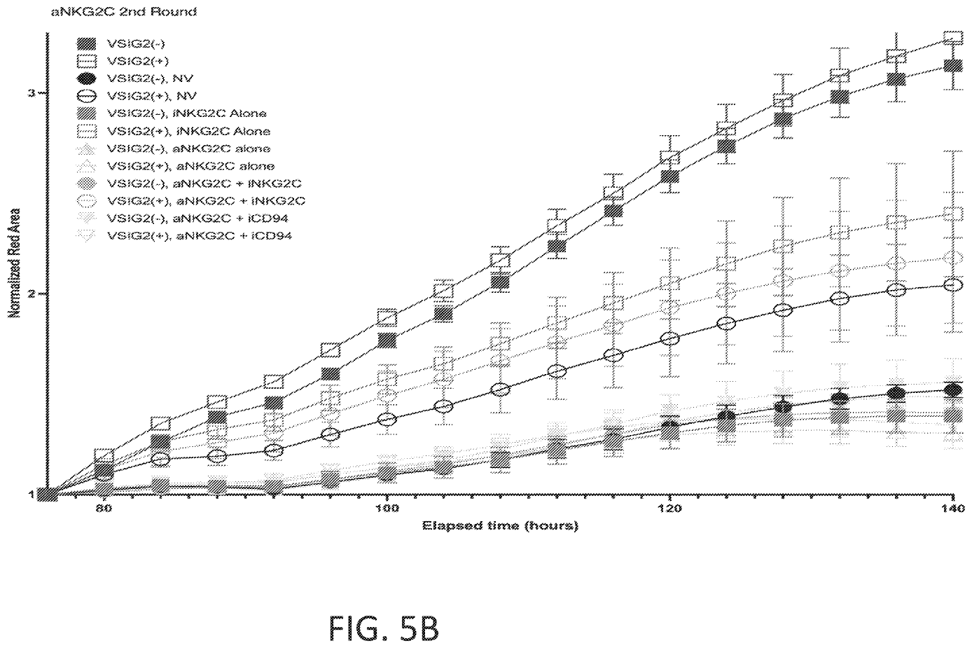

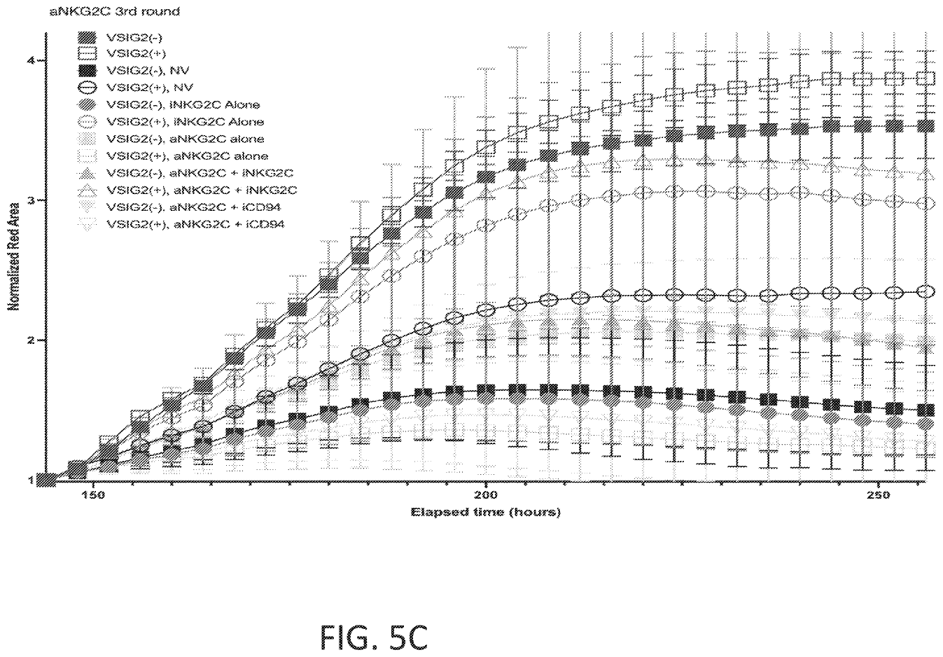

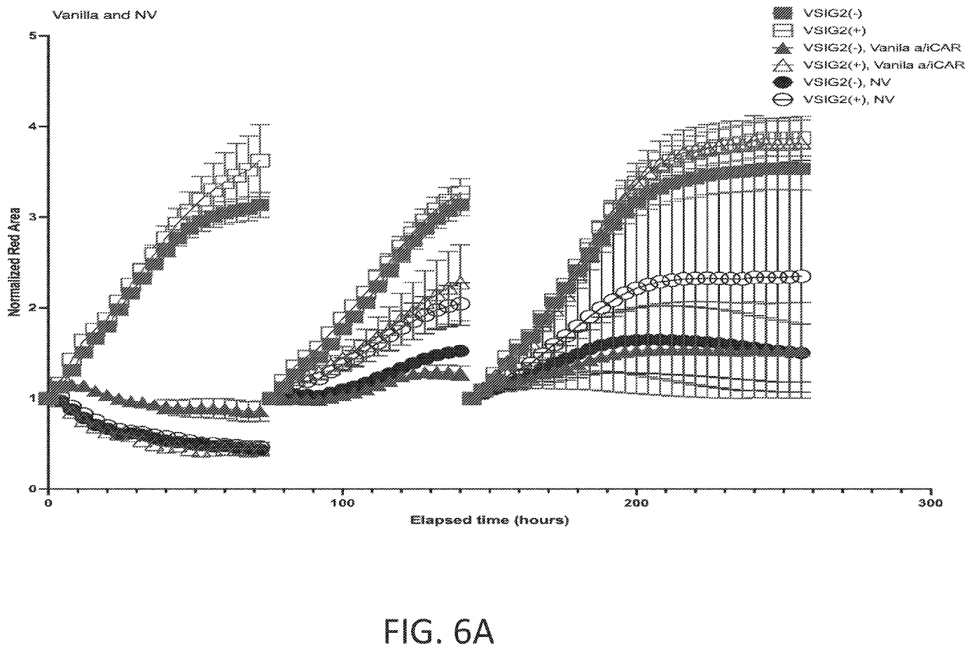

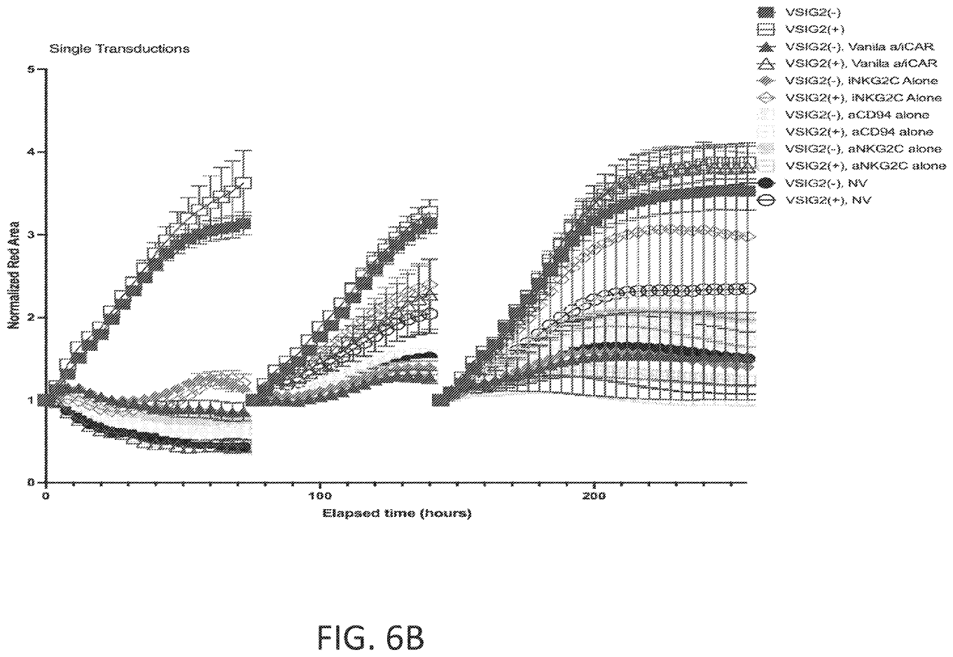

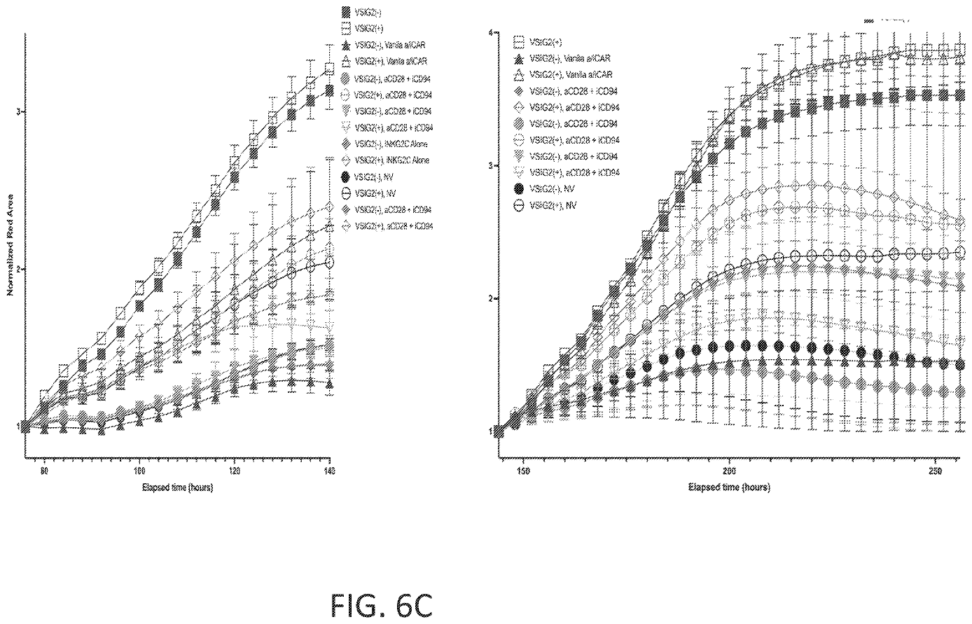

Additionally, killing assays were conducted over three rounds of killing for a subset of conditions. FIGS. 4A-4C provide representative data for test conditions involving CD94 dimerization domain-containing CAR; and FIGS. 5A-5C provide representative data for test conditions involving NKG2C dimerization domain-containing CAR. FIGS. 6A-6C provide representative data for controls (FIG. 6A: non-dimerizing CAR and no virus controls; FIG. 6B: single transduction controls; FIG. 6C: non-dimerizing CAR conditions (second and third rounds shown)).

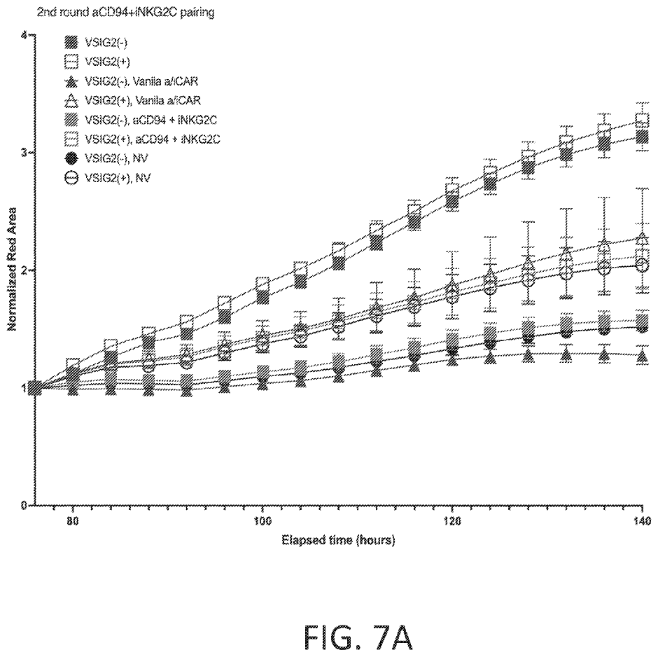

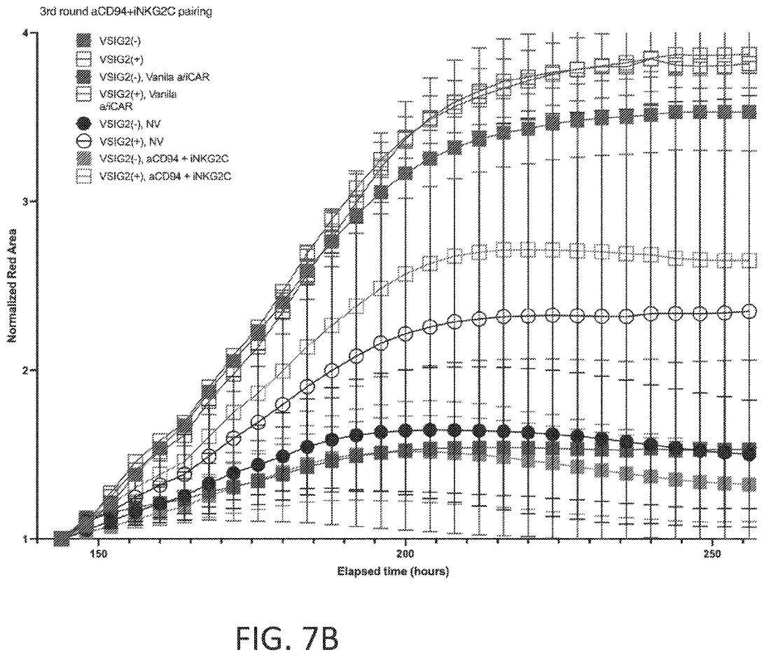

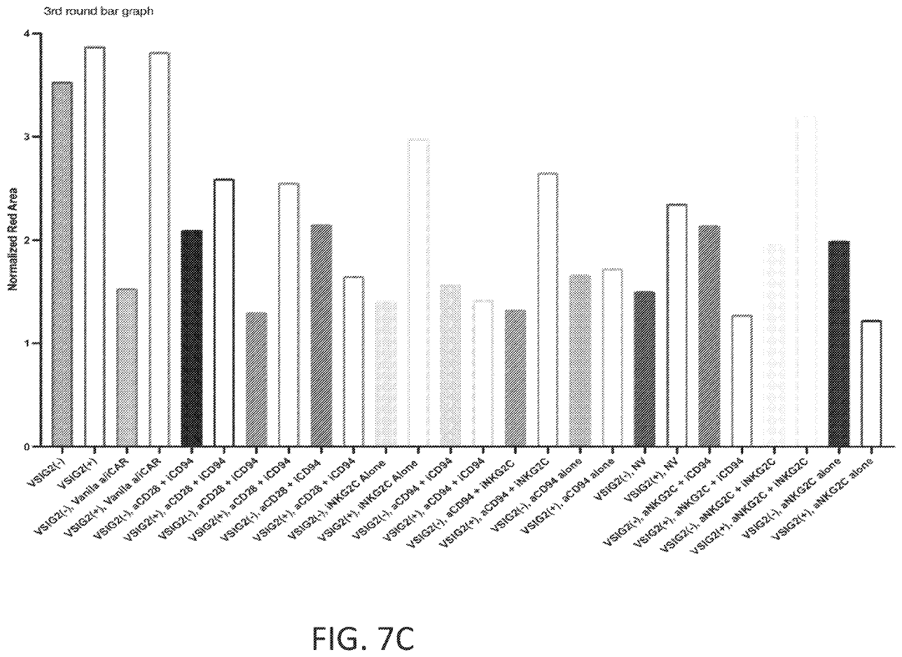

A final round of killing assays was conducted to compare CD94 dimerization domain-containing aCAR+NKG2C dimerization domain-containing iCAR pairings with non-dimerizing CAR and no virus controls; the data is provided in FIGS. 7A-7C. Data from both FIG. 7A (after two rounds of killing) and FIG. 7B (after three rounds of killing) demonstrate increased fluorescence in groups with CD94 dimerization domain-containing CAR+NKG2C dimerization domain-containing iCAR pairings in the presence or VSIG2+ target cells, indicating successful aCAR/ICAR dimerization and iCAR inhibition. FIG. 7C expresses the data from FIG. 7B in bar graph form.

Sequences. The various embodiments of the present disclosure described herein may include one or more of the sequences referenced below, which can be found in the corresponding sequence listing.

TABLE 7
Nucleic acid and amino acid sequences.

| Name | SEQ ID NO: | SEQUENCE |
|---|---|---|
| SB07524 insert | 1 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| CD94 (H) | | ATGGTATCAGCAGAAGCCTGGCAAGGCCCCTA |
| CD28 (TM) | | AGCTGCTGATCTACTGGACCAGCACCAGACACA |
| CD28 (ICD) z | | CAGGCGTGCCCAGCAGATTTTCTGGCAGCGGCT |
| | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAAAGAACAGCTTCACCAAGCTGAGCAT |
| | | CGAGCCCGCCTTCACACCCGGACCTAATATCGA |
| | | GCTGCAGAAGGACAGCGACTGCTGCAGCTGCC |
| | | AAGAGAAATGGGTCGGATTTTGGGTGCTGGTGG |
| | | TGGTTGGTGGAGTCCTGGCTTGCTATAGCTTGC |
| | | TAGTAACAGTGGCCTTTATTATTTTCTGGGTGA |
| | | GGAGTAAGAGGAGCAGGCTCCTGCACAGTGAC |
| | | TACATGAACATGACTCCCCGCCGCCCCGGGCCC |
| | | ACCCGCAAGCATTACCAGCCCTATGCCCCACCA |
| | | CGCGACTTCGCAGCCTATCGCTCCAGAGTGAAG |
| | | TTCAGCAGGAGCGCAGACGCCCCCGCGTACAA |
| | | GCAGGGCCAGAACCAGCTCTATAACGAGCTCA |
| | | ATCTAGGACGAAGAGAGGAGTACGATGTTTTG |
| | | GACAAGAGACGTGGCCGGGACCCTGAGATGGG |
| | | GGGAAAGCCGAGAAGGAAGAACCCTCAGGAAG |
| | | GCCTGTACAATGAACTGCAGAAAGATAAGATG |
| | | GCGGAGGCCTACAGTGAGATTGGGATGAAAGG |
| | | CGAGCGCCGGAGGGGCAAGGGGCACGATGGCC |
| | | TTTACCAGGGTCTCAGTACAGCCACCAAGGACA |
| | | CCTACGACGCCCTTCACATGCAGGCCCTGCCCC |
| | | CTCGC |
| SB07524 insert | 2 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| CD94 (H) | | WMSWVRQAPGKGLEWIGEIHPDSSTINYAPSLKD |
| CD28 (TM) | | RFTISRDNAKNTLFLQMDSLRPEDTGVYFCASLYF |
| CD28 (ICD) z | | GFPWFAYWGQGTPVTVSSEQKLISEEDLNGAAKN |
| | | SFTKLSIEPAFTPGPNIELQKDSDCCSCQEKWVGF |
| | | WVLVVVGGVLACYSLLVTVAFIIFWVRSKRSRLL |
| | | HSDYMNMTPRRPGPTRKHYQPYAPPRDFAAYRS |
| | | RVKFSRSADAPAYKQGQNQLYNELNLGRREEYD |
| | | VLDKRRGRDPEMGGKPRRKNPQEGLYNELQKDK |
| | | MAEAYSEIGMKGERRRGKGHDGLYQGLSTATKD |
| | | TYDALHMQALPPR |
| SB07525 insert | 3 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| CD94 (extended H) | | ATGGTATCAGCAGAAGCCTGGCAAGGCCCCTA |
| CD28 (TM) | | AGCTGCTGATCTACTGGACCAGCACCAGACACA |
| CD28 (ICD) z | | CAGGCGTGCCCAGCAGATTTTCTGGCAGCGGCT |
| | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAAAGAACAGCTTCACCAAGCTGAGCAT |
| | | CGAGCCCGCCTTCACACCCGGACCTAATATCGA |
| | | GCTGCAGAAGGACAGCGACTGCTGCAGCTGCC |
| | | AAGAGAAATGGGTCGGATACCGGTGCAACTGC |
| | | TACTTCATCAGCAGCGAGCAGAAAACCTGGTTT |
| | | TGGGTGCTGGTGGTGGTTGGTGGAGTCCTGGCT |
| | | TGCTATAGCTTGCTAGTAACAGTGGCCTTTATT |
| | | ATTTTCTGGGTGAGGAGTAAGAGGAGCAGGCTC |
| | | CTGCACAGTGACTACATGAACATGACTCCCCGC |
| | | CGCCCCGGGCCCACCCGCAAGCATTACCAGCCC |
| | | TATGCCCCACCACGCGACTTCGCAGCCTATCGC |
| | | TCCAGAGTGAAGTTCAGCAGGAGCGCAGACGC |
| | | CCCCGCGTACAAGCAGGGCCAGAACCAGCTCT |
| | | ATAACGAGCTCAATCTAGGACGAAGAGAGGAG |
| | | TACGATGTTTTGGACAAGAGACGTGGCCGGGAC |
| | | CCTGAGATGGGGGGAAAGCCGAGAAGGAAGAA |
| | | CCCTCAGGAAGGCCTGTACAATGAACTGCAGA |
| | | AAGATAAGATGGCGGAGGCCTACAGTGAGATT |
| | | GGGATGAAAGGCGAGCGCCGGAGGGGCAAGGG |
| | | GCACGATGGCCTTTACCAGGGTCTCAGTACAGC |
| | | CACCAAGGACACCTACGACGCCCTTCACATGCA |
| | | GGCCCTGCCCCCTCGC |
| SB07525 insert | 4 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| CD94 (extended H) | | WMSWVRQAPGKGLEWIGEIHPDSSTINYAPSLKD |
| CD28 (TM) | | RFTISRDNAKNTLFLQMDSLRPEDTGVYFCASLYF |
| CD28 (ICD) z | | GFPWFAYWGQGTPVTVSSEQKLISEEDLNGAAKN |
| | | SFTKLSIEPAFTPGPNIELQKDSDCCSCQEKWVGY |
| | | RCNCYFISSEQKTWFWVLVVVGGVLACYSLLVT |
| | | VAFIIFWVRSKRSRLLHSDYMNMTPRRPGPTRKH |
| | | YQPYAPPRDFAAYRSRVKFSRSADAPAYKQGQN |
| | | QLYNELNLGRREEYDVLDKRRGRDPEMGGKPRR |
| | | KNPQEGLYNELQKDKMAEAYSEIGMKGERRRGK |
| | | GHDGLYQGLSTATKDTYDALHMQALPPR |
| SB07526 insert | 5 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| NKG2A (H) | | ATGGTATCAGCAGAAGCCTGGCAAGGCCCCTA |
| CD28 (TM) | | AGCTGCTGATCTACTGGACCAGCACCAGACACA |
| CD28 (ICD) z | | CAGGCGTGCCCAGCAGATTTTCTGGCAGCGGCT |
| | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCACCCAGCACACTGATCCAGCGGCACAA |
| | | CAACAGCAGCCTGAACACCAGAACACAGAAGG |
| | | CCCGGCACTGCGGCTTTTGGGTGCTGGTGGTGG |
| | | TTGGTGGAGTCCTGGCTTGCTATAGCTTGCTAG |
| | | TAACAGTGGCCTTTATTATTTTCTGGGTGAGGA |
| | | GTAAGAGGAGCAGGCTCCTGCACAGTGACTAC |
| | | ATGAACATGACTCCCCGCCGCCCCGGGCCCACC |
| | | CGCAAGCATTACCAGCCCTATGCCCCACCACGC |
| | | GACTTCGCAGCCTATCGCTCCAGAGTGAAGTTC |
| | | AGCAGGAGCGCAGACGCCCCCGCGTACAAGCA |
| | | GGGCCAGAACCAGCTCTATAACGAGCTCAATCT |
| | | AGGACGAAGAGAGGAGTACGATGTTTTGGACA |
| | | AGAGACGTGGCCGGGACCCTGAGATGGGGGGA |
| | | AAGCCGAGAAGGAAGAACCCTCAGGAAGGCCT |
| | | GTACAATGAACTGCAGAAAGATAAGATGGCGG |
| | | AGGCCTACAGTGAGATTGGGATGAAAGGCGAG |
| | | CGCCGGAGGGGCAAGGGGCACGATGGCCTTTA |
| | | CCAGGGTCTCAGTACAGCCACCAAGGACACCTA |
| | | CGACGCCCTTCACATGCAGGCCCTGCCCCCTCG |
| | | C |
| SB07526 insert | 6 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| NKG2A (H) | | WMSWVRQAPGKGLEWIGEIHPDSSTINYAPSLKD |
| CD28 (TM) | | RFTISRDNAKNTLFLQMDSLRPEDTGVYFCASLYF |
| CD28 (ICD) z | | GFPWFAYWGQGTPVTVSSEQKLISEEDLNGAAPS |
| | | TLIQRHNNSSLNTRTQKARHCGFWVLVVVGGVL |
| | | ACYSLLVTVAFIIFWVRSKRSRLLHSDYMNMTPR |
| | | RPGPTRKHYQPYAPPRDFAAYRSRVKFSRSADAP |
| | | AYKQGQNQLYNELNLGRREEYDVLDKRRGRDPE |
| | | MGGKPRRKNPQEGLYNELQKDKMAEAYSEIGMK |
| | | GERRRGKGHDGLYQGLSTATKDTYDALHMQALP |
| | | PR |
| SB07527 insert | 7 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| NKG2C (H) | | ATGGTATCAGCAGAAGCCTGGCAAGGCCCCTA |
| CD28 (TM) | | AGCTGCTGATCTACTGGACCAGCACCAGACACA |
| CD28 (ICD) z | | CAGGCGTGCCCAGCAGATTTTCTGGCAGCGGCT |
| | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAATCCCATTCCTCGAGCAGAACAACAGC |
| | | AGCCCCAACACAAGAACCCAGAAGGCCAGACA |
| | | CTGCGGATTTTGGGTGCTGGTGGTGGTTGGTGG |
| | | AGTCCTGGCTTGCTATAGCTTGCTAGTAACAGT |
| | | GGCCTTTATTATTTTCTGGGTGAGGAGTAAGAG |
| | | GAGCAGGCTCCTGCACAGTGACTACATGAACAT |
| | | GACTCCCCGCCGCCCCGGGCCCACCCGCAAGCA |
| | | TTACCAGCCCTATGCCCCACCACGCGACTTCGC |
| | | AGCCTATCGCTCCAGAGTGAAGTTCAGCAGGAG |
| | | CGCAGACGCCCCCGCGTACAAGCAGGGCCAGA |
| | | ACCAGCTCTATAACGAGCTCAATCTAGGACGAA |
| | | GAGAGGAGTACGATGTTTTGGACAAGAGACGT |
| | | GGCCGGGACCCTGAGATGGGGGGAAAGCCGAG |
| | | AAGGAAGAACCCTCAGGAAGGCCTGTACAATG |
| | | AACTGCAGAAAGATAAGATGGCGGAGGCCTAC |
| | | AGTGAGATTGGGATGAAAGGCGAGCGCCGGAG |
| | | GGGCAAGGGGCACGATGGCCTTTACCAGGGTCT |
| | | CAGTACAGCCACCAAGGACACCTACGACGCCC |
| | | TTCACATGCAGGCCCTGCCCCCTCGC |
| SB07527 insert | 8 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| NKG2C (H) | | WMSWVRQAPGKGLEWIGEIHPDSSTINYAPSLKD |
| CD28 (TM) | | RFTISRDNAKNTLFLQMDSLRPEDTGVYFCASLYF |
| CD28 (ICD) z | | GFPWFAYWGQGTPVTVSSEQKLISEEDLNGAAIPF |
| | | LEQNNSSPNTRTQKARHCGFWVLVVVGGVLACY |
| | | SLLVTVAFIIFWVRSKRSRLLHSDYMNMTPRRPGP |
| | | TRKHYQPYAPPRDFAAYRSRVKFSRSADAPAYKQ |
| | | GQNQLYNELNLGRREEYDVLDKRRGRDPEMGGK |
| | | PRRKNPQEGLYNELQKDKMAEAYSEIGMKGERR |
| | | RGKGHDGLYQGLSTATKDTYDALHMQALPPR |
| SB07528 insert | 9 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| NKG2A (H) | | ATGGTATCAGCAGAAGCCTGGCAAGGCCCCTA |
| CD8 (H) | | AGCTGCTGATCTACTGGACCAGCACCAGACACA |
| CD28 (TM) | | CAGGCGTGCCCAGCAGATTTTCTGGCAGCGGCT |
| CD28 (ICD) z | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCACCCAGCACACTGATCCAGCGGCACAA |
| | | CAACAGCAGCCTGAACACCAGAACACAGAAGG |
| | | CCCGGCACTGCGGCACCACGACGCCAGCGCCG |
| | | CGACCACCAACACCGGCGCCCACCATCGCGTTG |
| | | CAGCCCCTGTCCCTGCGCCCAGAGGCGTGCCGG |
| | | CCAGCGGCGGGGGGCGCAGTGCACACGAGGGG |
| | | GCTGGACTTCGCCTGTGATTTTTGGGTGCTGGT |
| | | GGTGGTTGGTGGAGTCCTGGCTTGCTATAGCTT |
| | | GCTAGTAACAGTGGCCTTTATTATTTTCTGGGTG |
| | | AGGAGTAAGAGGAGCAGGCTCCTGCACAGTGA |
| | | CTACATGAACATGACTCCCCGCCGCCCCGGGCC |
| | | CACCCGCAAGCATTACCAGCCCTATGCCCCACC |
| | | ACGCGACTTCGCAGCCTATCGCTCCAGAGTGAA |
| | | GTTCAGCAGGAGCGCAGACGCCCCCGCGTACA |
| | | AGCAGGGCCAGAACCAGCTCTATAACGAGCTC |
| | | AATCTAGGACGAAGAGAGGAGTACGATGTTTT |
| | | GGACAAGAGACGTGGCCGGGACCCTGAGATGG |
| | | GGGGAAAGCCGAGAAGGAAGAACCCTCAGGAA |
| | | GGCCTGTACAATGAACTGCAGAAAGATAAGAT |
| | | GGCGGAGGCCTACAGTGAGATTGGGATGAAAG |
| | | GCGAGCGCCGGAGGGGCAAGGGGCACGATGGC |
| | | CTTTACCAGGGTCTCAGTACAGCCACCAAGGAC |
| | | ACCTACGACGCCCTTCACATGCAGGCCCTGCCC |
| | | CCTCGC |
| SB07528 insert | 10 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| NKG2A (H) | | WMSWVRQAPGKGLEWIGEIHPDSSTINYAPSLKD |
| CD8 (H) | | RFTISRDNAKNTLFLQMDSLRPEDTGVYFCASLYF |
| CD28 (TM) | | GFPWFAYWGQGTPVTVSSEQKLISEEDLNGAAPS |
| CD28 (ICD) z | | TLIQRHNNSSLNTRTQKARHCGTTTPAPRPPTPAP |
| | | TIALQPLSLRPEACRPAAGGAVHTRGLDFACDFW |
| | | VLVVVGGVLACYSLLVTVAFIIFWVRSKRSRLLHS |
| | | DYMNMTPRRPGPTRKHYQPYAPPRDFAAYRSRV |
| | | KFSRSADAPAYKQGQNQLYNELNLGRREEYDVL |
| | | DKRRGRDPEMGGKPRRKNPQEGLYNELQKDKM |
| | | AEAYSEIGMKGERRRGKGHDGLYQGLSTATKDT |
| | | YDALHMQALPPR |
| SB07529: | 11 | ATGGCCCTGCCTGTGACAGCTCTGCTGCTGCCT |
| CD8 (SS) | (nucleic acid) | CTGGCCCTGCTGCTGCATGCTGCTAGACCTGAC |
| aCEA | | ATCCAGCTGACACAGAGCCCTAGCAGCCTGTCT |
| myc | | GCCTCTGTGGGCGACAGAGTGACCATCACATGC |
| NKG2C (H) | | AAGGCCTCTCAGGACGTGGGCACAAGCGTGGC |
| CD8 (H) | | ATGGTATCAGCAGAAGCCTGGCAAGGCCCCTA |
| CD28 (TM) | | AGCTGCTGATCTACTGGACCAGCACCAGACACA |
| CD28 (ICD) z | | CAGGCGTGCCCAGCAGATTTTCTGGCAGCGGCT |
| | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAATCCCATTCCTCGAGCAGAACAACAGC |
| | | AGCCCCAACACAAGAACCCAGAAGGCCAGACA |
| | | CTGCGGAACCACGACGCCAGCGCCGCGACCAC |
| | | CAACACCGGCGCCCACCATCGCGTTGCAGCCCC |
| | | TGTCCCTGCGCCCAGAGGCGTGCCGGCCAGCGG |
| | | CGGGGGGCGCAGTGCACACGAGGGGGCTGGAC |
| | | TTCGCCTGTGATTTTTGGGTGCTGGTGGTGGTTG |
| | | GTGGAGTCCTGGCTTGCTATAGCTTGCTAGTAA |
| | | CAGTGGCCTTTATTATTTTCTGGGTGAGGAGTA |
| | | AGAGGAGCAGGCTCCTGCACAGTGACTACATG |
| | | AACATGACTCCCCGCCGCCCCGGGCCCACCCGC |
| | | AAGCATTACCAGCCCTATGCCCCACCACGCGAC |
| | | TTCGCAGCCTATCGCTCCAGAGTGAAGTTCAGC |
| | | AGGAGCGCAGACGCCCCCGCGTACAAGCAGGG |
| | | CCAGAACCAGCTCTATAACGAGCTCAATCTAGG |
| | | ACGAAGAGAGGAGTACGATGTTTTGGACAAGA |
| | | GACGTGGCCGGGACCCTGAGATGGGGGGAAAG |
| | | CCGAGAAGGAAGAACCCTCAGGAAGGCCTGTA |
| | | CAATGAACTGCAGAAAGATAAGATGGCGGAGG |
| | | CCTACAGTGAGATTGGGATGAAAGGCGAGCGC |
| | | CGGAGGGGCAAGGGGCACGATGGCCTTTACCA |
| | | GGGTCTCAGTACAGCCACCAAGGACACCTACG |
| | | ACGCCCTTCACATGCAGGCCCTGCCCCCTCGC |
| SB07529 insert | 12 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| NKG2C (H) | | WMSWVRQAPGKGLEWIGEIHPDSSTINYAPSLKD |
| CD8 (H) | | RFTISRDNAKNTLFLQMDSLRPEDTGVYFCASLYF |
| CD28 (TM) | | GFPWFAYWGQGTPVTVSSEQKLISEEDLNGAAIPF |
| CD28 (ICD) z | | LEQNNSSPNTRTQKARHCGTTTPAPRPPTPAPTIA |
| | | LQPLSLRPEACRPAAGGAVHTRGLDFACDFWVLV |
| | | VVGGVLACYSLLVTVAFIIFWVRSKRSRLLHSDY |
| | | MNMTPRRPGPTRKHYQPYAPPRDFAAYRSRVKFS |
| | | RSADAPAYKQGQNQLYNELNLGRREEYDVLDKR |
| | | RGRDPEMGGKPRRKNPQEGLYNELQKDKMAEA |
| | | YSEIGMKGERRRGKGHDGLYQGLSTATKDTYDA |
| | | LHMQALPPR |
| SB07530 insert | 13 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| CD94 (H) | | TGGTATCAGCAGAAGCAGGGCAAGTCTCCTCAG |
| LIR1 (TM) | | CTGCTGGTGTTCAACGCCGAGACACTGCCTGAA |
| LIR1 (ICD) | | GGCGTGCCCAGCAGATTTTCTGGAACAGGCAGC |
| P2A PuroR | | GGCACCCACTTCAGCCTGAGAATCAATAGCCTG |
| | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAAAGAA |
| | | CAGCTTCACCAAGCTGAGCATCGAGCCCGCCTT |
| | | CACACCCGGACCTAATATCGAGCTGCAGAAGG |
| | | ACAGCGACTGCTGCAGCTGCCAAGAGAAATGG |
| | | GTCGGAGTTATAGGGATCCTGGTGGCTGTCATA |
| | | CTCCTCTTGCTCCTCTTGTTGCTGCTTTTTTTGAT |
| | | ATTGCGCCACAGACGGCAGGGAAAGCACTGGA |
| | | CTAGTACGCAGAGGAAAGCGGACTTCCAGCAT |
| | | CCCGCAGGAGCCGTGGGGCCTGAACCCACTGAT |
| | | CGCGGCCTTCAATGGAGGTCTAGCCCGGCGGCA |
| | | GACGCACAAGAGGAAAACTTGTACGCAGCCGT |
| | | TAAGCACACCCAACCGGAGGACGGCGTTGAGA |
| | | TGGATACCCGCTCCCCTCACGATGAAGACCCTC |
| | | AAGCAGTCACTTACGCGGAAGTAAAGCATAGC |
| | | CGCCCCAGACGGGAAATGGCTAGCCCGCCGTC |
| | | CCCCCTTAGCGGGGAATTTCTGGACACTAAAGA |
| | | TAGGCAGGCGGAAGAGGACCGCCAAATGGATA |
| | | CAGAGGCGGCGGCAAGTGAAGCACCTCAAGAC |
| | | GTTACTTACGCTCAACTTCACAGCCTTACCCTCA |
| | | GGCGAGAAGCGACTGAACCACCCCCTTCCCAA |
| | | GAAGGGCCAAGCCCAGCGGTTCCTTCTATCTAT |
| | | GCTACTCTTGCTATTCACGGAAGCGGAGCTACT |
| | | AACTTCAGCCTGCTGAAGCAGGCTGGAGACGTG |
| | | GAGGAGAACCCTGGACCTATGACCGAGTACAA |
| | | GCCCACGGTGCGCCTCGCCACCCGCGACGACGT |
| | | CCCCAGGGCCGTACGCACCCTCGCCGCCGCGTT |
| | | CGCCGACTACCCCGCCACGCGCCACACCGTCGA |
| | | TCCGGACCGCCACATCGAGCGGGTCACCGAGCT |
| | | GCAAGAACTCTTCCTCACGCGCGTCGGGCTCGA |
| | | CATCGGCAAGGTGTGGGTCGCGGACGACGGCG |
| | | CCGCGGTGGCGGTCTGGACCACGCCGGAGAGC |
| | | GTCGAAGCGGGGGCGGTGTTCGCCGAGATCGG |
| | | CCCGCGCATGGCCGAGTTGAGCGGTTCCCGGCT |
| | | GGCCGCGCAGCAACAGATGGAAGGCCTCCTGG |
| | | CGCCGCACCGGCCCAAGGAGCCCGCGTGGTTCC |
| | | TGGCCACCGTCGGCGTCTCGCCCGACCACCAGG |
| | | GCAAGGGTCTGGGCAGCGCCGTCGTGCTCCCCG |
| | | GAGTGGAGGCGGCCGAGCGCGCCGGGGTGCCC |
| | | GCCTTCCTGGAGACCTCCGCGCCCCGCAACCTC |
| | | CCCTTCTACGAGCGGCTCGGCTTCACCGTCACC |
| | | GCCGACGTCGAGGTGCCCGAAGGACCGCGCAC |
| | | CTGGTGCATGACCCGCAAGCCCGGTGCC |
| SB07530 insert | 14 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| CD94 (H) | | FTFSNSGMSWVRQTPDKRLEWVASISDGGLYTHY |
| LIR1 (TM) | | PDSVKGRFTISRDNGKSTLYLQMSSLRSEDTAIYY |
| LIR1 (ICD) | | CARQGVRPFFDYWGQGTTLTVSSGKPIPNPLLGL |
| P2A PuroR | | DSTNGAAKNSFTKLSIEPAFTPGPNIELQKDSDCCS |
| | | CQEKWVGVIGILVAVILLLLLLLLLFLILRHRRQG |
| | | KHWTSTQRKADFQHPAGAVGPEPTDRGLQWRSS |
| | | PAADAQEENLYAAVKHTQPEDGVEMDTRSPHDE |
| | | DPQAVTYAEVKHSRPRREMASPPSPLSGEFLDTK |
| | | DRQAEEDRQMDTEAAASEAPQDVTYAQLHSLTL |
| | | RREATEPPPSQEGPSPAVPSIYATLAIHGSGATNFS |
| | | LLKQAGDVEENPGPMTEYKPTVRLATRDDVPRA |
| | | VRTLAAAFADYPATRHTVDPDRHIERVTELQELF |
| | | LTRVGLDIGKVWVADDGAAVAVWTTPESVEAGA |
| | | VFAEIGPRMAELSGSRLAAQQQMEGLLAPHRPKE |
| | | PAWFLATVGVSPDHQGKGLGSAVVLPGVEAAER |
| | | AGVPAFLETSAPRNLPFYERLGFTVTADVEVPEGP |
| | | RTWCMTRKPGA |
| SB07531 insert | 15 | ATGGCCTTACCAGTGACCGCCTTGCTCCT |
| sequence: | (nucleic acid) | GCCGCTGGCCTTGCTGCTCCACGCCGCCAGGCC |
| CD8 (SS) | | GGACATCCAGATGACACAGTCTCCAGCCAGCCT |
| aVSIG2 | | GTCTGCCTCTGTGGGAGAGACAGTGACCATGAC |
| V5 | | CTGTCGGGCCAGCGAGAACATCTACAGCTACCT |
| CD94 (extended H) | | GGCCTGGTATCAGCAGAAGCAGGGCAAGTCTC |
| fLIR1 (TM) | | CTCAGCTGCTGGTGTTCAACGCCGAGACACTGC |
| LIR1 (ICD) | | CTGAAGGCGTGCCCAGCAGATTTTCTGGAACAG |
| P2A PuroR | | GCAGCGGCACCCACTTCAGCCTGAGAATCAATA |
| | | GCCTGCAGCCTGAGGACTTCGGCAGCTACTACT |
| | | GCCAGCACCACTACGTGATCCCTTGGACCTTTG |
| | | GCGGAGGCACCAAGCTGGAAATCAAGGGCAGC |
| | | ACAAGCGGCTCTGGAAAACCTGGATCTGGCGA |
| | | GGGCTCTACCAAGGGCGAGGTGCAGATGGTTG |
| | | AGTCTGGCGGCGATCTGGTTAAGCCTGGCGGAA |
| | | GCCTGAAGCTGTCTTGTGCCGCCAGCGGCTTCA |
| | | CCTTCAGCAATAGCGGCATGAGCTGGGTCCGAC |
| | | AGACCCCTGACAAGAGACTGGAATGGGTCGCC |
| | | AGCATCTCTGACGGCGGCCTGTACACACACTAC |
| | | CCCGATTCTGTGAAGGGCAGATTCACCATCAGC |
| | | AGAGACAACGGCAAGAGCACCCTGTACCTGCA |
| | | GATGAGCAGCCTGAGAAGCGAGGACACCGCCA |
| | | TCTACTACTGCGCCAGACAGGGCGTCAGACCCT |
| | | TCTTCGATTATTGGGGCCAGGGCACCACACTGA |
| | | CCGTGTCATCTGGGAAGCCTATCCCGAACCCTC |
| | | TGTTGGGTCTCGATAGTACCAATGGGGCCGCAA |
| | | AGAACAGCTTCACCAAGCTGAGCATCGAGCCC |
| | | GCCTTCACACCCGGACCTAATATCGAGCTGCAG |
| | | AAGGACAGCGACTGCTGCAGCTGCCAAGAGAA |
| | | ATGGGTCGGATACCGGTGCAACTGCTACTTCAT |
| | | CAGCAGCGAGCAGAAAACCTGGGTTATAGGGA |
| | | TCCTGGTGGCTGTCATACTCCTCTTGCTCCTCTT |
| | | GTTGCTGCTTTTTTTGATATTGCGCCACAGACGG |
| | | CAGGGAAAGCACTGGACTAGTACGCAGAGGAA |
| | | AGCGGACTTCCAGCATCCCGCAGGAGCCGTGG |
| | | GGCCTGAACCCACTGATCGCGGCCTTCAATGGA |
| | | GGTCTAGCCCGGCGGCAGACGCACAAGAGGAA |
| | | AACTTGTACGCAGCCGTTAAGCACACCCAACCG |
| | | GAGGACGGCGTTGAGATGGATACCCGCTCCCCT |
| | | CACGATGAAGACCCTCAAGCAGTCACTTACGCG |
| | | GAAGTAAAGCATAGCCGCCCCAGACGGGAAAT |
| | | GGCTAGCCCGCCGTCCCCCCTTAGCGGGGAATT |
| | | TCTGGACACTAAAGATAGGCAGGCGGAAGAGG |
| | | ACCGCCAAATGGATACAGAGGCGGCGGCAAGT |
| | | GAAGCACCTCAAGACGTTACTTACGCTCAACTT |
| | | CACAGCCTTACCCTCAGGCGAGAAGCGACTGA |
| | | ACCACCCCCTTCCCAAGAAGGGCCAAGCCCAG |
| | | CGGTTCCTTCTATCTATGCTACTCTTGCTATTCA |
| | | CGGAAGCGGAGCTACTAACTTCAGCCTGCTGAA |
| | | GCAGGCTGGAGACGTGGAGGAGAACCCTGGAC |
| | | CTATGACCGAGTACAAGCCCACGGTGCGCCTCG |
| | | CCACCCGCGACGACGTCCCCAGGGCCGTACGC |
| | | ACCCTCGCCGCCGCGTTCGCCGACTACCCCGCC |
| | | ACGCGCCACACCGTCGATCCGGACCGCCACATC |
| | | GAGCGGGTCACCGAGCTGCAAGAACTCTTCCTC |
| | | ACGCGCGTCGGGCTCGACATCGGCAAGGTGTG |
| | | GGTCGCGGACGACGGCGCCGCGGTGGCGGTCT |
| | | GGACCACGCCGGAGAGCGTCGAAGCGGGGGCG |
| | | GTGTTCGCCGAGATCGGCCCGCGCATGGCCGAG |
| | | TTGAGCGGTTCCCGGCTGGCCGCGCAGCAACAG |
| | | ATGGAAGGCCTCCTGGCGCCGCACCGGCCCAA |
| | | GGAGCCCGCGTGGTTCCTGGCCACCGTCGGCGT |
| | | CTCGCCCGACCACCAGGGCAAGGGTCTGGGCA |
| | | GCGCCGTCGTGCTCCCCGGAGTGGAGGCGGCCG |
| | | AGCGCGCCGGGGTGCCCGCCTTCCTGGAGACCT |
| | | CCGCGCCCCGCAACCTCCCCTTCTACGAGCGGC |
| | | TCGGCTTCACCGTCACCGCCGACGTCGAGGTGC |
| | | CCGAAGGACCGCGCACCTGGTGCATGACCCGC |
| | | AAGCCCGGTGCC |
| SB07531 insert | 16 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| CD94 (extended H) | | FTFSNSGMSWVRQTPDKRLEWVASISDGGLYTHY |
| fLIR1 (TM) | | PDSVKGRFTISRDNGKSTLYLQMSSLRSEDTAIYY |
| LIR1 (ICD) | | CARQGVRPFFDYWGQGTTLTVSSGKPIPNPLLGL |
| P2A PuroR | | DSTNGAAKNSFTKLSIEPAFTPGPNIELQKDSDCCS |
| | | CQEKWVGYRCNCYFISSEQKTWVIGILVAVILLLL |
| | | LLLLLFLILRHRRQGKHWTSTQRKADFQHPAGAV |
| | | GPEPTDRGLQWRSSPAADAQEENLYAAVKHTQP |
| | | EDGVEMDTRSPHDEDPQAVTYAEVKHSRPRREM |
| | | ASPPSPLSGEFLDTKDRQAEEDRQMDTEAAASEA |
| | | PQDVTYAQLHSLTLRREATEPPPSQEGPSPAVPSIY |
| | | ATLAIHGSGATNFSLLKQAGDVEENPGPMTEYKP |
| | | TVRLATRDDVPRAVRTLAAAFADYPATRHTVDP |
| | | DRHIERVTELQELFLTRVGLDIGKVWVADDGAAV |
| | | AVWTTPESVEAGAVFAEIGPRMAELSGSRLAAQQ |
| | | QMEGLLAPHRPKEPAWFLATVGVSPDHQGKGLG |
| | | SAVVLPGVEAAERAGVPAFLETSAPRNLPFYERL |
| | | GFTVTADVEVPEGPRTWCMTRKPGA |
| SB07532 insert | 17 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| NKG2A (H) | | TGGTATCAGCAGAAGCAGGGCAAGTCTCCTCAG |
| LIR1 (TM) | | CTGCTGGTGTTCAACGCCGAGACACTGCCTGAA |
| LIR1 (ICD) | | GGCGTGCCCAGCAGATTTTCTGGAACAGGCAGC |
| P2A PuroR | | GGCACCCACTTCAGCCTGAGAATCAATAGCCTG |
| | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCACCCAGC |
| | | ACACTGATCCAGCGGCACAACAACAGCAGCCT |
| | | GAACACCAGAACACAGAAGGCCCGGCACTGCG |
| | | GCGTTATAGGGATCCTGGTGGCTGTCATACTCC |
| | | TCTTGCTCCTCTTGTTGCTGCTTTTTTTGATATTG |
| | | CGCCACAGACGGCAGGGAAAGCACTGGACTAG |
| | | TACGCAGAGGAAAGCGGACTTCCAGCATCCCG |
| | | CAGGAGCCGTGGGGCCTGAACCCACTGATCGC |
| | | GGCCTTCAATGGAGGTCTAGCCCGGCGGCAGAC |
| | | GCACAAGAGGAAAACTTGTACGCAGCCGTTAA |
| | | GCACACCCAACCGGAGGACGGCGTTGAGATGG |
| | | ATACCCGCTCCCCTCACGATGAAGACCCTCAAG |
| | | CAGTCACTTACGCGGAAGTAAAGCATAGCCGCC |
| | | CCAGACGGGAAATGGCTAGCCCGCCGTCCCCCC |
| | | TTAGCGGGGAATTTCTGGACACTAAAGATAGGC |
| | | AGGCGGAAGAGGACCGCCAAATGGATACAGAG |
| | | GCGGCGGCAAGTGAAGCACCTCAAGACGTTAC |
| | | TTACGCTCAACTTCACAGCCTTACCCTCAGGCG |
| | | AGAAGCGACTGAACCACCCCCTTCCCAAGAAG |
| | | GGCCAAGCCCAGCGGTTCCTTCTATCTATGCTA |
| | | CTCTTGCTATTCACGGAAGCGGAGCTACTAACT |
| | | TCAGCCTGCTGAAGCAGGCTGGAGACGTGGAG |
| | | GAGAACCCTGGACCTATGACCGAGTACAAGCC |
| | | CACGGTGCGCCTCGCCACCCGCGACGACGTCCC |
| | | CAGGGCCGTACGCACCCTCGCCGCCGCGTTCGC |
| | | CGACTACCCCGCCACGCGCCACACCGTCGATCC |
| | | GGACCGCCACATCGAGCGGGTCACCGAGCTGC |
| | | AAGAACTCTTCCTCACGCGCGTCGGGCTCGACA |
| | | TCGGCAAGGTGTGGGTCGCGGACGACGGCGCC |
| | | GCGGTGGCGGTCTGGACCACGCCGGAGAGCGT |
| | | CGAAGCGGGGGCGGTGTTCGCCGAGATCGGCC |
| | | CGCGCATGGCCGAGTTGAGCGGTTCCCGGCTGG |
| | | CCGCGCAGCAACAGATGGAAGGCCTCCTGGCG |
| | | CCGCACCGGCCCAAGGAGCCCGCGTGGTTCCTG |
| | | GCCACCGTCGGCGTCTCGCCCGACCACCAGGGC |
| | | AAGGGTCTGGGCAGCGCCGTCGTGCTCCCCGGA |
| | | GTGGAGGCGGCCGAGCGCGCCGGGGTGCCCGC |
| | | CTTCCTGGAGACCTCCGCGCCCCGCAACCTCCC |
| | | CTTCTACGAGCGGCTCGGCTTCACCGTCACCGC |
| | | CGACGTCGAGGTGCCCGAAGGACCGCGCACCT |
| | | GGTGCATGACCCGCAAGCCCGGTGCC |
| SB07532 insert | 18 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| NKG2A (H) | | FTFSNSGMSWVRQTPDKRLEWVASISDGGLYTHY |
| LIR1 (TM) | | PDSVKGRFTISRDNGKSTLYLQMSSLRSEDTAIYY |
| LIR1 (ICD) | | CARQGVRPFFDYWGQGTTLTVSSGKPIPNPLLGL |
| P2A PuroR | | DSTNGAAPSTLIQRHNNSSLNTRTQKARHCGVIGI |
| | | LVAVILLLLLLLLLFLILRHRRQGKHWTSTQRKAD |
| | | FQHPAGAVGPEPTDRGLQWRSSPAADAQEENLY |
| | | AAVKHTQPEDGVEMDTRSPHDEDPQAVTYAEVK |
| | | HSRPRREMASPPSPLSGEFLDTKDRQAEEDRQMD |
| | | TEAAASEAPQDVTYAQLHSLTLRREATEPPPSQEG |
| | | PSPAVPSIYATLAIHGSGATNFSLLKQAGDVEENP |
| | | GPMTEYKPTVRLATRDDVPRAVRTLAAAFADYP |
| | | ATRHTVDPDRHIERVTELQELFLTRVGLDIGKVW |
| | | VADDGAAVAVWTTPESVEAGAVFAEIGPRMAEL |
| | | SGSRLAAQQQMEGLLAPHRPKEPAWFLATVGVSP |
| | | DHQGKGLGSAVVLPGVEAAERAGVPAFLETSAPR |
| | | NLPFYERLGFTVTADVEVPEGPRTWCMTRKPGA |
| SB07533 insert | 19 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| NKG2C (H) | | TGGTATCAGCAGAAGCAGGGCAAGTCTCCTCAG |
| LIR1 (TM) | | CTGCTGGTGTTCAACGCCGAGACACTGCCTGAA |
| LIR1 (ICD) | | GGCGTGCCCAGCAGATTTTCTGGAACAGGCAGC |
| P2A PuroR | | GGCACCCACTTCAGCCTGAGAATCAATAGCCTG |
| | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAATCCCA |
| | | TTCCTCGAGCAGAACAACAGCAGCCCCAACAC |
| | | AAGAACCCAGAAGGCCAGACACTGCGGAGTTA |
| | | TAGGGATCCTGGTGGCTGTCATACTCCTCTTGCT |
| | | CCTCTTGTTGCTGCTTTTTTTGATATTGCGCCAC |
| | | AGACGGCAGGGAAAGCACTGGACTAGTACGCA |
| | | GAGGAAAGCGGACTTCCAGCATCCCGCAGGAG |
| | | CCGTGGGGCCTGAACCCACTGATCGCGGCCTTC |
| | | AATGGAGGTCTAGCCCGGCGGCAGACGCACAA |
| | | GAGGAAAACTTGTACGCAGCCGTTAAGCACAC |
| | | CCAACCGGAGGACGGCGTTGAGATGGATACCC |
| | | GCTCCCCTCACGATGAAGACCCTCAAGCAGTCA |
| | | CTTACGCGGAAGTAAAGCATAGCCGCCCCAGA |
| | | CGGGAAATGGCTAGCCCGCCGTCCCCCCTTAGC |
| | | GGGGAATTTCTGGACACTAAAGATAGGCAGGC |
| | | GGAAGAGGACCGCCAAATGGATACAGAGGCGG |
| | | CGGCAAGTGAAGCACCTCAAGACGTTACTTACG |
| | | CTCAACTTCACAGCCTTACCCTCAGGCGAGAAG |
| | | CGACTGAACCACCCCCTTCCCAAGAAGGGCCA |
| | | AGCCCAGCGGTTCCTTCTATCTATGCTACTCTTG |
| | | CTATTCACGGAAGCGGAGCTACTAACTTCAGCC |
| | | TGCTGAAGCAGGCTGGAGACGTGGAGGAGAAC |
| | | CCTGGACCTATGACCGAGTACAAGCCCACGGTG |
| | | CGCCTCGCCACCCGCGACGACGTCCCCAGGGCC |
| | | GTACGCACCCTCGCCGCCGCGTTCGCCGACTAC |
| | | CCCGCCACGCGCCACACCGTCGATCCGGACCGC |
| | | CACATCGAGCGGGTCACCGAGCTGCAAGAACT |
| | | CTTCCTCACGCGCGTCGGGCTCGACATCGGCAA |
| | | GGTGTGGGTCGCGGACGACGGCGCCGCGGTGG |
| | | CGGTCTGGACCACGCCGGAGAGCGTCGAAGCG |
| | | GGGGCGGTGTTCGCCGAGATCGGCCCGCGCATG |
| | | GCCGAGTTGAGCGGTTCCCGGCTGGCCGCGCAG |
| | | CAACAGATGGAAGGCCTCCTGGCGCCGCACCG |
| | | GCCCAAGGAGCCCGCGTGGTTCCTGGCCACCGT |
| | | CGGCGTCTCGCCCGACCACCAGGGCAAGGGTCT |
| | | GGGCAGCGCCGTCGTGCTCCCCGGAGTGGAGG |
| | | CGGCCGAGCGCGCCGGGGTGCCCGCCTTCCTGG |
| | | AGACCTCCGCGCCCCGCAACCTCCCCTTCTACG |
| | | AGCGGCTCGGCTTCACCGTCACCGCCGACGTCG |
| | | AGGTGCCCGAAGGACCGCGCACCTGGTGCATG |
| | | ACCCGCAAGCCCGGTGCC |
| SB07533 insert | 20 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| NKG2C (H) | | FTFSNSGMSWVRQTPDKRLEWVASISDGGLYTHY |
| LIR1 (TM) | | PDSVKGRFTISRDNGKSTLYLQMSSLRSEDTAIYY |
| LIR1 (ICD) | | CARQGVRPFFDYWGQGTTLTVSSGKPIPNPLLGL |
| P2A PuroR | | DSTNGAAIPFLEQNNSSPNTRTQKARHCGVIGILV |
| | | AVILLLLLLLLLFLILRHRRQGKHWTSTQRKADFQ |
| | | HPAGAVGPEPTDRGLQWRSSPAADAQEENLYAA |
| | | VKHTQPEDGVEMDTRSPHDEDPQAVTYAEVKHS |
| | | RPRREMASPPSPLSGEFLDTKDRQAEEDRQMDTE |
| | | AAASEAPQDVTYAQLHSLTLRREATEPPPSQEGPS |
| | | PAVPSIYATLAIHGSGATNFSLLKQAGDVEENPGP |
| | | MTEYKPTVRLATRDDVPRAVRTLAAAFADYPAT |
| | | RHTVDPDRHIERVTELQELFLTRVGLDIGKVWVA |
| | | DDGAAVAVWTTPESVEAGAVFAEIGPRMAELSGS |
| | | RLAAQQQMEGLLAPHRPKEPAWFLATVGVSPDH |
| | | QGKGLGSAVVLPGVEAAERAGVPAFLETSAPRNL |
| | | PFYERLGFTVTADVEVPEGPRTWCMTRKPGA |
| SB07534 insert | 21 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| NKG2A (H) | | TGGTATCAGCAGAAGCAGGGCAAGTCTCCTCAG |
| CD8 (H) | | CTGCTGGTGTTCAACGCCGAGACACTGCCTGAA |
| LIR1 (TM) | | GGCGTGCCCAGCAGATTTTCTGGAACAGGCAGC |
| LIR1 (ICD) | | GGCACCCACTTCAGCCTGAGAATCAATAGCCTG |
| P2A PuroR | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCACCCAGC |
| | | ACACTGATCCAGCGGCACAACAACAGCAGCCT |
| | | GAACACCAGAACACAGAAGGCCCGGCACTGCG |
| | | GCACCACGACGCCAGCGCCGCGACCACCAACA |
| | | CCGGCGCCCACCATCGCGTTGCAGCCCCTGTCC |
| | | CTGCGCCCAGAGGCGTGCCGGCCAGCGGCGGG |
| | | GGGCGCAGTGCACACGAGGGGGCTGGACTTCG |
| | | CCTGTGATGTTATAGGGATCCTGGTGGCTGTCA |
| | | TACTCCTCTTGCTCCTCTTGTTGCTGCTTTTTTTG |
| | | ATATTGCGCCACAGACGGCAGGGAAAGCACTG |
| | | GACTAGTACGCAGAGGAAAGCGGACTTCCAGC |
| | | ATCCCGCAGGAGCCGTGGGGCCTGAACCCACTG |
| | | ATCGCGGCCTTCAATGGAGGTCTAGCCCGGCGG |
| | | CAGACGCACAAGAGGAAAACTTGTACGCAGCC |
| | | GTTAAGCACACCCAACCGGAGGACGGCGTTGA |
| | | GATGGATACCCGCTCCCCTCACGATGAAGACCC |
| | | TCAAGCAGTCACTTACGCGGAAGTAAAGCATA |
| | | GCCGCCCCAGACGGGAAATGGCTAGCCCGCCG |
| | | TCCCCCCTTAGCGGGGAATTTCTGGACACTAAA |
| | | GATAGGCAGGCGGAAGAGGACCGCCAAATGGA |
| | | TACAGAGGCGGCGGCAAGTGAAGCACCTCAAG |
| | | ACGTTACTTACGCTCAACTTCACAGCCTTACCCT |
| | | CAGGCGAGAAGCGACTGAACCACCCCCTTCCC |
| | | AAGAAGGGCCAAGCCCAGCGGTTCCTTCTATCT |
| | | ATGCTACTCTTGCTATTCACGGAAGCGGAGCTA |
| | | CTAACTTCAGCCTGCTGAAGCAGGCTGGAGACG |
| | | TGGAGGAGAACCCTGGACCTATGACCGAGTAC |
| | | AAGCCCACGGTGCGCCTCGCCACCCGCGACGA |
| | | CGTCCCCAGGGCCGTACGCACCCTCGCCGCCGC |
| | | GTTCGCCGACTACCCCGCCACGCGCCACACCGT |
| | | CGATCCGGACCGCCACATCGAGCGGGTCACCG |
| | | AGCTGCAAGAACTCTTCCTCACGCGCGTCGGGC |
| | | TCGACATCGGCAAGGTGTGGGTCGCGGACGAC |
| | | GGCGCCGCGGTGGCGGTCTGGACCACGCCGGA |
| | | GAGCGTCGAAGCGGGGGCGGTGTTCGCCGAGA |
| | | TCGGCCCGCGCATGGCCGAGTTGAGCGGTTCCC |
| | | GGCTGGCCGCGCAGCAACAGATGGAAGGCCTC |
| | | CTGGCGCCGCACCGGCCCAAGGAGCCCGCGTG |
| | | GTTCCTGGCCACCGTCGGCGTCTCGCCCGACCA |
| | | CCAGGGCAAGGGTCTGGGCAGCGCCGTCGTGCT |
| | | CCCCGGAGTGGAGGCGGCCGAGCGCGCCGGGG |
| | | TGCCCGCCTTCCTGGAGACCTCCGCGCCCCGCA |
| | | ACCTCCCCTTCTACGAGCGGCTCGGCTTCACCG |
| | | TCACCGCCGACGTCGAGGTGCCCGAAGGACCG |
| | | CGCACCTGGTGCATGACCCGCAAGCCCGGTGCC |
| SB07534 insert | 22 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| NKG2A (H) | | FTFSNSGMSWVRQTPDKRLEWVASISDGGLYTHY |
| CD8 (H) | | PDSVKGRFTISRDNGKSTLYLQMSSLRSEDTAIYY |
| LIR1 (TM) | | CARQGVRPFFDYWGQGTTLTVSSGKPIPNPLLGL |
| LIR1 (ICD) | | DSTNGAAPSTLIQRHNNSSLNTRTQKARHCGTTTP |
| P2A PuroR | | APRPPTPAPTIALQPLSLRPEACRPAAGGAVHTRG |
| | | LDFACDVIGILVAVILLLLLLLLLFLILRHRRQGKH |
| | | WTSTQRKADFQHPAGAVGPEPTDRGLQWRSSPA |
| | | ADAQEENLYAAVKHTQPEDGVEMDTRSPHDEDP |
| | | QAVTYAEVKHSRPRREMASPPSPLSGEFLDTKDR |
| | | QAEEDRQMDTEAAASEAPQDVTYAQLHSLTLRR |
| | | EATEPPPSQEGPSPAVPSIYATLAIHGSGATNFSLL |
| | | KQAGDVEENPGPMTEYKPTVRLATRDDVPRAVR |
| | | TLAAAFADYPATRHTVDPDRHIERVTELQELFLTR |
| | | VGLDIGKVWVADDGAAVAVWTTPESVEAGAVF |
| | | AEIGPRMAELSGSRLAAQQQMEGLLAPHRPKEPA |
| | | WFLATVGVSPDHQGKGLGSAVVLPGVEAAERAG |
| | | VPAFLETSAPRNLPFYERLGFTVTADVEVPEGPRT |
| | | WCMTRKPGA |
| SB07535 insert | 23 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| NKG2C (H) | | TGGTATCAGCAGAAGCAGGGCAAGTCTCCTCAG |
| CD8 (H) | | CTGCTGGTGTTCAACGCCGAGACACTGCCTGAA |
| LIR1 (TM) | | GGCGTGCCCAGCAGATTTTCTGGAACAGGCAGC |
| LIR1 (ICD) | | GGCACCCACTTCAGCCTGAGAATCAATAGCCTG |
| P2A PuroR | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAATCCCA |
| | | TTCCTCGAGCAGAACAACAGCAGCCCCAACAC |
| | | AAGAACCCAGAAGGCCAGACACTGCGGAACCA |
| | | CGACGCCAGCGCCGCGACCACCAACACCGGCG |
| | | CCCACCATCGCGTTGCAGCCCCTGTCCCTGCGC |
| | | CCAGAGGCGTGCCGGCCAGCGGCGGGGGGCGC |
| | | AGTGCACACGAGGGGGCTGGACTTCGCCTGTGA |
| | | TGTTATAGGGATCCTGGTGGCTGTCATACTCCT |
| | | CTTGCTCCTCTTGTTGCTGCTTTTTTTGATATTGC |
| | | GCCACAGACGGCAGGGAAAGCACTGGACTAGT |
| | | ACGCAGAGGAAAGCGGACTTCCAGCATCCCGC |
| | | AGGAGCCGTGGGGCCTGAACCCACTGATCGCG |
| | | GCCTTCAATGGAGGTCTAGCCCGGCGGCAGACG |
| | | CACAAGAGGAAAACTTGTACGCAGCCGTTAAG |
| | | CACACCCAACCGGAGGACGGCGTTGAGATGGA |
| | | TACCCGCTCCCCTCACGATGAAGACCCTCAAGC |
| | | AGTCACTTACGCGGAAGTAAAGCATAGCCGCCC |
| | | CAGACGGGAAATGGCTAGCCCGCCGTCCCCCCT |
| | | TAGCGGGGAATTTCTGGACACTAAAGATAGGC |
| | | AGGCGGAAGAGGACCGCCAAATGGATACAGAG |
| | | GCGGCGGCAAGTGAAGCACCTCAAGACGTTAC |
| | | TTACGCTCAACTTCACAGCCTTACCCTCAGGCG |
| | | AGAAGCGACTGAACCACCCCCTTCCCAAGAAG |
| | | GGCCAAGCCCAGCGGTTCCTTCTATCTATGCTA |
| | | CTCTTGCTATTCACGGAAGCGGAGCTACTAACT |
| | | TCAGCCTGCTGAAGCAGGCTGGAGACGTGGAG |
| | | GAGAACCCTGGACCTATGACCGAGTACAAGCC |
| | | CACGGTGCGCCTCGCCACCCGCGACGACGTCCC |
| | | CAGGGCCGTACGCACCCTCGCCGCCGCGTTCGC |
| | | CGACTACCCCGCCACGCGCCACACCGTCGATCC |
| | | GGACCGCCACATCGAGCGGGTCACCGAGCTGC |
| | | AAGAACTCTTCCTCACGCGCGTCGGGCTCGACA |
| | | TCGGCAAGGTGTGGGTCGCGGACGACGGCGCC |
| | | GCGGTGGCGGTCTGGACCACGCCGGAGAGCGT |
| | | CGAAGCGGGGGCGGTGTTCGCCGAGATCGGCC |
| | | CGCGCATGGCCGAGTTGAGCGGTTCCCGGCTGG |
| | | CCGCGCAGCAACAGATGGAAGGCCTCCTGGCG |
| | | CCGCACCGGCCCAAGGAGCCCGCGTGGTTCCTG |
| | | GCCACCGTCGGCGTCTCGCCCGACCACCAGGGC |
| | | AAGGGTCTGGGCAGCGCCGTCGTGCTCCCCGGA |
| | | GTGGAGGCGGCCGAGCGCGCCGGGGTGCCCGC |
| | | CTTCCTGGAGACCTCCGCGCCCCGCAACCTCCC |
| | | CTTCTACGAGCGGCTCGGCTTCACCGTCACCGC |
| | | CGACGTCGAGGTGCCCGAAGGACCGCGCACCT |
| | | GGTGCATGACCCGCAAGCCCGGTGCC |
| SB07535 insert | 24 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| NKG2C (H) | | FTFSNSGMSWVRQTPDKRLEWVASISDGGLYTHY |
| CD8 (H) | | PDSVKGRFTISRDNGKSTLYLQMSSLRSEDTAIYY |
| LIR1 (TM) | | CARQGVRPFFDYWGQGTTLTVSSGKPIPNPLLGL |
| LIR1 (ICD) | | DSTNGAAIPFLEQNNSSPNTRTQKARHCGTTTPAP |
| P2A PuroR | | RPPTPAPTIALQPLSLRPEACRPAAGGAVHTRGLD |
| | | FACDVIGILVAVILLLLLLLLLFLILRHRRQGKHWT |
| | | STQRKADFQHPAGAVGPEPTDRGLQWRSSPAAD |
| | | AQEENLYAAVKHTQPEDGVEMDTRSPHDEDPQA |
| | | VTYAEVKHSRPRREMASPPSPLSGEFLDTKDRQA |
| | | EEDRQMDTEAAASEAPQDVTYAQLHSLTLRREAT |
| | | EPPPSQEGPSPAVPSIYATLAIHGSGATNFSLLKQA |
| | | GDVEENPGPMTEYKPTVRLATRDDVPRAVRTLA |
| | | AAFADYPATRHTVDPDRHIERVTELQELFLTRVG |
| | | LDIGKVWVADDGAAVAVWTTPESVEAGAVFAEI |
| | | GPRMAELSGSRLAAQQQMEGLLAPHRPKEPAWF |
| | | LATVGVSPDHQGKGLGSAVVLPGVEAAERAGVP |
| | | AFLETSAPRNLPFYERLGFTVTADVEVPEGPRTWC |
| | | MTRKPGA |
| SB07536 insert | 25 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| R34 (GS)3 | | ATGGTATCAGCAGAAGCCTGGCAAGGCCCCTA |
| CD8 (H) | | AGCTGCTGATCTACTGGACCAGCACCAGACACA |
| CD28 (TM) | | CAGGCGTGCCCAGCAGATTTTCTGGCAGCGGCT |
| CD28 (ICD) z | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCACTGGAAATCAGGGCCGCCTTCCTGGAA |
| | | AAAGAGAACACAGCCCTGCGGACAAGAGCCGC |
| | | CGAGCTGAGAAAACGCGTGGGCAGATGCCGGA |
| | | ACATCGTGTCTAAGTACGAGACAAGATACGGCC |
| | | CTCTGGGCTCTGGTAGTGGATCTACCACGACGC |
| | | CAGCGCCGCGACCACCAACACCGGCGCCCACC |
| | | ATCGCGTTGCAGCCCCTGTCCCTGCGCCCAGAG |
| | | GCGTGCCGGCCAGCGGCGGGGGGCGCAGTGCA |
| | | CACGAGGGGGCTGGACTTCGCCTGTGATTTTTG |
| | | GGTGCTGGTGGTGGTTGGTGGAGTCCTGGCTTG |
| | | CTATAGCTTGCTAGTAACAGTGGCCTTTATTATT |
| | | TTCTGGGTGAGGAGTAAGAGGAGCAGGCTCCT |
| | | GCACAGTGACTACATGAACATGACTCCCCGCCG |
| | | CCCCGGGCCCACCCGCAAGCATTACCAGCCCTA |
| | | TGCCCCACCACGCGACTTCGCAGCCTATCGCTC |
| | | CAGAGTGAAGTTCAGCAGGAGCGCAGACGCCC |
| | | CCGCGTACAAGCAGGGCCAGAACCAGCTCTAT |
| | | AACGAGCTCAATCTAGGACGAAGAGAGGAGTA |
| | | CGATGTTTTGGACAAGAGACGTGGCCGGGACCC |
| | | TGAGATGGGGGGAAAGCCGAGAAGGAAGAACC |
| | | CTCAGGAAGGCCTGTACAATGAACTGCAGAAA |
| | | GATAAGATGGCGGAGGCCTACAGTGAGATTGG |
| | | GATGAAAGGCGAGCGCCGGAGGGGCAAGGGGC |
| | | ACGATGGCCTTTACCAGGGTCTCAGTACAGCCA |
| | | CCAAGGACACCTACGACGCCCTTCACATGCAGG |
| | | CCCTGCCCCCTCGC |
| SB07536 insert | 26 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| R34 (GS)3 | | WMSWVRQAPGKGLEWIGEIHPDSSTINYAPSLKD |
| CD8 (H) | | RFTISRDNAKNTLFLQMDSLRPEDTGVYFCASLYF |
| CD28 (TM) | | GFPWFAYWGQGTPVTVSSEQKLISEEDLNGAALE |
| CD28 (ICD) z | | IRAAFLEKENTALRTRAAELRKRVGRCRNIVSKYE |
| | | TRYGPLGSGSGSTTTPAPRPPTPAPTIALQPLSLRP |
| | | EACRPAAGGAVHTRGLDFACDFWVLVVVGGVL |
| | | ACYSLLVTVAFIIFWVRSKRSRLLHSDYMNMTPR |
| | | RPGPTRKHYQPYAPPRDFAAYRSRVKFSRSADAP |
| | | AYKQGQNQLYNELNLGRREEYDVLDKRRGRDPE |
| | | MGGKPRRKNPQEGLYNELQKDKMAEAYSEIGMK |
| | | GERRRGKGHDGLYQGLSTATKDTYDALHMQALP |
| | | PR |
| SB07537 insert | 27 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| E34-I (GS)3 | | ATGGTATCAGCAGAAGCCTGGCAAGGCCCCTA |
| CD8 (H) | | AGCTGCTGATCTACTGGACCAGCACCAGACACA |
| CD28 (TM) | | CAGGCGTGCCCAGCAGATTTTCTGGCAGCGGCT |
| CD28 (ICD) z | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAATTACAATTAGAGCCGCATTCCTCGAG |
| | | AAAGAAAACACCGCTCTGAGAACCGAGATTGC |
| | | CGAACTCGAAAAAGAAGTGGGGCGCTGCGAGA |
| | | ATATCGTGTCCAAATACGAAACCCGCTACGGCC |
| | | CACTCGGCTCTGGTAGTGGATCTACCACGACGC |
| | | CAGCGCCGCGACCACCAACACCGGCGCCCACC |
| | | ATCGCGTTGCAGCCCCTGTCCCTGCGCCCAGAG |
| | | GCGTGCCGGCCAGCGGCGGGGGGCGCAGTGCA |
| | | CACGAGGGGGCTGGACTTCGCCTGTGATTTTTG |
| | | GGTGCTGGTGGTGGTTGGTGGAGTCCTGGCTTG |
| | | CTATAGCTTGCTAGTAACAGTGGCCTTTATTATT |
| | | TTCTGGGTGAGGAGTAAGAGGAGCAGGCTCCT |
| | | GCACAGTGACTACATGAACATGACTCCCCGCCG |
| | | CCCCGGGCCCACCCGCAAGCATTACCAGCCCTA |
| | | TGCCCCACCACGCGACTTCGCAGCCTATCGCTC |
| | | CAGAGTGAAGTTCAGCAGGAGCGCAGACGCCC |
| | | CCGCGTACAAGCAGGGCCAGAACCAGCTCTAT |
| | | AACGAGCTCAATCTAGGACGAAGAGAGGAGTA |
| | | CGATGTTTTGGACAAGAGACGTGGCCGGGACCC |
| | | TGAGATGGGGGGAAAGCCGAGAAGGAAGAACC |
| | | CTCAGGAAGGCCTGTACAATGAACTGCAGAAA |
| | | GATAAGATGGCGGAGGCCTACAGTGAGATTGG |
| | | GATGAAAGGCGAGCGCCGGAGGGGCAAGGGGC |
| | | ACGATGGCCTTTACCAGGGTCTCAGTACAGCCA |
| | | CCAAGGACACCTACGACGCCCTTCACATGCAGG |
| | | CCCTGCCCCCTCGC |
| SB07537 insert | 28 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| E34-I (GS)3 | | WMSWVRQAPGKGLEWIGEIHPDSSTINYAPSLKD |
| CD8 (H) | | RFTISRDNAKNTLFLQMDSLRPEDTGVYFCASLYF |
| CD28 (TM) | | GFPWFAYWGQGTPVTVSSEQKLISEEDLNGAAITI |
| CD28 (ICD) 2 | | RAAFLEKENTALRTEIAELEKEVGRCENIVSKYET |
| | | RYGPLGSGSGSTTTPAPRPPTPAPTIALQPLSLRPE |
| | | ACRPAAGGAVHTRGLDFACDFWVLVVVGGVLA |
| | | CYSLLVTVAFIIFWVRSKRSRLLHSDYMNMTPRR |
| | | PGPTRKHYQPYAPPRDFAAYRSRVKFSRSADAPA |
| | | YKQGQNQLYNELNLGRREEYDVLDKRRGRDPEM |
| | | GGKPRRKNPQEGLYNELQKDKMAEAYSEIGMKG |
| | | ERRRGKGHDGLYQGLSTATKDTYDALHMQALPP |
| | | R |
| SB07538 insert | 29 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| E34-V (GS)3 | | ATGGTATCAGCAGAAGCCTGGCAAGGCCCCTA |
| CD8 (H) | | AGCTGCTGATCTACTGGACCAGCACCAGACACA |
| CD28 (TM) | | CAGGCGTGCCCAGCAGATTTTCTGGCAGCGGCT |
| CD28 (ICD) z | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAATTACAATTAGAGCCGCCTTCCTGGAA |
| | | AAAGAGAACACAGCCCTGAGAACCGAGGTGGC |
| | | CGAACTCGAGAAAGAAGTGGGCCGCTGCGAGA |
| | | ACATCGTGTCTAAGTACGAGACAAGATACGGG |
| | | CCCCTGGGCAGCGGTAGCGGCTCCACCACGAC |
| | | GCCAGCGCCGCGACCACCAACACCGGCGCCCA |
| | | CCATCGCGTTGCAGCCCCTGTCCCTGCGCCCAG |
| | | AGGCGTGCCGGCCAGCGGGGGGGGCGCAGTG |
| | | CACACGAGGGGGCTGGACTTCGCCTGTGATTTT |
| | | TGGGTGCTGGTGGTGGTTGGTGGAGTCCTGGCT |
| | | TGCTATAGCTTGCTAGTAACAGTGGCCTTTATT |
| | | ATTTTCTGGGTGAGGAGTAAGAGGAGCAGGCTC |
| | | CTGCACAGTGACTACATGAACATGACTCCCCGC |
| | | CGCCCCGGGCCCACCCGCAAGCATTACCAGCCC |
| | | TATGCCCCACCACGCGACTTCGCAGCCTATCGC |
| | | TCCAGAGTGAAGTTCAGCAGGAGCGCAGACGC |
| | | CCCCGCGTACAAGCAGGGCCAGAACCAGCTCT |
| | | ATAACGAGCTCAATCTAGGACGAAGAGAGGAG |
| | | TACGATGTTTTGGACAAGAGACGTGGCCGGGAC |
| | | CCTGAGATGGGGGGAAAGCCGAGAAGGAAGAA |
| | | CCCTCAGGAAGGCCTGTACAATGAACTGCAGA |
| | | AAGATAAGATGGCGGAGGCCTACAGTGAGATT |
| | | GGGATGAAAGGCGAGCGCCGGAGGGGCAAGGG |
| | | GCACGATGGCCTTTACCAGGGTCTCAGTACAGC |
| | | CACCAAGGACACCTACGACGCCCTTCACATGCA |
| | | GGCCCTGCCCCCTCGC |
| SB07538 insert | 30 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| E34-V (GS)3 | | WMSWVRQAPGKGLEWIGEIHPDSSTINYAPSLKD |
| CD8 (H) | | RFTISRDNAKNTLFLQMDSLRPEDTGVYFCASLYF |
| CD28 (TM) | | GFPWFAYWGQGTPVTVSSEQKLISEEDLNGAAITI |
| CD28 (ICD) z | | RAAFLEKENTALRTEVAELEKEVGRCENIVSKYE |
| | | TRYGPLGSGSGSTTTPAPRPPTPAPTIALQPLSLRP |
| | | EACRPAAGGAVHTRGLDFACDFWVLVVVGGVL |
| | | ACYSLLVTVAFIIFWVRSKRSRLLHSDYMNMTPR |
| | | RPGPTRKHYQPYAPPRDFAAYRSRVKFSRSADAP |
| | | AYKQGQNQLYNELNLGRREEYDVLDKRRGRDPE |
| | | MGGKPRRKNPQEGLYNELQKDKMAEAYSEIGMK |
| | | GERRRGKGHDGLYQGLSTATKDTYDALHMQALP |
| | | PR |
| SB07539 insert | 31 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| E34-N (GS)3 | | ATGGTATCAGCAGAAGCCTGGCAAGGCCCCTA |
| CD8 (H) | | AGCTGCTGATCTACTGGACCAGCACCAGACACA |
| CD28 (TM) | | CAGGCGTGCCCAGCAGATTTTCTGGCAGCGGCT |
| CD28 (ICD) z | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAATTACAATCAGAGCCGCCTTCCTCGAG |
| | | AAAGAGAACACAGCCCTGAGAACCGAGAACGC |
| | | CGAGCTGGAAAAAGAAGTGGGCCGCTGCGAGA |
| | | ACATCGTGTCTAAGTACGAGACAAGATACGGCC |
| | | CACTCGGCTCCGGATCTGGAAGCACCACGACGC |
| | | CAGCGCCGCGACCACCAACACCGGCGCCCACC |
| | | ATCGCGTTGCAGCCCCTGTCCCTGCGCCCAGAG |
| | | GCGTGCCGGCCAGCGGCGGGGGGCGCAGTGCA |
| | | CACGAGGGGGCTGGACTTCGCCTGTGATTTTTG |
| | | GGTGCTGGTGGTGGTTGGTGGAGTCCTGGCTTG |
| | | CTATAGCTTGCTAGTAACAGTGGCCTTTATTATT |
| | | TTCTGGGTGAGGAGTAAGAGGAGCAGGCTCCT |
| | | GCACAGTGACTACATGAACATGACTCCCCGCCG |
| | | CCCCGGGCCCACCCGCAAGCATTACCAGCCCTA |
| | | TGCCCCACCACGCGACTTCGCAGCCTATCGCTC |
| | | CAGAGTGAAGTTCAGCAGGAGCGCAGACGCCC |
| | | CCGCGTACAAGCAGGGCCAGAACCAGCTCTAT |
| | | AACGAGCTCAATCTAGGACGAAGAGAGGAGTA |
| | | CGATGTTTTGGACAAGAGACGTGGCCGGGACCC |
| | | TGAGATGGGGGGAAAGCCGAGAAGGAAGAACC |
| | | CTCAGGAAGGCCTGTACAATGAACTGCAGAAA |
| | | GATAAGATGGCGGAGGCCTACAGTGAGATTGG |
| | | GATGAAAGGCGAGCGCCGGAGGGGCAAGGGGC |
| | | ACGATGGCCTTTACCAGGGTCTCAGTACAGCCA |
| | | CCAAGGACACCTACGACGCCCTTCACATGCAGG |
| | | CCCTGCCCCCTCGC |
| SB07539 insert | 32 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| E34-N (GS)3 | | WMSWVRQAPGKGLEWIGEIHPDSSTINYAPSLKD |
| CD8 (H) | | RFTISRDNAKNTLFLQMDSLRPEDTGVYFCASLYF |
| CD28 (TM) | | GFPWFAYWGQGTPVTVSSEQKLISEEDLNGAAITI |
| CD28 (ICD) z | | RAAFLEKENTALRTENAELEKEVGRCENIVSKYE |
| | | TRYGPLGSGSGSTTTPAPRPPTPAPTIALQPLSLRP |
| | | EACRPAAGGAVHTRGLDFACDFWVLVVVGGVL |
| | | ACYSLLVTVAFIIFWVRSKRSRLLHSDYMNMTPR |
| | | RPGPTRKHYQPYAPPRDFAAYRSRVKFSRSADAP |
| | | AYKQGQNQLYNELNLGRREEYDVLDKRRGRDPE |
| | | MGGKPRRKNPQEGLYNELQKDKMAEAYSEIGMK |
| | | GERRRGKGHDGLYQGLSTATKDTYDALHMQALP |
| | | PR |
| SB07540 insert | 33 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| R34 (GS)3 | | TGGTATCAGCAGAAGCAGGGCAAGTCTCCTCAG |
| CD8 (H) | | CTGCTGGTGTTCAACGCCGAGACACTGCCTGAA |
| LIR1 (TM) | | GGCGTGCCCAGCAGATTTTCTGGAACAGGCAGC |
| LIR1 (ICD) | | GGCACCCACTTCAGCCTGAGAATCAATAGCCTG |
| P2A PuroR | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCACTGGAA |
| | | ATCAGGGCCGCCTTCCTGGAAAAAGAGAACAC |
| | | AGCCCTGCGGACAAGAGCCGCCGAGCTGAGAA |
| | | AACGCGTGGGCAGATGCCGGAACATCGTGTCTA |
| | | AGTACGAGACAAGATACGGCCCTCTGGGCTCTG |
| | | GTAGTGGATCTACCACGACGCCAGCGCCGCGAC |
| | | CACCAACACCGGCGCCCACCATCGCGTTGCAGC |
| | | CCCTGTCCCTGCGCCCAGAGGCGTGCCGGCCAG |
| | | CGGCGGGGGGCGCAGTGCACACGAGGGGGCTG |
| | | GACTTCGCCTGTGATGTTATAGGGATCCTGGTG |
| | | GCTGTCATACTCCTCTTGCTCCTCTTGTTGCTGC |
| | | TTTTTTTGATATTGCGCCACAGACGGCAGGGAA |
| | | AGCACTGGACTAGTACGCAGAGGAAAGCGGAC |
| | | TTCCAGCATCCCGCAGGAGCCGTGGGGCCTGAA |
| | | CCCACTGATCGCGGCCTTCAATGGAGGTCTAGC |
| | | CCGGCGGCAGACGCACAAGAGGAAAACTTGTA |
| | | CGCAGCCGTTAAGCACACCCAACCGGAGGACG |
| | | GCGTTGAGATGGATACCCGCTCCCCTCACGATG |
| | | AAGACCCTCAAGCAGTCACTTACGCGGAAGTA |
| | | AAGCATAGCCGCCCCAGACGGGAAATGGCTAG |
| | | CCCGCCGTCCCCCCTTAGCGGGGAATTTCTGGA |
| | | CACTAAAGATAGGCAGGCGGAAGAGGACCGCC |
| | | AAATGGATACAGAGGCGGCGGCAAGTGAAGCA |
| | | CCTCAAGACGTTACTTACGCTCAACTTCACAGC |
| | | CTTACCCTCAGGCGAGAAGCGACTGAACCACCC |
| | | CCTTCCCAAGAAGGGCCAAGCCCAGCGGTTCCT |
| | | TCTATCTATGCTACTCTTGCTATTCACGGAAGCG |
| | | GAGCTACTAACTTCAGCCTGCTGAAGCAGGCTG |
| | | GAGACGTGGAGGAGAACCCTGGACCTATGACC |
| | | GAGTACAAGCCCACGGTGCGCCTCGCCACCCGC |
| | | GACGACGTCCCCAGGGCCGTACGCACCCTCGCC |
| | | GCCGCGTTCGCCGACTACCCCGCCACGCGCCAC |
| | | ACCGTCGATCCGGACCGCCACATCGAGCGGGTC |
| | | ACCGAGCTGCAAGAACTCTTCCTCACGCGCGTC |
| | | GGGCTCGACATCGGCAAGGTGTGGGTCGCGGA |
| | | CGACGGCGCCGCGGTGGCGGTCTGGACCACGC |
| | | CGGAGAGCGTCGAAGCGGGGGCGGTGTTCGCC |
| | | GAGATCGGCCCGCGCATGGCCGAGTTGAGCGG |
| | | TTCCCGGCTGGCCGCGCAGCAACAGATGGAAG |
| | | GCCTCCTGGCGCCGCACCGGCCCAAGGAGCCC |
| | | GCGTGGTTCCTGGCCACCGTCGGCGTCTCGCCC |
| | | GACCACCAGGGCAAGGGTCTGGGCAGCGCCGT |
| | | CGTGCTCCCCGGAGTGGAGGCGGCCGAGCGCG |
| | | CCGGGGTGCCCGCCTTCCTGGAGACCTCCGCGC |
| | | CCCGCAACCTCCCCTTCTACGAGCGGCTCGGCT |
| | | TCACCGTCACCGCCGACGTCGAGGTGCCCGAAG |
| | | GACCGCGCACCTGGTGCATGACCCGCAAGCCC |
| | | GGTGCC |
| SB07540 insert | 34 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| R34 (GS)3 | | FTFSNSGMSWVRQTPDKRLEWVASISDGGLYTHY |
| CD8 (H) | | PDSVKGRFTISRDNGKSTLYLQMSSLRSEDTAIYY |
| LIR1 (TM) | | CARQGVRPFFDYWGQGTTLTVSSGKPIPNPLLGL |
| LIR1 (ICD) | | DSTNGAALEIRAAFLEKENTALRTRAAELRKRVG |
| P2A PuroR | | RCRNIVSKYETRYGPLGSGSGSTTTPAPRPPTPAPT |
| | | IALQPLSLRPEACRPAAGGAVHTRGLDFACDVIGI |
| | | LVAVILLLLLLLLLFLILRHRRQGKHWTSTQRKAD |
| | | FQHPAGAVGPEPTDRGLQWRSSPAADAQEENLY |
| | | AAVKHTQPEDGVEMDTRSPHDEDPQAVTYAEVK |
| | | HSRPRREMASPPSPLSGEFLDTKDRQAEEDRQMD |
| | | TEAAASEAPQDVTYAQLHSLTLRREATEPPPSQEG |
| | | PSPAVPSIYATLAIHGSGATNFSLLKQAGDVEENP |
| | | GPMTEYKPTVRLATRDDVPRAVRTLAAAFADYP |
| | | ATRHTVDPDRHIERVTELQELFLTRVGLDIGKVW |
| | | VADDGAAVAVWTTPESVEAGAVFAEIGPRMAEL |
| | | SGSRLAAQQQMEGLLAPHRPKEPAWFLATVGVSP |
| | | DHQGKGLGSAVVLPGVEAAERAGVPAFLETSAPR |
| | | NLPFYERLGFTVTADVEVPEGPRTWCMTRKPGA |
| SB07541 insert | 35 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| E34-I (GS)3 | | TGGTATCAGCAGAAGCAGGGCAAGTCTCCTCAG |
| CD8 (H) | | CTGCTGGTGTTCAACGCCGAGACACTGCCTGAA |
| LIR1 (TM) | | GGCGTGCCCAGCAGATTTTCTGGAACAGGCAGC |
| LIR1 (ICD) | | GGCACCCACTTCAGCCTGAGAATCAATAGCCTG |
| P2A PuroR | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAATTACA |
| | | ATTAGAGCCGCATTCCTCGAGAAAGAAAACAC |
| | | CGCTCTGAGAACCGAGATTGCCGAACTCGAAA |
| | | AAGAAGTGGGGCGCTGCGAGAATATCGTGTCC |
| | | AAATACGAAACCCGCTACGGCCCACTCGGCTCT |
| | | GGTAGTGGATCTACCACGACGCCAGCGCCGCG |
| | | ACCACCAACACCGGCGCCCACCATCGCGTTGCA |
| | | GCCCCTGTCCCTGCGCCCAGAGGCGTGCCGGCC |
| | | AGCGGCGGGGGGCGCAGTGCACACGAGGGGGC |
| | | TGGACTTCGCCTGTGATGTTATAGGGATCCTGG |
| | | TGGCTGTCATACTCCTCTTGCTCCTCTTGTTGCT |
| | | GCTTTTTTTGATATTGCGCCACAGACGGCAGGG |
| | | AAAGCACTGGACTAGTACGCAGAGGAAAGCGG |
| | | ACTTCCAGCATCCCGCAGGAGCCGTGGGGCCTG |
| | | AACCCACTGATCGCGGCCTTCAATGGAGGTCTA |
| | | GCCCGGCGGCAGACGCACAAGAGGAAAACTTG |
| | | TACGCAGCCGTTAAGCACACCCAACCGGAGGA |
| | | CGGCGTTGAGATGGATACCCGCTCCCCTCACGA |
| | | TGAAGACCCTCAAGCAGTCACTTACGCGGAAGT |
| | | AAAGCATAGCCGCCCCAGACGGGAAATGGCTA |
| | | GCCCGCCGTCCCCCCTTAGCGGGGAATTTCTGG |
| | | ACACTAAAGATAGGCAGGCGGAAGAGGACCGC |
| | | CAAATGGATACAGAGGCGGCGGCAAGTGAAGC |
| | | ACCTCAAGACGTTACTTACGCTCAACTTCACAG |
| | | CCTTACCCTCAGGCGAGAAGCGACTGAACCACC |
| | | CCCTTCCCAAGAAGGGCCAAGCCCAGCGGTTCC |
| | | TTCTATCTATGCTACTCTTGCTATTCACGGAAGC |
| | | GGAGCTACTAACTTCAGCCTGCTGAAGCAGGCT |
| | | GGAGACGTGGAGGAGAACCCTGGACCTATGAC |
| | | CGAGTACAAGCCCACGGTGCGCCTCGCCACCCG |
| | | CGACGACGTCCCCAGGGCCGTACGCACCCTCGC |
| | | CGCCGCGTTCGCCGACTACCCCGCCACGCGCCA |
| | | CACCGTCGATCCGGACCGCCACATCGAGCGGGT |
| | | CACCGAGCTGCAAGAACTCTTCCTCACGCGCGT |
| | | CGGGCTCGACATCGGCAAGGTGTGGGTCGCGG |
| | | ACGACGGCGCCGCGGTGGCGGTCTGGACCACG |
| | | CCGGAGAGCGTCGAAGCGGGGGCGGTGTTCGC |
| | | CGAGATCGGCCCGCGCATGGCCGAGTTGAGCG |
| | | GTTCCCGGCTGGCCGCGCAGCAACAGATGGAA |
| | | GGCCTCCTGGCGCCGCACCGGCCCAAGGAGCC |
| | | CGCGTGGTTCCTGGCCACCGTCGGCGTCTCGCC |
| | | CGACCACCAGGGCAAGGGTCTGGGCAGCGCCG |
| | | TCGTGCTCCCCGGAGTGGAGGCGGCCGAGCGC |
| | | GCCGGGGTGCCCGCCTTCCTGGAGACCTCCGCG |
| | | CCCCGCAACCTCCCCTTCTACGAGCGGCTCGGC |
| | | TTCACCGTCACCGCCGACGTCGAGGTGCCCGAA |
| | | GGACCGCGCACCTGGTGCATGACCCGCAAGCC |
| | | CGGTGCC |
| SB07541 insert | 36 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| E34-I (GS)3 | | FTFSNSGMSWVRQTPDKRLEWVASISDGGLYTHY |
| CD8 (H) | | PDSVKGRFTISRDNGKSTLYLQMSSLRSEDTAIYY |
| LIR1 (TM) | | CARQGVRPFFDYWGQGTTLTVSSGKPIPNPLLGL |
| LIR1 (ICD) | | DSTNGAAITIRAAFLEKENTALRTEIAELEKEVGR |
| P2A PuroR | | CENIVSKYETRYGPLGSGSGSTTTPAPRPPTPAPTI |
| | | ALQPLSLRPEACRPAAGGAVHTRGLDFACDVIGIL |
| | | VAVILLLLLLLLLFLILRHRRQGKHWTSTQRKADF |
| | | QHPAGAVGPEPTDRGLQWRSSPAADAQEENLYA |
| | | AVKHTQPEDGVEMDTRSPHDEDPQAVTYAEVKH |
| | | SRPRREMASPPSPLSGEFLDTKDRQAEEDRQMDT |
| | | EAAASEAPQDVTYAQLHSLTLRREATEPPPSQEGP |
| | | SPAVPSIYATLAIHGSGATNFSLLKQAGDVEENPG |
| | | PMTEYKPTVRLATRDDVPRAVRTLAAAFADYPA |
| | | TRHTVDPDRHIERVTELQELFLTRVGLDIGKVWV |
| | | ADDGAAVAVWTTPESVEAGAVFAEIGPRMAELS |
| | | GSRLAAQQQMEGLLAPHRPKEPAWFLATVGVSP |
| | | DHQGKGLGSAVVLPGVEAAERAGVPAFLETSAPR |
| | | NLPFYERLGFTVTADVEVPEGPRTWCMTRKPGA |
| SB07542 insert | 37 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| E34-V (GS)3 | | TGGTATCAGCAGAAGCAGGGCAAGTCTCCTCAG |
| CD8 (H) | | CTGCTGGTGTTCAACGCCGAGACACTGCCTGAA |
| LIR1 (TM) | | GGCGTGCCCAGCAGATTTTCTGGAACAGGCAGC |
| LIR1 (ICD) | | GGCACCCACTTCAGCCTGAGAATCAATAGCCTG |
| P2A PuroR | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAATTACA |
| | | ATTAGAGCCGCCTTCCTGGAAAAAGAGAACAC |
| | | AGCCCTGAGAACCGAGGTGGCCGAACTCGAGA |
| | | AAGAAGTGGGCCGCTGCGAGAACATCGTGTCT |
| | | AAGTACGAGACAAGATACGGGCCCCTGGGCAG |
| | | CGGTAGCGGCTCCACCACGACGCCAGCGCCGC |
| | | GACCACCAACACCGGCGCCCACCATCGCGTTGC |
| | | AGCCCCTGTCCCTGCGCCCAGAGGCGTGCCGGC |
| | | CAGCGGCGGGGGGCGCAGTGCACACGAGGGGG |
| | | CTGGACTTCGCCTGTGATGTTATAGGGATCCTG |
| | | GTGGCTGTCATACTCCTCTTGCTCCTCTTGTTGC |
| | | TGCTTTTTTTGATATTGCGCCACAGACGGCAGG |
| | | GAAAGCACTGGACTAGTACGCAGAGGAAAGCG |
| | | GACTTCCAGCATCCCGCAGGAGCCGTGGGGCCT |
| | | GAACCCACTGATCGCGGCCTTCAATGGAGGTCT |
| | | AGCCCGGCGGCAGACGCACAAGAGGAAAACTT |
| | | GTACGCAGCCGTTAAGCACACCCAACCGGAGG |
| | | ACGGCGTTGAGATGGATACCCGCTCCCCTCACG |
| | | ATGAAGACCCTCAAGCAGTCACTTACGCGGAA |
| | | GTAAAGCATAGCCGCCCCAGACGGGAAATGGC |
| | | TAGCCCGCCGTCCCCCCTTAGCGGGGAATTTCT |
| | | GGACACTAAAGATAGGCAGGCGGAAGAGGACC |
| | | GCCAAATGGATACAGAGGCGGCGGCAAGTGAA |
| | | GCACCTCAAGACGTTACTTACGCTCAACTTCAC |
| | | AGCCTTACCCTCAGGCGAGAAGCGACTGAACC |
| | | ACCCCCTTCCCAAGAAGGGCCAAGCCCAGCGG |
| | | TTCCTTCTATCTATGCTACTCTTGCTATTCACGG |
| | | AAGCGGAGCTACTAACTTCAGCCTGCTGAAGCA |
| | | GGCTGGAGACGTGGAGGAGAACCCTGGACCTA |
| | | TGACCGAGTACAAGCCCACGGTGCGCCTCGCCA |
| | | CCCGCGACGACGTCCCCAGGGCCGTACGCACCC |
| | | TCGCCGCCGCGTTCGCCGACTACCCCGCCACGC |
| | | GCCACACCGTCGATCCGGACCGCCACATCGAGC |
| | | GGGTCACCGAGCTGCAAGAACTCTTCCTCACGC |
| | | GCGTCGGGCTCGACATCGGCAAGGTGTGGGTCG |
| | | CGGACGACGGCGCCGCGGTGGCGGTCTGGACC |
| | | ACGCCGGAGAGCGTCGAAGCGGGGGCGGTGTT |
| | | CGCCGAGATCGGCCCGCGCATGGCCGAGTTGA |
| | | GCGGTTCCCGGCTGGCCGCGCAGCAACAGATG |
| | | GAAGGCCTCCTGGCGCCGCACCGGCCCAAGGA |
| | | GCCCGCGTGGTTCCTGGCCACCGTCGGCGTCTC |
| | | GCCCGACCACCAGGGCAAGGGTCTGGGCAGCG |
| | | CCGTCGTGCTCCCCGGAGTGGAGGCGGCCGAGC |
| | | GCGCCGGGGTGCCCGCCTTCCTGGAGACCTCCG |
| | | CGCCCCGCAACCTCCCCTTCTACGAGCGGCTCG |
| | | GCTTCACCGTCACCGCCGACGTCGAGGTGCCCG |
| | | AAGGACCGCGCACCTGGTGCATGACCCGCAAG |
| | | CCCGGTGCC |
| SB07542 insert | 38 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| E34-V (GS)3 | | FTFSNSGMSWVRQTPDKRLEWVASISDGGLYTHY |
| CD8 (H) | | PDSVKGRFTISRDNGKSTLYLQMSSLRSEDTAIYY |
| LIR1 (TM) | | CARQGVRPFFDYWGQGTTLTVSSGKPIPNPLLGL |
| LIR1 (ICD) | | DSTNGAAITIRAAFLEKENTALRTEVAELEKEVGR |
| P2A PuroR | | CENIVSKYETRYGPLGSGSGSTTTPAPRPPTPAPTI |
| | | ALQPLSLRPEACRPAAGGAVHTRGLDFACDVIGIL |
| | | VAVILLLLLLLLLFLILRHRRQGKHWTSTQRKADF |
| | | QHPAGAVGPEPTDRGLQWRSSPAADAQEENLYA |
| | | AVKHTQPEDGVEMDTRSPHDEDPQAVTYAEVKH |
| | | SRPRREMASPPSPLSGEFLDTKDRQAEEDRQMDT |
| | | EAAASEAPQDVTYAQLHSLTLRREATEPPPSQEGP |
| | | SPAVPSIYATLAIHGSGATNFSLLKQAGDVEENPG |
| | | PMTEYKPTVRLATRDDVPRAVRTLAAAFADYPA |
| | | TRHTVDPDRHIERVTELQELFLTRVGLDIGKVWV |
| | | ADDGAAVAVWTTPESVEAGAVFAEIGPRMAELS |
| | | GSRLAAQQQMEGLLAPHRPKEPAWFLATVGVSP |
| | | DHQGKGLGSAVVLPGVEAAERAGVPAFLETSAPR |
| | | NLPFYERLGFTVTADVEVPEGPRTWCMTRKPGA |
| SB07543 insert | 39 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| E34-N (GS)3 | | TGGTATCAGCAGAAGCAGGGCAAGTCTCCTCAG |
| CD8 (H) | | CTGCTGGTGTTCAACGCCGAGACACTGCCTGAA |
| LIR1 (TM) | | GGCGTGCCCAGCAGATTTTCTGGAACAGGCAGC |
| LIR1 (ICD) | | GGCACCCACTTCAGCCTGAGAATCAATAGCCTG |
| P2A PuroR | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAATTACA |
| | | ATCAGAGCCGCCTTCCTCGAGAAAGAGAACAC |
| | | AGCCCTGAGAACCGAGAACGCCGAGCTGGAAA |
| | | AAGAAGTGGGCCGCTGCGAGAACATCGTGTCT |
| | | AAGTACGAGACAAGATACGGCCCACTCGGCTC |
| | | CGGATCTGGAAGCACCACGACGCCAGCGCCGC |
| | | GACCACCAACACCGGCGCCCACCATCGCGTTGC |
| | | AGCCCCTGTCCCTGCGCCCAGAGGCGTGCCGGC |
| | | CAGCGGGGGGGGCGCAGTGCACACGAGGGGG |
| | | CTGGACTTCGCCTGTGATGTTATAGGGATCCTG |
| | | GTGGCTGTCATACTCCTCTTGCTCCTCTTGTTGC |
| | | TGCTTTTTTTGATATTGCGCCACAGACGGCAGG |
| | | GAAAGCACTGGACTAGTACGCAGAGGAAAGCG |
| | | GACTTCCAGCATCCCGCAGGAGCCGTGGGGCCT |
| | | GAACCCACTGATCGCGGCCTTCAATGGAGGTCT |
| | | AGCCCGGCGGCAGACGCACAAGAGGAAAACTT |
| | | GTACGCAGCCGTTAAGCACACCCAACCGGAGG |
| | | ACGGCGTTGAGATGGATACCCGCTCCCCTCACG |
| | | ATGAAGACCCTCAAGCAGTCACTTACGCGGAA |
| | | GTAAAGCATAGCCGCCCCAGACGGGAAATGGC |
| | | TAGCCCGCCGTCCCCCCTTAGCGGGGAATTTCT |
| | | GGACACTAAAGATAGGCAGGCGGAAGAGGACC |
| | | GCCAAATGGATACAGAGGCGGCGGCAAGTGAA |
| | | GCACCTCAAGACGTTACTTACGCTCAACTTCAC |
| | | AGCCTTACCCTCAGGCGAGAAGCGACTGAACC |
| | | ACCCCCTTCCCAAGAAGGGCCAAGCCCAGCGG |
| | | TTCCTTCTATCTATGCTACTCTTGCTATTCACGG |
| | | AAGCGGAGCTACTAACTTCAGCCTGCTGAAGCA |
| | | GGCTGGAGACGTGGAGGAGAACCCTGGACCTA |
| | | TGACCGAGTACAAGCCCACGGTGCGCCTCGCCA |
| | | CCCGCGACGACGTCCCCAGGGCCGTACGCACCC |
| | | TCGCCGCCGCGTTCGCCGACTACCCCGCCACGC |
| | | GCCACACCGTCGATCCGGACCGCCACATCGAGC |
| | | GGGTCACCGAGCTGCAAGAACTCTTCCTCACGC |
| | | GCGTCGGGCTCGACATCGGCAAGGTGTGGGTCG |
| | | CGGACGACGGCGCCGCGGTGGCGGTCTGGACC |
| | | ACGCCGGAGAGCGTCGAAGCGGGGGCGGTGTT |
| | | CGCCGAGATCGGCCCGCGCATGGCCGAGTTGA |
| | | GCGGTTCCCGGCTGGCCGCGCAGCAACAGATG |
| | | GAAGGCCTCCTGGCGCCGCACCGGCCCAAGGA |
| | | GCCCGCGTGGTTCCTGGCCACCGTCGGCGTCTC |
| | | GCCCGACCACCAGGGCAAGGGTCTGGGCAGCG |
| | | CCGTCGTGCTCCCCGGAGTGGAGGCGGCCGAGC |
| | | GCGCCGGGGTGCCCGCCTTCCTGGAGACCTCCG |
| | | CGCCCCGCAACCTCCCCTTCTACGAGCGGCTCG |
| | | GCTTCACCGTCACCGCCGACGTCGAGGTGCCCG |
| | | AAGGACCGCGCACCTGGTGCATGACCCGCAAG |
| | | CCCGGTGCC |
| SB07543 insert | 40 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| E34-N (GS)3 | | FTFSNSGMSWVRQTPDKRLEWVASISDGGLYTHY |
| CD8 (H) | | PDSVKGRFTISRDNGKSTLYLQMSSLRSEDTAIYY |
| LIR1 (TM) | | CARQGVRPFFDYWGQGTTLTVSSGKPIPNPLLGL |
| LIR1 (ICD) | | DSTNGAAITIRAAFLEKENTALRTENAELEKEVGR |
| P2A PuroR | | CENIVSKYETRYGPLGSGSGSTTTPAPRPPTPAPTI |
| | | ALQPLSLRPEACRPAAGGAVHTRGLDFACDVIGIL |
| | | VAVILLLLLLLLLFLILRHRRQGKHWTSTQRKADF |
| | | QHPAGAVGPEPTDRGLQWRSSPAADAQEENLYA |
| | | AVKHTQPEDGVEMDTRSPHDEDPQAVTYAEVKH |
| | | SRPRREMASPPSPLSGEFLDTKDRQAEEDRQMDT |
| | | EAAASEAPQDVTYAQLHSLTLRREATEPPPSQEGP |
| | | SPAVPSIYATLAIHGSGATNFSLLKQAGDVEENPG |
| | | PMTEYKPTVRLATRDDVPRAVRTLAAAFADYPA |
| | | TRHTVDPDRHIERVTELQELFLTRVGLDIGKVWV |
| | | ADDGAAVAVWTTPESVEAGAVFAEIGPRMAELS |
| | | GSRLAAQQQMEGLLAPHRPKEPAWFLATVGVSP |
| | | DHQGKGLGSAVVLPGVEAAERAGVPAFLETSAPR |
| | | NLPFYERLGFTVTADVEVPEGPRTWCMTRKPGA |
| SB07544 insert | 41 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| CD8 (H) | | ATGGTATCAGCAGAAGCCTGGCAAGGCCCCTA |
| CD28 (TM) | | AGCTGCTGATCTACTGGACCAGCACCAGACACA |
| CD28 (ICD) z | | CAGGCGTGCCCAGCAGATTTTCTGGCAGCGGCT |
| (GS)3 R34 | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAACCACGACGCCAGCGCCGCGACCACC |
| | | AACACCGGCGCCCACCATCGCGTTGCAGCCCCT |
| | | GTCCCTGCGCCCAGAGGCGTGCCGGCCAGCGG |
| | | CGGGGGGCGCAGTGCACACGAGGGGGCTGGAC |
| | | TTCGCCTGTGATTTTTGGGTGCTGGTGGTGGTTG |
| | | GTGGAGTCCTGGCTTGCTATAGCTTGCTAGTAA |
| | | CAGTGGCCTTTATTATTTTCTGGGTGAGGAGTA |
| | | AGAGGAGCAGGCTCCTGCACAGTGACTACATG |
| | | AACATGACTCCCCGCCGCCCCGGGCCCACCCGC |
| | | AAGCATTACCAGCCCTATGCCCCACCACGCGAC |
| | | TTCGCAGCCTATCGCTCCAGAGTGAAGTTCAGC |
| | | AGGAGCGCAGACGCCCCCGCGTACAAGCAGGG |
| | | CCAGAACCAGCTCTATAACGAGCTCAATCTAGG |
| | | ACGAAGAGAGGAGTACGATGTTTTGGACAAGA |
| | | GACGTGGCCGGGACCCTGAGATGGGGGGAAAG |
| | | CCGAGAAGGAAGAACCCTCAGGAAGGCCTGTA |
| | | CAATGAACTGCAGAAAGATAAGATGGCGGAGG |
| | | CCTACAGTGAGATTGGGATGAAAGGCGAGCGC |
| | | CGGAGGGGCAAGGGGCACGATGGCCTTTACCA |
| | | GGGTCTCAGTACAGCCACCAAGGACACCTACG |
| | | ACGCCCTTCACATGCAGGCCCTGCCCCCTCGCG |
| | | GATCAGGATCTGGCAGCCTGGAAATCAGGGCC |
| | | GCCTTCCTGGAAAAAGAGAACACAGCCCTGCG |
| | | GACAAGAGCCGCCGAGCTGAGAAAACGCGTGG |
| | | GCAGATGCCGGAACATCGTGTCTAAGTACGAG |
| | | ACAAGATACGGCCCTCTG |
| SB07544 insert | 42 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| CD8 (H) | | WMSWVRQAPGKGLEWIGEIHPDSSTINYAPSLKD |
| CD28 (TM) | | RFTISRDNAKNTLFLQMDSLRPEDTGVYFCASLYF |
| CD28 (ICD) z | | GFPWFAYWGQGTPVTVSSEQKLISEEDLNGAATT |
| (GS)3 R34 | | TPAPRPPTPAPTIALQPLSLRPEACRPAAGGAVHT |
| | | RGLDFACDFWVLVVVGGVLACYSLLVTVAFIIFW |
| | | VRSKRSRLLHSDYMNMTPRRPGPTRKHYQPYAPP |
| | | RDFAAYRSRVKFSRSADAPAYKQGQNQLYNELN |
| | | LGRREEYDVLDKRRGRDPEMGGKPRRKNPQEGL |
| | | YNELQKDKMAEAYSEIGMKGERRRGKGHDGLY |
| | | QGLSTATKDTYDALHMQALPPRGSGSGSLEIRAA |
| | | FLEKENTALRTRAAELRKRVGRCRNIVSKYETRY |
| | | GPL |
| SB07545 insert | 43 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| CD8 (H) | | ATGGTATCAGCAGAAGCCTGGCAAGGCCCCTA |
| CD28 (TM) | | AGCTGCTGATCTACTGGACCAGCACCAGACACA |
| CD28 (ICD) z | | CAGGCGTGCCCAGCAGATTTTCTGGCAGCGGCT |
| (GS)3 E34-I | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAACCACGACGCCAGCGCCGCGACCACC |
| | | AACACCGGCGCCCACCATCGCGTTGCAGCCCCT |
| | | GTCCCTGCGCCCAGAGGCGTGCCGGCCAGCGG |
| | | CGGGGGGCGCAGTGCACACGAGGGGGCTGGAC |
| | | TTCGCCTGTGATTTTTGGGTGCTGGTGGTGGTTG |
| | | GTGGAGTCCTGGCTTGCTATAGCTTGCTAGTAA |
| | | CAGTGGCCTTTATTATTTTCTGGGTGAGGAGTA |
| | | AGAGGAGCAGGCTCCTGCACAGTGACTACATG |
| | | AACATGACTCCCCGCCGCCCCGGGCCCACCCGC |
| | | AAGCATTACCAGCCCTATGCCCCACCACGCGAC |
| | | TTCGCAGCCTATCGCTCCAGAGTGAAGTTCAGC |
| | | AGGAGCGCAGACGCCCCCGCGTACAAGCAGGG |
| | | CCAGAACCAGCTCTATAACGAGCTCAATCTAGG |
| | | ACGAAGAGAGGAGTACGATGTTTTGGACAAGA |
| | | GACGTGGCCGGGACCCTGAGATGGGGGGAAAG |
| | | CCGAGAAGGAAGAACCCTCAGGAAGGCCTGTA |
| | | CAATGAACTGCAGAAAGATAAGATGGCGGAGG |
| | | CCTACAGTGAGATTGGGATGAAAGGCGAGCGC |
| | | CGGAGGGGCAAGGGGCACGATGGCCTTTACCA |
| | | GGGTCTCAGTACAGCCACCAAGGACACCTACG |
| | | ACGCCCTTCACATGCAGGCCCTGCCCCCTCGCG |
| | | GAAGCGGATCCGGCAGCATTACAATTAGAGCC |
| | | GCATTCCTCGAGAAAGAAAACACCGCTCTGAG |
| | | AACCGAGATTGCCGAACTCGAAAAAGAAGTGG |
| | | GGCGCTGCGAGAATATCGTGTCCAAATACGAA |
| | | ACCCGCTACGGCCCACTC |
| SB07545 insert | 44 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| CD8 (H) | | WMSWVRQAPGKGLEWIGEIHPDSSTINYAPSLKD |
| CD28 (TM) | | RFTISRDNAKNTLFLQMDSLRPEDTGVYFCASLYF |
| CD28 (ICD) z | | GFPWFAYWGQGTPVTVSSEQKLISEEDLNGAATT |
| (GS)3 E34-I | | TPAPRPPTPAPTIALQPLSLRPEACRPAAGGAVHT |
| | | RGLDFACDFWVLVVVGGVLACYSLLVTVAFIIFW |
| | | VRSKRSRLLHSDYMNMTPRRPGPTRKHYQPYAPP |
| | | RDFAAYRSRVKFSRSADAPAYKQGQNQLYNELN |
| | | LGRREEYDVLDKRRGRDPEMGGKPRRKNPQEGL |
| | | YNELQKDKMAEAYSEIGMKGERRRGKGHDGLY |
| | | QGLSTATKDTYDALHMQALPPRGSGSGSITIRAAF |
| | | LEKENTALRTEIAELEKEVGRCENIVSKYETRYGP |
| | | L |
| SB07546 insert | 45 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| CD8 (H) | | ATGGTATCAGCAGAAGCCTGGCAAGGCCCCTA |
| CD28 (TM) | | AGCTGCTGATCTACTGGACCAGCACCAGACACA |
| CD28 (ICD) z | | CAGGCGTGCCCAGCAGATTTTCTGGCAGCGGCT |
| (GS)3 E34-V | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAACCACGACGCCAGCGCCGCGACCACC |
| | | AACACCGGCGCCCACCATCGCGTTGCAGCCCCT |
| | | GTCCCTGCGCCCAGAGGCGTGCCGGCCAGCGG |
| | | CGGGGGGCGCAGTGCACACGAGGGGGCTGGAC |
| | | TTCGCCTGTGATTTTTGGGTGCTGGTGGTGGTTG |
| | | GTGGAGTCCTGGCTTGCTATAGCTTGCTAGTAA |
| | | CAGTGGCCTTTATTATTTTCTGGGTGAGGAGTA |
| | | AGAGGAGCAGGCTCCTGCACAGTGACTACATG |
| | | AACATGACTCCCCGCCGCCCCGGGCCCACCCGC |
| | | AAGCATTACCAGCCCTATGCCCCACCACGCGAC |
| | | TTCGCAGCCTATCGCTCCAGAGTGAAGTTCAGC |
| | | AGGAGCGCAGACGCCCCCGCGTACAAGCAGGG |
| | | CCAGAACCAGCTCTATAACGAGCTCAATCTAGG |
| | | ACGAAGAGAGGAGTACGATGTTTTGGACAAGA |
| | | GACGTGGCCGGGACCCTGAGATGGGGGGAAAG |
| | | CCGAGAAGGAAGAACCCTCAGGAAGGCCTGTA |
| | | CAATGAACTGCAGAAAGATAAGATGGCGGAGG |
| | | CCTACAGTGAGATTGGGATGAAAGGCGAGCGC |
| | | CGGAGGGGCAAGGGGCACGATGGCCTTTACCA |
| | | GGGTCTCAGTACAGCCACCAAGGACACCTACG |
| | | ACGCCCTTCACATGCAGGCCCTGCCCCCTCGCG |
| | | GTAGCGGCTCCGGCTCCATTACAATTAGAGCCG |
| | | CCTTCCTGGAAAAAGAGAACACAGCCCTGAGA |
| | | ACCGAGGTGGCCGAACTCGAGAAAGAAGTGGG |
| | | CCGCTGCGAGAACATCGTGTCTAAGTACGAGAC |
| | | AAGATACGGGCCCCTG |
| SB07546 insert | 46 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| CD8 (H) | | WMSWVRQAPGKGLEWIGEIHPDSSTINYAPSLKD |
| CD28 (TM) | | RFTISRDNAKNTLFLQMDSLRPEDTGVYFCASLYF |
| CD28 (ICD) z | | GFPWFAYWGQGTPVTVSSEQKLISEEDLNGAATT |
| (GS)3 E34-V | | TPAPRPPTPAPTIALQPLSLRPEACRPAAGGAVHT |
| | | RGLDFACDFWVLVVVGGVLACYSLLVTVAFIIFW |
| | | VRSKRSRLLHSDYMNMTPRRPGPTRKHYQPYAPP |
| | | RDFAAYRSRVKFSRSADAPAYKQGQNQLYNELN |
| | | LGRREEYDVLDKRRGRDPEMGGKPRRKNPQEGL |
| | | YNELQKDKMAEAYSEIGMKGERRRGKGHDGLY |
| | | QGLSTATKDTYDALHMQALPPRGSGSGSITIRAAF |
| | | LEKENTALRTEVAELEKEVGRCENIVSKYETRYG |
| | | PL |
| SB07547 insert | 47 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc CD8 (H) | | AAGGCCTCTCAGGACGTGGGCACAAGCGTGGC |
| CD28 (TM) | | ATGGTATCAGCAGAAGCCTGGCAAGGCCCCTA |
| CD28 (ICD) z | | AGCTGCTGATCTACTGGACCAGCACCAGACACA |
| (GS)3 E34-N | | CAGGCGTGCCCAGCAGATTTTCTGGCAGCGGCT |
| | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAACCACGACGCCAGCGCCGCGACCACC |
| | | AACACCGGCGCCCACCATCGCGTTGCAGCCCCT |
| | | GTCCCTGCGCCCAGAGGCGTGCCGGCCAGCGG |
| | | CGGGGGGCGCAGTGCACACGAGGGGGCTGGAC |
| | | TTCGCCTGTGATTTTTGGGTGCTGGTGGTGGTTG |
| | | GTGGAGTCCTGGCTTGCTATAGCTTGCTAGTAA |
| | | CAGTGGCCTTTATTATTTTCTGGGTGAGGAGTA |
| | | AGAGGAGCAGGCTCCTGCACAGTGACTACATG |
| | | AACATGACTCCCCGCCGCCCCGGGCCCACCCGC |
| | | AAGCATTACCAGCCCTATGCCCCACCACGCGAC |
| | | TTCGCAGCCTATCGCTCCAGAGTGAAGTTCAGC |
| | | AGGAGCGCAGACGCCCCCGCGTACAAGCAGGG |
| | | CCAGAACCAGCTCTATAACGAGCTCAATCTAGG |
| | | ACGAAGAGAGGAGTACGATGTTTTGGACAAGA |
| | | GACGTGGCCGGGACCCTGAGATGGGGGGAAAG |
| | | CCGAGAAGGAAGAACCCTCAGGAAGGCCTGTA |
| | | CAATGAACTGCAGAAAGATAAGATGGCGGAGG |
| | | CCTACAGTGAGATTGGGATGAAAGGCGAGCGC |
| | | CGGAGGGGCAAGGGGCACGATGGCCTTTACCA |
| | | GGGTCTCAGTACAGCCACCAAGGACACCTACG |
| | | ACGCCCTTCACATGCAGGCCCTGCCCCCTCGCG |
| | | GATCTGGCTCTGGATCCATTACAATCAGAGCCG |
| | | CCTTCCTCGAGAAAGAGAACACAGCCCTGAGA |
| | | ACCGAGAACGCCGAGCTGGAAAAAGAAGTGGG |
| | | CCGCTGCGAGAACATCGTGTCTAAGTACGAGAC |
| | | AAGATACGGCCCACTC |
| SB07547 insert | 48 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc CD8 (H) | | SGSEVQLVESGGGVVQPGRSLRLSCSASGFDFTTY |
| CD28 (TM) | | WMSWVRQAPGKGLEWIGEIHPDSSTINYAPSLKD |
| CD28 (ICD) z | | RFTISRDNAKNTLFLQMDSLRPEDTGVYFCASLYF |
| (GS)3 E34-N | | GFPWFAYWGQGTPVTVSSEQKLISEEDLNGAATT |
| | | TPAPRPPTPAPTIALQPLSLRPEACRPAAGGAVHT |
| | | RGLDFACDFWVLVVVGGVLACYSLLVTVAFIIFW |
| | | VRSKRSRLLHSDYMNMTPRRPGPTRKHYQPYAPP |
| | | RDFAAYRSRVKFSRSADAPAYKQGQNQLYNELN |
| | | LGRREEYDVLDKRRGRDPEMGGKPRRKNPQEGL |
| | | YNELQKDKMAEAYSEIGMKGERRRGKGHDGLY |
| | | QGLSTATKDTYDALHMQALPPRGSGSGSITIRAAF |
| | | LEKENTALRTENAELEKEVGRCENIVSKYETRYG |
| | | PL |
| SB07548 insert | 49 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| CD8 (H) | | TGGTATCAGCAGAAGCAGGGCAAGTCTCCTCAG |
| LIR1 (TM) | | CTGCTGGTGTTCAACGCCGAGACACTGCCTGAA |
| LIR1 (ICD) | | GGCGTGCCCAGCAGATTTTCTGGAACAGGCAGC |
| (GS)3 R34 | | GGCACCCACTTCAGCCTGAGAATCAATAGCCTG |
| P2A PuroR | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAACCACG |
| | | ACGCCAGCGCCGCGACCACCAACACCGGCGCC |
| | | CACCATCGCGTTGCAGCCCCTGTCCCTGCGCCC |
| | | AGAGGCGTGCCGGCCAGCGGCGGGGGGCGCAG |
| | | TGCACACGAGGGGGCTGGACTTCGCCTGTGATG |
| | | TTATAGGGATCCTGGTGGCTGTCATACTCCTCTT |
| | | GCTCCTCTTGTTGCTGCTTTTTTTGATATTGCGC |
| | | CACAGACGGCAGGGAAAGCACTGGACTAGTAC |
| | | GCAGAGGAAAGCGGACTTCCAGCATCCCGCAG |
| | | GAGCCGTGGGGCCTGAACCCACTGATCGCGGCC |
| | | TTCAATGGAGGTCTAGCCCGGCGGCAGACGCAC |
| | | AAGAGGAAAACTTGTACGCAGCCGTTAAGCAC |
| | | ACCCAACCGGAGGACGGCGTTGAGATGGATAC |
| | | CCGCTCCCCTCACGATGAAGACCCTCAAGCAGT |
| | | CACTTACGCGGAAGTAAAGCATAGCCGCCCCA |
| | | GACGGGAAATGGCTAGCCCGCCGTCCCCCCTTA |
| | | GCGGGGAATTTCTGGACACTAAAGATAGGCAG |
| | | GCGGAAGAGGACCGCCAAATGGATACAGAGGC |
| | | GGCGGCAAGTGAAGCACCTCAAGACGTTACTTA |
| | | CGCTCAACTTCACAGCCTTACCCTCAGGCGAGA |
| | | AGCGACTGAACCACCCCCTTCCCAAGAAGGGC |
| | | CAAGCCCAGCGGTTCCTTCTATCTATGCTACTCT |
| | | TGCTATTCACGGATCAGGATCTGGCAGCCTGGA |
| | | AATCAGGGCCGCCTTCCTGGAAAAAGAGAACA |
| | | CAGCCCTGCGGACAAGAGCCGCCGAGCTGAGA |
| | | AAACGCGTGGGCAGATGCCGGAACATCGTGTCT |
| | | AAGTACGAGACAAGATACGGCCCTCTGGGAAG |
| | | CGGAGCTACTAACTTCAGCCTGCTGAAGCAGGC |
| | | TGGAGACGTGGAGGAGAACCCTGGACCTATGA |
| | | CCGAGTACAAGCCCACGGTGCGCCTCGCCACCC |
| | | GCGACGACGTCCCCAGGGCCGTACGCACCCTCG |
| | | CCGCCGCGTTCGCCGACTACCCCGCCACGCGCC |
| | | ACACCGTCGATCCGGACCGCCACATCGAGCGG |
| | | GTCACCGAGCTGCAAGAACTCTTCCTCACGCGC |
| | | GTCGGGCTCGACATCGGCAAGGTGTGGGTCGCG |
| | | GACGACGGCGCCGCGGTGGCGGTCTGGACCAC |
| | | GCCGGAGAGCGTCGAAGCGGGGGCGGTGTTCG |
| | | CCGAGATCGGCCCGCGCATGGCCGAGTTGAGC |
| | | GGTTCCCGGCTGGCCGCGCAGCAACAGATGGA |
| | | AGGCCTCCTGGCGCCGCACCGGCCCAAGGAGC |
| | | CCGCGTGGTTCCTGGCCACCGTCGGCGTCTCGC |
| | | CCGACCACCAGGGCAAGGGTCTGGGCAGCGCC |
| | | GTCGTGCTCCCCGGAGTGGAGGCGGCCGAGCG |
| | | CGCCGGGGTGCCCGCCTTCCTGGAGACCTCCGC |
| | | GCCCCGCAACCTCCCCTTCTACGAGCGGCTCGG |
| | | CTTCACCGTCACCGCCGACGTCGAGGTGCCCGA |
| | | AGGACCGCGCACCTGGTGCATGACCCGCAAGC |
| | | CCGGTGCC |
| SB07548 insert | 50 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| CD8 (H) | | FTFSNSGMSWVRQTPDKRLEWVASISDGGLYTHY |
| LIR1 (TM) | | PDSVKGRFTISRDNGKSTLYLQMSSLRSEDTAIYY |
| LIR1 (ICD) | | CARQGVRPFFDYWGQGTTLTVSSGKPIPNPLLGL |
| (GS)3 R34 | | DSTNGAATTTPAPRPPTPAPTIALQPLSLRPEACRP |
| P2A PuroR | | AAGGAVHTRGLDFACDVIGILVAVILLLLLLLLLF |
| | | LILRHRRQGKHWTSTQRKADFQHPAGAVGPEPTD |
| | | RGLQWRSSPAADAQEENLYAAVKHTQPEDGVEM |
| | | DTRSPHDEDPQAVTYAEVKHSRPRREMASPPSPL |
| | | SGEFLDTKDRQAEEDRQMDTEAAASEAPQDVTY |
| | | AQLHSLTLRREATEPPPSQEGPSPAVPSIYATLAIH |
| | | GSGSGSLEIRAAFLEKENTALRTRAAELRKRVGRC |
| | | RNIVSKYETRYGPLGSGATNFSLLKQAGDVEENP |
| | | GPMTEYKPTVRLATRDDVPRAVRTLAAAFADYP |
| | | ATRHTVDPDRHIERVTELQELFLTRVGLDIGKVW |
| | | VADDGAAVAVWTTPESVEAGAVFAEIGPRMAEL |
| | | SGSRLAAQQQMEGLLAPHRPKEPAWFLATVGVSP |
| | | DHQGKGLGSAVVLPGVEAAERAGVPAFLETSAPR |
| | | NLPFYERLGFTVTADVEVPEGPRTWCMTRKPGA |
| SB07549 insert | 51 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| CD8 (H) | | TGGTATCAGCAGAAGCAGGGCAAGTCTCCTCAG |
| LIR1 (TM) | | CTGCTGGTGTTCAACGCCGAGACACTGCCTGAA |
| LIR1 (ICD) | | GGCGTGCCCAGCAGATTTTCTGGAACAGGCAGC |
| (GS)3 E34-I | | GGCACCCACTTCAGCCTGAGAATCAATAGCCTG |
| P2A PuroR | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAACCACG |
| | | ACGCCAGCGCCGCGACCACCAACACCGGCGCC |
| | | CACCATCGCGTTGCAGCCCCTGTCCCTGCGCCC |
| | | AGAGGCGTGCCGGCCAGCGGCGGGGGGCGCAG |
| | | TGCACACGAGGGGGCTGGACTTCGCCTGTGATG |
| | | TTATAGGGATCCTGGTGGCTGTCATACTCCTCTT |
| | | GCTCCTCTTGTTGCTGCTTTTTTTGATATTGCGC |
| | | CACAGACGGCAGGGAAAGCACTGGACTAGTAC |
| | | GCAGAGGAAAGCGGACTTCCAGCATCCCGCAG |
| | | GAGCCGTGGGGCCTGAACCCACTGATCGCGGCC |
| | | TTCAATGGAGGTCTAGCCCGGCGGCAGACGCAC |
| | | AAGAGGAAAACTTGTACGCAGCCGTTAAGCAC |
| | | ACCCAACCGGAGGACGGCGTTGAGATGGATAC |
| | | CCGCTCCCCTCACGATGAAGACCCTCAAGCAGT |
| | | CACTTACGCGGAAGTAAAGCATAGCCGCCCCA |
| | | GACGGGAAATGGCTAGCCCGCCGTCCCCCCTTA |
| | | GCGGGGAATTTCTGGACACTAAAGATAGGCAG |
| | | GCGGAAGAGGACCGCCAAATGGATACAGAGGC |
| | | GGCGGCAAGTGAAGCACCTCAAGACGTTACTTA |
| | | CGCTCAACTTCACAGCCTTACCCTCAGGCGAGA |
| | | AGCGACTGAACCACCCCCTTCCCAAGAAGGGC |
| | | CAAGCCCAGCGGTTCCTTCTATCTATGCTACTCT |
| | | TGCTATTCACGGAAGCGGATCCGGCAGCATTAC |
| | | AATTAGAGCCGCATTCCTCGAGAAAGAAAACA |
| | | CCGCTCTGAGAACCGAGATTGCCGAACTCGAAA |
| | | AAGAAGTGGGGCGCTGCGAGAATATCGTGTCC |
| | | AAATACGAAACCCGCTACGGCCCACTCGGAAG |
| | | CGGAGCTACTAACTTCAGCCTGCTGAAGCAGGC |
| | | TGGAGACGTGGAGGAGAACCCTGGACCTATGA |
| | | CCGAGTACAAGCCCACGGTGCGCCTCGCCACCC |
| | | GCGACGACGTCCCCAGGGCCGTACGCACCCTCG |
| | | CCGCCGCGTTCGCCGACTACCCCGCCACGCGCC |
| | | ACACCGTCGATCCGGACCGCCACATCGAGCGG |
| | | GTCACCGAGCTGCAAGAACTCTTCCTCACGCGC |
| | | GTCGGGCTCGACATCGGCAAGGTGTGGGTCGCG |
| | | GACGACGGCGCCGCGGTGGCGGTCTGGACCAC |
| | | GCCGGAGAGCGTCGAAGCGGGGGCGGTGTTCG |
| | | CCGAGATCGGCCCGCGCATGGCCGAGTTGAGC |
| | | GGTTCCCGGCTGGCCGCGCAGCAACAGATGGA |
| | | AGGCCTCCTGGCGCCGCACCGGCCCAAGGAGC |
| | | CCGCGTGGTTCCTGGCCACCGTCGGCGTCTCGC |
| | | CCGACCACCAGGGCAAGGGTCTGGGCAGCGCC |
| | | GTCGTGCTCCCCGGAGTGGAGGCGGCCGAGCG |
| | | CGCCGGGGTGCCCGCCTTCCTGGAGACCTCCGC |
| | | GCCCCGCAACCTCCCCTTCTACGAGCGGCTCGG |
| | | CTTCACCGTCACCGCCGACGTCGAGGTGCCCGA |
| | | AGGACCGCGCACCTGGTGCATGACCCGCAAGC |
| | | CCGGTGCC |
| SB07549 insert | 52 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| CD8 (H) | | FTFSNSGMSWVRQTPDKRLEWVASISDGGLYTHY |
| LIR1 (TM) | | PDSVKGRFTISRDNGKSTLYLQMSSLRSEDTAIYY |
| LIR1 (ICD) | | CARQGVRPFFDYWGQGTTLTVSSGKPIPNPLLGL |
| (GS)3 E34-I | | DSTNGAATTTPAPRPPTPAPTIALQPLSLRPEACRP |
| P2A PuroR | | AAGGAVHTRGLDFACDVIGILVAVILLLLLLLLLF |
| | | LILRHRRQGKHWTSTQRKADFQHPAGAVGPEPTD |
| | | RGLQWRSSPAADAQEENLYAAVKHTQPEDGVEM |
| | | DTRSPHDEDPQAVTYAEVKHSRPRREMASPPSPL |
| | | SGEFLDTKDRQAEEDRQMDTEAAASEAPQDVTY |
| | | AQLHSLTLRREATEPPPSQEGPSPAVPSIYATLAIH |
| | | GSGSGSITIRAAFLEKENTALRTEIAELEKEVGRCE |
| | | NIVSKYETRYGPLGSGATNFSLLKQAGDVEENPG |
| | | PMTEYKPTVRLATRDDVPRAVRTLAAAFADYPA |
| | | TRHTVDPDRHIERVTELQELFLTRVGLDIGKVWV |
| | | ADDGAAVAVWTTPESVEAGAVFAEIGPRMAELS |
| | | GSRLAAQQQMEGLLAPHRPKEPAWFLATVGVSP |
| | | DHQGKGLGSAVVLPGVEAAERAGVPAFLETSAPR |
| | | NLPFYERLGFTVTADVEVPEGPRTWCMTRKPGA |
| SB07550 insert | 53 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| CD8 (H) | | TGGTATCAGCAGAAGCAGGGCAAGTCTCCTCAG |
| LIR1 (TM) | | CTGCTGGTGTTCAACGCCGAGACACTGCCTGAA |
| LIR1 (ICD) | | GGCGTGCCCAGCAGATTTTCTGGAACAGGCAGC |
| (GS)3 | | GGCACCCACTTCAGCCTGAGAATCAATAGCCTG |
| E34-V P2A PuroR | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAACCACG |
| | | ACGCCAGCGCCGCGACCACCAACACCGGCGCC |
| | | CACCATCGCGTTGCAGCCCCTGTCCCTGCGCCC |
| | | AGAGGCGTGCCGGCCAGCGGCGGGGGGCGCAG |
| | | TGCACACGAGGGGGCTGGACTTCGCCTGTGATG |
| | | TTATAGGGATCCTGGTGGCTGTCATACTCCTCTT |
| | | GCTCCTCTTGTTGCTGCTTTTTTTGATATTGCGC |
| | | CACAGACGGCAGGGAAAGCACTGGACTAGTAC |
| | | GCAGAGGAAAGCGGACTTCCAGCATCCCGCAG |
| | | GAGCCGTGGGGCCTGAACCCACTGATCGCGGCC |
| | | TTCAATGGAGGTCTAGCCCGGCGGCAGACGCAC |
| | | AAGAGGAAAACTTGTACGCAGCCGTTAAGCAC |
| | | ACCCAACCGGAGGACGGCGTTGAGATGGATAC |
| | | CCGCTCCCCTCACGATGAAGACCCTCAAGCAGT |
| | | CACTTACGCGGAAGTAAAGCATAGCCGCCCCA |
| | | GACGGGAAATGGCTAGCCCGCCGTCCCCCCTTA |
| | | GCGGGGAATTTCTGGACACTAAAGATAGGCAG |
| | | GCGGAAGAGGACCGCCAAATGGATACAGAGGC |
| | | GGCGGCAAGTGAAGCACCTCAAGACGTTACTTA |
| | | CGCTCAACTTCACAGCCTTACCCTCAGGCGAGA |
| | | AGCGACTGAACCACCCCCTTCCCAAGAAGGGC |
| | | CAAGCCCAGCGGTTCCTTCTATCTATGCTACTCT |
| | | TGCTATTCACGGTAGCGGCTCCGGCTCCATTAC |
| | | AATTAGAGCCGCCTTCCTGGAAAAAGAGAACA |
| | | CAGCCCTGAGAACCGAGGTGGCCGAACTCGAG |
| | | AAAGAAGTGGGCCGCTGCGAGAACATCGTGTC |
| | | TAAGTACGAGACAAGATACGGGCCCCTGGGAA |
| | | GCGGAGCTACTAACTTCAGCCTGCTGAAGCAGG |
| | | CTGGAGACGTGGAGGAGAACCCTGGACCTATG |
| | | ACCGAGTACAAGCCCACGGTGCGCCTCGCCACC |
| | | CGCGACGACGTCCCCAGGGCCGTACGCACCCTC |
| | | GCCGCCGCGTTCGCCGACTACCCCGCCACGCGC |
| | | CACACCGTCGATCCGGACCGCCACATCGAGCG |
| | | GGTCACCGAGCTGCAAGAACTCTTCCTCACGCG |
| | | CGTCGGGCTCGACATCGGCAAGGTGTGGGTCGC |
| | | GGACGACGGCGCCGCGGTGGCGGTCTGGACCA |
| | | CGCCGGAGAGCGTCGAAGCGGGGGCGGTGTTC |
| | | GCCGAGATCGGCCCGCGCATGGCCGAGTTGAG |
| | | CGGTTCCCGGCTGGCCGCGCAGCAACAGATGG |
| | | AAGGCCTCCTGGCGCCGCACCGGCCCAAGGAG |
| | | CCCGCGTGGTTCCTGGCCACCGTCGGCGTCTCG |
| | | CCCGACCACCAGGGCAAGGGTCTGGGCAGCGC |
| | | CGTCGTGCTCCCCGGAGTGGAGGCGGCCGAGC |
| | | GCGCCGGGGTGCCCGCCTTCCTGGAGACCTCCG |
| | | CGCCCCGCAACCTCCCCTTCTACGAGCGGCTCG |
| | | GCTTCACCGTCACCGCCGACGTCGAGGTGCCCG |
| | | AAGGACCGCGCACCTGGTGCATGACCCGCAAG |
| | | CCCGGTGCC |
| SB07550 insert | 54 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| CD8 (H) | | FTFSNSGMSWVRQTPDKRLEWVASISDGGLYTHY |
| LIR1 (TM) | | PDSVKGRFTISRDNGKSTLYLQMSSLRSEDTAIYY |
| LIR1 (ICD) | | CARQGVRPFFDYWGQGTTLTVSSGKPIPNPLLGL |
| (GS)3 | | DSTNGAATTTPAPRPPTPAPTIALQPLSLRPEACRP |
| E34-V P2A PuroR | | AAGGAVHTRGLDFACDVIGILVAVILLLLLLLLLF |
| | | LILRHRRQGKHWTSTQRKADFQHPAGAVGPEPTD |
| | | RGLQWRSSPAADAQEENLYAAVKHTQPEDGVEM |
| | | DTRSPHDEDPQAVTYAEVKHSRPRREMASPPSPL |
| | | SGEFLDTKDRQAEEDRQMDTEAAASEAPQDVTY |
| | | AQLHSLTLRREATEPPPSQEGPSPAVPSIYATLAIH |
| | | GSGSGSITIRAAFLEKENTALRTEVAELEKEVGRC |
| | | ENIVSKYETRYGPLGSGATNFSLLKQAGDVEENP |
| | | GPMTEYKPTVRLATRDDVPRAVRTLAAAFADYP |
| | | ATRHTVDPDRHIERVTELQELFLTRVGLDIGKVW |
| | | VADDGAAVAVWTTPESVEAGAVFAEIGPRMAEL |
| | | SGSRLAAQQQMEGLLAPHRPKEPAWFLATVGVSP |
| | | DHQGKGLGSAVVLPGVEAAERAGVPAFLETSAPR |
| | | NLPFYERLGFTVTADVEVPEGPRTWCMTRKPGA |
| SB07551 insert | 55 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| CD8 (H) | | TGGTATCAGCAGAAGCAGGGCAAGTCTCCTCAG |
| LIR1 (TM) | | CTGCTGGTGTTCAACGCCGAGACACTGCCTGAA |
| LIR1 (ICD) | | GGCGTGCCCAGCAGATTTTCTGGAACAGGCAGC |
| (GS)3 E34-N | | GGCACCCACTTCAGCCTGAGAATCAATAGCCTG |
| P2A PuroR | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAACCACG |
| | | ACGCCAGCGCCGCGACCACCAACACCGGCGCC |
| | | CACCATCGCGTTGCAGCCCCTGTCCCTGCGCCC |
| | | AGAGGCGTGCCGGCCAGCGGCGGGGGGCGCAG |
| | | TGCACACGAGGGGGCTGGACTTCGCCTGTGATG |
| | | TTATAGGGATCCTGGTGGCTGTCATACTCCTCTT |
| | | GCTCCTCTTGTTGCTGCTTTTTTTGATATTGCGC |
| | | CACAGACGGCAGGGAAAGCACTGGACTAGTAC |
| | | GCAGAGGAAAGCGGACTTCCAGCATCCCGCAG |
| | | GAGCCGTGGGGCCTGAACCCACTGATCGCGGCC |
| | | TTCAATGGAGGTCTAGCCCGGCGGCAGACGCAC |
| | | AAGAGGAAAACTTGTACGCAGCCGTTAAGCAC |
| | | ACCCAACCGGAGGACGGCGTTGAGATGGATAC |
| | | CCGCTCCCCTCACGATGAAGACCCTCAAGCAGT |
| | | CACTTACGCGGAAGTAAAGCATAGCCGCCCCA |
| | | GACGGGAAATGGCTAGCCCGCCGTCCCCCCTTA |
| | | GCGGGGAATTTCTGGACACTAAAGATAGGCAG |
| | | GCGGAAGAGGACCGCCAAATGGATACAGAGGC |
| | | GGCGGCAAGTGAAGCACCTCAAGACGTTACTTA |
| | | CGCTCAACTTCACAGCCTTACCCTCAGGCGAGA |
| | | AGCGACTGAACCACCCCCTTCCCAAGAAGGGC |
| | | CAAGCCCAGCGGTTCCTTCTATCTATGCTACTCT |
| | | TGCTATTCACGGATCTGGCTCTGGATCCATTAC |
| | | AATCAGAGCCGCCTTCCTCGAGAAAGAGAACA |
| | | CAGCCCTGAGAACCGAGAACGCCGAGCTGGAA |
| | | AAAGAAGTGGGCCGCTGCGAGAACATCGTGTC |
| | | TAAGTACGAGACAAGATACGGCCCACTCGGAA |
| | | GCGGAGCTACTAACTTCAGCCTGCTGAAGCAGG |
| | | CTGGAGACGTGGAGGAGAACCCTGGACCTATG |
| | | ACCGAGTACAAGCCCACGGTGCGCCTCGCCACC |
| | | CGCGACGACGTCCCCAGGGCCGTACGCACCCTC |
| | | GCCGCCGCGTTCGCCGACTACCCCGCCACGCGC |
| | | CACACCGTCGATCCGGACCGCCACATCGAGCG |
| | | GGTCACCGAGCTGCAAGAACTCTTCCTCACGCG |
| | | CGTCGGGCTCGACATCGGCAAGGTGTGGGTCGC |
| | | GGACGACGGCGCCGCGGTGGCGGTCTGGACCA |
| | | CGCCGGAGAGCGTCGAAGCGGGGGCGGTGTTC |
| | | GCCGAGATCGGCCCGCGCATGGCCGAGTTGAG |
| | | CGGTTCCCGGCTGGCCGCGCAGCAACAGATGG |
| | | AAGGCCTCCTGGCGCCGCACCGGCCCAAGGAG |
| | | CCCGCGTGGTTCCTGGCCACCGTCGGCGTCTCG |
| | | CCCGACCACCAGGGCAAGGGTCTGGGCAGCGC |
| | | CGTCGTGCTCCCCGGAGTGGAGGCGGCCGAGC |
| | | GCGCCGGGGTGCCCGCCTTCCTGGAGACCTCCG |
| | | CGCCCCGCAACCTCCCCTTCTACGAGCGGCTCG |
| | | GCTTCACCGTCACCGCCGACGTCGAGGTGCCCG |
| | | AAGGACCGCGCACCTGGTGCATGACCCGCAAG |
| | | CCCGGTGCC |
| SB07551 insert | 56 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| CD8 (H) | | FTFSNSGMSWVRQTPDKRLEWVASISDGGLYTHY |
| LIR1 (TM) | | PDSVKGRFTISRDNGKSTLYLQMSSLRSEDTAIYY |
| LIR1 (ICD) | | CARQGVRPFFDYWGQGTTLTVSSGKPIPNPLLGL |
| (GS)3 E34-N | | DSTNGAATTTPAPRPPTPAPTIALQPLSLRPEACRP |
| P2A PuroR | | AAGGAVHTRGLDFACDVIGILVAVILLLLLLLLLF |
| | | LILRHRRQGKHWTSTQRKADFQHPAGAVGPEPTD |
| | | RGLQWRSSPAADAQEENLYAAVKHTQPEDGVEM |
| | | DTRSPHDEDPQAVTYAEVKHSRPRREMASPPSPL |
| | | SGEFLDTKDRQAEEDRQMDTEAAASEAPQDVTY |
| | | AQLHSLTLRREATEPPPSQEGPSPAVPSIYATLAIH |
| | | GSGSGSITIRAAFLEKENTALRTENAELEKEVGRC |
| | | ENIVSKYETRYGPLGSGATNFSLLKQAGDVEENP |
| | | GPMTEYKPTVRLATRDDVPRAVRTLAAAFADYP |
| | | ATRHTVDPDRHIERVTELQELFLTRVGLDIGKVW |
| | | VADDGAAVAVWTTPESVEAGAVFAEIGPRMAEL |
| | | SGSRLAAQQQMEGLLAPHRPKEPAWFLATVGVSP |
| | | DHQGKGLGSAVVLPGVEAAERAGVPAFLETSAPR |
| | | NLPFYERLGFTVTADVEVPEGPRTWCMTRKPGA |
| SB07552 insert | 57 | ATGGCCCTGCCTGTGACAGCTCTGCTGCTGCCT |
| sequence: | (nucleic acid) | CTGGCCCTGCTGCTGCATGCTGCTAGACCTGAC |
| CD8(SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| CD94 (H)-rev | | ATGGTATCAGCAGAAGCCTGGCAAGGCCCCTA |
| CD28 (TM) | | AGCTGCTGATCTACTGGACCAGCACCAGACACA |
| CD28 (ICD) z | | CAGGCGTGCCCAGCAGATTTTCTGGCAGCGGCT |
| | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAGGCGTTTGGAAGGAGCAGTGTTCGTGT |
| | | TGTGACAGTGACAAGCAGCTGGAGATCAATCC |
| | | GGGACCAACTTTTGCGCCTGAAATTTCTCTGAA |
| | | GACCTTCAGCAACAAGTTTTGGGTGCTGGTGGT |
| | | GGTTGGTGGAGTCCTGGCTTGCTATAGCTTGCT |
| | | AGTAACAGTGGCCTTTATTATTTTCTGGGTGAG |
| | | GAGTAAGAGGAGCAGGCTCCTGCACAGTGACT |
| | | ACATGAACATGACTCCCCGCCGCCCCGGGCCCA |
| | | CCCGCAAGCATTACCAGCCCTATGCCCCACCAC |
| | | GCGACTTCGCAGCCTATCGCTCCAGAGTGAAGT |
| | | TCAGCAGGAGCGCAGACGCCCCCGCGTACAAG |
| | | CAGGGCCAGAACCAGCTCTATAACGAGCTCAAT |
| | | CTAGGACGAAGAGAGGAGTACGATGTTTTGGA |
| | | CAAGAGACGTGGCCGGGACCCTGAGATGGGGG |
| | | GAAAGCCGAGAAGGAAGAACCCTCAGGAAGGC |
| | | CTGTACAATGAACTGCAGAAAGATAAGATGGC |
| | | GGAGGCCTACAGTGAGATTGGGATGAAAGGCG |
| | | AGCGCCGGAGGGGCAAGGGGCACGATGGCCTT |
| | | TACCAGGGTCTCAGTACAGCCACCAAGGACACC |
| | | TACGACGCCCTTCACATGCAGGCCCTGCCCCCT |
| | | CGC |
| SB07552 insert | 58 | MALPVTALLLPLALLLHAARPDIQLTQSPSSLSAS |
| sequence: | (amino acid) | VGDRVTITCKASQDVGTSVAWYQQKPGKAPKLLI |
| CD8(SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| CD94 (H)-rev | | WMSWVRQAPGKGLEWIGEIHPDSSTINYAPSLKD |
| CD28 (TM) | | RFTISRDNAKNTLFLQMDSLRPEDTGVYFCASLYF |
| CD28 (ICD) z | | GFPWFAYWGQGTPVTVSSEQKLISEEDLNGAAGV |
| | | WKEQCSCCDSDKQLEINPGPTFAPEISLKTFSNKF |
| | | WVLVVVGGVLACYSLLVTVAFIIFWVRSKRSRLL |
| | | HSDYMNMTPRRPGPTRKHYQPYAPPRDFAAYRS |
| | | RVKFSRSADAPAYKQGQNQLYNELNLGRREEYD |
| | | VLDKRRGRDPEMGGKPRRKNPQEGLYNELQKDK |
| | | MAEAYSEIGMKGERRRGKGHDGLYQGLSTATKD |
| | | TYDALHMQALPPR |
| SB07553 insert | 59 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| NKG2A (H)-rev | | ATGGTATCAGCAGAAGCCTGGCAAGGCCCCTA |
| CD28 (TM) | | AGCTGCTGATCTACTGGACCAGCACCAGACACA |
| CD28 (ICD) z | | CAGGCGTGCCCAGCAGATTTTCTGGCAGCGGCT |
| | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAGGGTGCCATCGCGCCAAGCAGACGCG |
| | | CACCAACCTCAGTTCTAACAACCACCGCCAAAT |
| | | TCTAACAAGCCCCTTTTGGGTGCTGGTGGTGGT |
| | | TGGTGGAGTCCTGGCTTGCTATAGCTTGCTAGT |
| | | AACAGTGGCCTTTATTATTTTCTGGGTGAGGAG |
| | | TAAGAGGAGCAGGCTCCTGCACAGTGACTACAT |
| | | GAACATGACTCCCCGCCGCCCCGGGCCCACCCG |
| | | CAAGCATTACCAGCCCTATGCCCCACCACGCGA |
| | | CTTCGCAGCCTATCGCTCCAGAGTGAAGTTCAG |
| | | CAGGAGCGCAGACGCCCCCGCGTACAAGCAGG |
| | | GCCAGAACCAGCTCTATAACGAGCTCAATCTAG |
| | | GACGAAGAGAGGAGTACGATGTTTTGGACAAG |
| | | AGACGTGGCCGGGACCCTGAGATGGGGGGAAA |
| | | GCCGAGAAGGAAGAACCCTCAGGAAGGCCTGT |
| | | ACAATGAACTGCAGAAAGATAAGATGGCGGAG |
| | | GCCTACAGTGAGATTGGGATGAAAGGCGAGCG |
| | | CCGGAGGGGCAAGGGGCACGATGGCCTTTACC |
| | | AGGGTCTCAGTACAGCCACCAAGGACACCTAC |
| | | GACGCCCTTCACATGCAGGCCCTGCCCCCTCGC |
| SB07553 insert | 60 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| NKG2A (H)-rev | | WMSWVRQAPGKGLEWIGEIHPDSSTINYAPSLKD |
| CD28 (TM) | | RFTISRDNAKNTLFLQMDSLRPEDTGVYFCASLYF |
| CD28 (ICD) z | | GFPWFAYWGQGTPVTVSSEQKLISEEDLNGAAGC |
| | | HRAKQTRTNLSSNNHRQILTSPFWVLVVVGGVLA |
| | | CYSLLVTVAFIIFWVRSKRSRLLHSDYMNMTPRR |
| | | PGPTRKHYQPYAPPRDFAAYRSRVKFSRSADAPA |
| | | YKQGQNQLYNELNLGRREEYDVLDKRRGRDPEM |
| | | GGKPRRKNPQEGLYNELQKDKMAEAYSEIGMKG |
| | | ERRRGKGHDGLYQGLSTATKDTYDALHMQALPP |
| | | R |
| SB07554 insert | 61 | ATGGCCCTGCCTGTGACAGCTCTGCTGCTGCCT |
| sequence: | (nucleic acid) | CTGGCCCTGCTGCTGCATGCTGCTAGACCTGAC |
| CD8 (SS) | | ATCCAGCTGACACAGAGCCCTAGCAGCCTGTCT |
| aCEA | | GCCTCTGTGGGCGACAGAGTGACCATCACATGC |
| myc | | AAGGCCTCTCAGGACGTGGGCACAAGCGTGGC |
| NKG2C (H)-rcv | | ATGGTATCAGCAGAAGCCTGGCAAGGCCCCTA |
| CD28 (TM) | | AGCTGCTGATCTACTGGACCAGCACCAGACACA |
| CD28 (ICD) z | | CAGGCGTGCCCAGCAGATTTTCTGGCAGCGGCT |
| | | CTGGCACCGACTTCACCTTCACCATAAGCAGCC |
| | | TGCAGCCTGAGGATATCGCCACCTACTACTGCC |
| | | AGCAGTACAGCCTGTACAGAAGCTTCGGCCAG |
| | | GGCACCAAGGTGGAAATCAAAGGCGGATCTGG |
| | | AAGCGGCGGTTCTGGATCTGGTGGAAGCGGATC |
| | | TGAGGTGCAGCTGGTGGAATCTGGTGGCGGAGT |
| | | TGTGCAGCCTGGCAGATCTCTGAGACTGAGCTG |
| | | TAGCGCCAGCGGCTTCGATTTCACCACCTACTG |
| | | GATGAGCTGGGTCCGACAGGCCCCTGGCAAAG |
| | | GACTGGAATGGATCGGCGAGATTCACCCCGAC |
| | | AGCAGCACCATCAATTACGCCCCTAGCCTGAAG |
| | | GACCGGTTCACCATCTCCAGAGACAACGCCAAG |
| | | AATACCCTGTTCCTGCAGATGGACAGCCTCCGG |
| | | CCTGAAGATACCGGCGTGTACTTTTGCGCCAGC |
| | | CTGTATTTCGGCTTCCCTTGGTTTGCCTACTGGG |
| | | GCCAGGGAACACCTGTGACCGTTAGCTCTGAAC |
| | | AAAAACTCATCTCAGAAGAAGATCTGAATGGG |
| | | GCCGCAGGATGTCACCGCGCCAAACAGACTCG |
| | | CACCAACCCCTCTTCGAACAACCAGGAGCTGTT |
| | | CCCCATCTTTTGGGTGCTGGTGGTGGTTGGTGG |
| | | AGTCCTGGCTTGCTATAGCTTGCTAGTAACAGT |
| | | GGCCTTTATTATTTTCTGGGTGAGGAGTAAGAG |
| | | GAGCAGGCTCCTGCACAGTGACTACATGAACAT |
| | | GACTCCCCGCCGCCCCGGGCCCACCCGCAAGCA |
| | | TTACCAGCCCTATGCCCCACCACGCGACTTCGC |
| | | AGCCTATCGCTCCAGAGTGAAGTTCAGCAGGAG |
| | | CGCAGACGCCCCCGCGTACAAGCAGGGCCAGA |
| | | ACCAGCTCTATAACGAGCTCAATCTAGGACGAA |
| | | GAGAGGAGTACGATGTTTTGGACAAGAGACGT |
| | | GGCCGGGACCCTGAGATGGGGGGAAAGCCGAG |
| | | AAGGAAGAACCCTCAGGAAGGCCTGTACAATG |
| | | AACTGCAGAAAGATAAGATGGCGGAGGCCTAC |
| | | AGTGAGATTGGGATGAAAGGCGAGCGCCGGAG |
| | | GGGCAAGGGGCACGATGGCCTTTACCAGGGTCT |
| | | CAGTACAGCCACCAAGGACACCTACGACGCCC |
| | | TTCACATGCAGGCCCTGCCCCCTCGC |
| SB07554 insert | 62 | MALPVTALLLPLALLLHAARPDIQLTQSPSSLSAS |
| sequence: | (amino acid) | VGDRVTITCKASQDVGTSVAWYQQKPGKAPKLLI |
| CD8 (SS) | | YWTSTRHTGVPSRFSGSGSGTDFTFTISSLQPEDIA |
| aCEA | | TYYCQQYSLYRSFGQGTKVEIKGGSGSGGSGSGG |
| myc | | SGSEVQLVESGGGVVQPGRSLRLSCSASGFDFTTY |
| NKG2C (H)-rev | | WMSWVRQAPGKGLEWIGEIHPDSSTINYAPSLKD |
| CD28 (TM) | | RFTISRDNAKNTLFLQMDSLRPEDTGVYFCASLYF |
| CD28 (ICD) z | | GFPWFAYWGQGTPVTVSSEQKLISEEDLNGAAGC |
| | | HRAKQTRTNPSSNNQELFPIFWVLVVVGGVLACY |
| | | SLLVTVAFIIFWVRSKRSRLLHSDYMNMTPRRPGP |
| | | TRKHYQPYAPPRDFAAYRSRVKFSRSADAPAYKQ |
| | | GQNQLYNELNLGRREEYDVLDKRRGRDPEMGGK |
| | | PRRKNPQEGLYNELQKDKMAEAYSEIGMKGERR |
| | | RGKGHDGLYQGLSTATKDTYDALHMQALPPR |
| SB07555 insert | 63 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| CD94 (H)-rev | | TGGTATCAGCAGAAGCAGGGCAAGTCTCCTCAG |
| LIR1 (TM) | | CTGCTGGTGTTCAACGCCGAGACACTGCCTGAA |
| LIR1 (ICD) | | GGCGTGCCCAGCAGATTTTCTGGAACAGGCAGC |
| P2A PuroR | | GGCACCCACTTCAGCCTGAGAATCAATAGCCTG |
| | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAGGCGTT |
| | | TGGAAGGAGCAGTGTTCGTGTTGTGACAGTGAC |
| | | AAGCAGCTGGAGATCAATCCGGGACCAACTTTT |
| | | GCGCCTGAAATTTCTCTGAAGACCTTCAGCAAC |
| | | AAGGTTATAGGGATCCTGGTGGCTGTCATACTC |
| | | CTCTTGCTCCTCTTGTTGCTGCTTTTTTTGATATT |
| | | GCGCCACAGACGGCAGGGAAAGCACTGGACTA |
| | | GTACGCAGAGGAAAGCGGACTTCCAGCATCCC |
| | | GCAGGAGCCGTGGGGCCTGAACCCACTGATCG |
| | | CGGCCTTCAATGGAGGTCTAGCCCGGCGGCAGA |
| | | CGCACAAGAGGAAAACTTGTACGCAGCCGTTA |
| | | AGCACACCCAACCGGAGGACGGCGTTGAGATG |
| | | GATACCCGCTCCCCTCACGATGAAGACCCTCAA |
| | | GCAGTCACTTACGCGGAAGTAAAGCATAGCCG |
| | | CCCCAGACGGGAAATGGCTAGCCCGCCGTCCCC |
| | | CCTTAGCGGGGAATTTCTGGACACTAAAGATAG |
| | | GCAGGCGGAAGAGGACCGCCAAATGGATACAG |
| | | AGGCGGCGGCAAGTGAAGCACCTCAAGACGTT |
| | | ACTTACGCTCAACTTCACAGCCTTACCCTCAGG |
| | | CGAGAAGCGACTGAACCACCCCCTTCCCAAGA |
| | | AGGGCCAAGCCCAGCGGTTCCTTCTATCTATGC |
| | | TACTCTTGCTATTCACGGAAGCGGAGCTACTAA |
| | | CTTCAGCCTGCTGAAGCAGGCTGGAGACGTGGA |
| | | GGAGAACCCTGGACCTATGACCGAGTACAAGC |
| | | CCACGGTGCGCCTCGCCACCCGCGACGACGTCC |
| | | CCAGGGCCGTACGCACCCTCGCCGCCGCGTTCG |
| | | CCGACTACCCCGCCACGCGCCACACCGTCGATC |
| | | CGGACCGCCACATCGAGCGGGTCACCGAGCTG |
| | | CAAGAACTCTTCCTCACGCGCGTCGGGCTCGAC |
| | | ATCGGCAAGGTGTGGGTCGCGGACGACGGCGC |
| | | CGCGGTGGCGGTCTGGACCACGCCGGAGAGCG |
| | | TCGAAGCGGGGGCGGTGTTCGCCGAGATCGGC |
| | | CCGCGCATGGCCGAGTTGAGCGGTTCCCGGCTG |
| | | GCCGCGCAGCAACAGATGGAAGGCCTCCTGGC |
| | | GCCGCACCGGCCCAAGGAGCCCGCGTGGTTCCT |
| | | GGCCACCGTCGGCGTCTCGCCCGACCACCAGGG |
| | | CAAGGGTCTGGGCAGCGCCGTCGTGCTCCCCGG |
| | | AGTGGAGGCGGCCGAGCGCGCCGGGGTGCCCG |
| | | CCTTCCTGGAGACCTCCGCGCCCCGCAACCTCC |
| | | CCTTCTACGAGCGGCTCGGCTTCACCGTCACCG |
| | | CCGACGTCGAGGTGCCCGAAGGACCGCGCACC |
| | | TGGTGCATGACCCGCAAGCCCGGTGCC |
| SB07555 insert | 64 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| CD94 (H)-rev | | FTFSNSGMSWVRQTPDKRLEWVASISDGGLYTHY |
| LIR1 (TM) | | PDSVKGRFTISRDNGKSTLYLQMSSLRSEDTAIYY |
| LIR1 (ICD) | | CARQGVRPFFDYWGQGTTLTVSSGKPIPNPLLGL |
| P2A PuroR | | DSTNGAAGVWKEQCSCCDSDKQLEINPGPTFAPEI |
| | | SLKTFSNKVIGILVAVILLLLLLLLLFLILRHRRQG |
| | | KHWTSTQRKADFQHPAGAVGPEPTDRGLQWRSS |
| | | PAADAQEENLYAAVKHTQPEDGVEMDTRSPHDE |
| | | DPQAVTYAEVKHSRPRREMASPPSPLSGEFLDTK |
| | | DRQAEEDRQMDTEAAASEAPQDVTYAQLHSLTL |
| | | RREATEPPPSQEGPSPAVPSIYATLAIHGSGATNFS |
| | | LLKQAGDVEENPGPMTEYKPTVRLATRDDVPRA |
| | | VRTLAAAFADYPATRHTVDPDRHIERVTELQELF |
| | | LTRVGLDIGKVWVADDGAAVAVWTTPESVEAGA |
| | | VFAEIGPRMAELSGSRLAAQQQMEGLLAPHRPKE |
| | | PAWFLATVGVSPDHQGKGLGSAVVLPGVEAAER |
| | | AGVPAFLETSAPRNLPFYERLGFTVTADVEVPEGP |
| | | RTWCMTRKPGA |
| SB07556 insert | 65 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| NKG2A (H)-rcv | | TGGTATCAGCAGAAGCAGGGCAAGTCTCCTCAG |
| LIR1 (TM) | | CTGCTGGTGTTCAACGCCGAGACACTGCCTGAA |
| LIR1 (ICD) | | GGCGTGCCCAGCAGATTTTCTGGAACAGGCAGC |
| P2A PuroR | | GGCACCCACTTCAGCCTGAGAATCAATAGCCTG |
| | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAGGGTGC |
| | | CATCGCGCCAAGCAGACGCGCACCAACCTCAG |
| | | TTCTAACAACCACCGCCAAATTCTAACAAGCCC |
| | | CGTTATAGGGATCCTGGTGGCTGTCATACTCCT |
| | | CTTGCTCCTCTTGTTGCTGCTTTTTTTGATATTGC |
| | | GCCACAGACGGCAGGGAAAGCACTGGACTAGT |
| | | ACGCAGAGGAAAGCGGACTTCCAGCATCCCGC |
| | | AGGAGCCGTGGGGCCTGAACCCACTGATCGCG |
| | | GCCTTCAATGGAGGTCTAGCCCGGCGGCAGACG |
| | | CACAAGAGGAAAACTTGTACGCAGCCGTTAAG |
| | | CACACCCAACCGGAGGACGGCGTTGAGATGGA |
| | | TACCCGCTCCCCTCACGATGAAGACCCTCAAGC |
| | | AGTCACTTACGCGGAAGTAAAGCATAGCCGCCC |
| | | CAGACGGGAAATGGCTAGCCCGCCGTCCCCCCT |
| | | TAGCGGGGAATTTCTGGACACTAAAGATAGGC |
| | | AGGCGGAAGAGGACCGCCAAATGGATACAGAG |
| | | GCGGCGGCAAGTGAAGCACCTCAAGACGTTAC |
| | | TTACGCTCAACTTCACAGCCTTACCCTCAGGCG |
| | | AGAAGCGACTGAACCACCCCCTTCCCAAGAAG |
| | | GGCCAAGCCCAGCGGTTCCTTCTATCTATGCTA |
| | | CTCTTGCTATTCACGGAAGCGGAGCTACTAACT |
| | | TCAGCCTGCTGAAGCAGGCTGGAGACGTGGAG |
| | | GAGAACCCTGGACCTATGACCGAGTACAAGCC |
| | | CACGGTGCGCCTCGCCACCCGCGACGACGTCCC |
| | | CAGGGCCGTACGCACCCTCGCCGCCGCGTTCGC |
| | | CGACTACCCCGCCACGCGCCACACCGTCGATCC |
| | | GGACCGCCACATCGAGCGGGTCACCGAGCTGC |
| | | AAGAACTCTTCCTCACGCGCGTCGGGCTCGACA |
| | | TCGGCAAGGTGTGGGTCGCGGACGACGGCGCC |
| | | GCGGTGGCGGTCTGGACCACGCCGGAGAGCGT |
| | | CGAAGCGGGGGCGGTGTTCGCCGAGATCGGCC |
| | | CGCGCATGGCCGAGTTGAGCGGTTCCCGGCTGG |
| | | CCGCGCAGCAACAGATGGAAGGCCTCCTGGCG |
| | | CCGCACCGGCCCAAGGAGCCCGCGTGGTTCCTG |
| | | GCCACCGTCGGCGTCTCGCCCGACCACCAGGGC |
| | | AAGGGTCTGGGCAGCGCCGTCGTGCTCCCCGGA |
| | | GTGGAGGCGGCCGAGCGCGCCGGGGTGCCCGC |
| | | CTTCCTGGAGACCTCCGCGCCCCGCAACCTCCC |
| | | CTTCTACGAGCGGCTCGGCTTCACCGTCACCGC |
| | | CGACGTCGAGGTGCCCGAAGGACCGCGCACCT |
| | | GGTGCATGACCCGCAAGCCCGGTGCC |
| SB07556 insert | 66 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| NKG2A (H)-rev | | FTFSNSGMSWVRQTPDKRLEWVASISDGGLYTHY |
| LIR1 (TM) | | PDSVKGRFTISRDNGKSTLYLQMSSLRSEDTAIYY |
| LIR1 (ICD) | | CARQGVRPFFDYWGQGTTLTVSSGKPIPNPLLGL |
| P2A PuroR | | DSTNGAAGCHRAKQTRTNLSSNNHRQILTSPVIGI |
| | | LVAVILLLLLLLLLFLILRHRRQGKHWTSTQRKAD |
| | | FQHPAGAVGPEPTDRGLQWRSSPAADAQEENLY |
| | | AAVKHTQPEDGVEMDTRSPHDEDPQAVTYAEVK |
| | | HSRPRREMASPPSPLSGEFLDTKDRQAEEDRQMD |
| | | TEAAASEAPQDVTYAQLHSLTLRREATEPPPSQEG |
| | | PSPAVPSIYATLAIHGSGATNFSLLKQAGDVEENP |
| | | GPMTEYKPTVRLATRDDVPRAVRTLAAAFADYP |
| | | ATRHTVDPDRHIERVTELQELFLTRVGLDIGKVW |
| | | VADDGAAVAVWTTPESVEAGAVFAEIGPRMAEL |
| | | SGSRLAAQQQMEGLLAPHRPKEPAWFLATVGVSP |
| | | DHQGKGLGSAVVLPGVEAAERAGVPAFLETSAPR |
| | | NLPFYERLGFTVTADVEVPEGPRTWCMTRKPGA |
| SB07557 insert | 67 | ATGGCCTTACCAGTGACCGCCTTGCTCCTGCCG |
| sequence: | (nucleic acid) | CTGGCCTTGCTGCTCCACGCCGCCAGGCCGGAC |
| CD8 (SS) | | ATCCAGATGACACAGTCTCCAGCCAGCCTGTCT |
| aVSIG2 | | GCCTCTGTGGGAGAGACAGTGACCATGACCTGT |
| V5 | | CGGGCCAGCGAGAACATCTACAGCTACCTGGCC |
| NKG2C (H)-rev | | TGGTATCAGCAGAAGCAGGGCAAGTCTCCTCAG |
| LIR1 (TM) | | CTGCTGGTGTTCAACGCCGAGACACTGCCTGAA |
| LIR1 (ICD) | | GGCGTGCCCAGCAGATTTTCTGGAACAGGCAGC |
| P2A PuroR | | GGCACCCACTTCAGCCTGAGAATCAATAGCCTG |
| | | CAGCCTGAGGACTTCGGCAGCTACTACTGCCAG |
| | | CACCACTACGTGATCCCTTGGACCTTTGGCGGA |
| | | GGCACCAAGCTGGAAATCAAGGGCAGCACAAG |
| | | CGGCTCTGGAAAACCTGGATCTGGCGAGGGCTC |
| | | TACCAAGGGCGAGGTGCAGATGGTTGAGTCTG |
| | | GCGGCGATCTGGTTAAGCCTGGCGGAAGCCTGA |
| | | AGCTGTCTTGTGCCGCCAGCGGCTTCACCTTCA |
| | | GCAATAGCGGCATGAGCTGGGTCCGACAGACC |
| | | CCTGACAAGAGACTGGAATGGGTCGCCAGCAT |
| | | CTCTGACGGCGGCCTGTACACACACTACCCCGA |
| | | TTCTGTGAAGGGCAGATTCACCATCAGCAGAGA |
| | | CAACGGCAAGAGCACCCTGTACCTGCAGATGA |
| | | GCAGCCTGAGAAGCGAGGACACCGCCATCTAC |
| | | TACTGCGCCAGACAGGGCGTCAGACCCTTCTTC |
| | | GATTATTGGGGCCAGGGCACCACACTGACCGTG |
| | | TCATCTGGGAAGCCTATCCCGAACCCTCTGTTG |
| | | GGTCTCGATAGTACCAATGGGGCCGCAGGATGT |
| | | CACCGCGCCAAACAGACTCGCACCAACCCCTCT |
| | | TCGAACAACCAGGAGCTGTTCCCCATCGTTATA |
| | | GGGATCCTGGTGGCTGTCATACTCCTCTTGCTCC |
| | | TCTTGTTGCTGCTTTTTTTGATATTGCGCCACAG |
| | | ACGGCAGGGAAAGCACTGGACTAGTACGCAGA |
| | | GGAAAGCGGACTTCCAGCATCCCGCAGGAGCC |
| | | GTGGGGCCTGAACCCACTGATCGCGGCCTTCAA |
| | | TGGAGGTCTAGCCCGGCGGCAGACGCACAAGA |
| | | GGAAAACTTGTACGCAGCCGTTAAGCACACCCA |
| | | ACCGGAGGACGGCGTTGAGATGGATACCCGCT |
| | | CCCCTCACGATGAAGACCCTCAAGCAGTCACTT |
| | | ACGCGGAAGTAAAGCATAGCCGCCCCAGACGG |
| | | GAAATGGCTAGCCCGCCGTCCCCCCTTAGCGGG |
| | | GAATTTCTGGACACTAAAGATAGGCAGGCGGA |
| | | AGAGGACCGCCAAATGGATACAGAGGCGGCGG |
| | | CAAGTGAAGCACCTCAAGACGTTACTTACGCTC |
| | | AACTTCACAGCCTTACCCTCAGGCGAGAAGCGA |
| | | CTGAACCACCCCCTTCCCAAGAAGGGCCAAGCC |
| | | CAGCGGTTCCTTCTATCTATGCTACTCTTGCTAT |
| | | TCACGGAAGCGGAGCTACTAACTTCAGCCTGCT |
| | | GAAGCAGGCTGGAGACGTGGAGGAGAACCCTG |
| | | GACCTATGACCGAGTACAAGCCCACGGTGCGCC |
| | | TCGCCACCCGCGACGACGTCCCCAGGGCCGTAC |
| | | GCACCCTCGCCGCCGCGTTCGCCGACTACCCCG |
| | | CCACGCGCCACACCGTCGATCCGGACCGCCACA |
| | | TCGAGCGGGTCACCGAGCTGCAAGAACTCTTCC |
| | | TCACGCGCGTCGGGCTCGACATCGGCAAGGTGT |
| | | GGGTCGCGGACGACGGCGCCGCGGTGGCGGTC |
| | | TGGACCACGCCGGAGAGCGTCGAAGCGGGGGC |
| | | GGTGTTCGCCGAGATCGGCCCGCGCATGGCCGA |
| | | GTTGAGCGGTTCCCGGCTGGCCGCGCAGCAACA |
| | | GATGGAAGGCCTCCTGGCGCCGCACCGGCCCA |
| | | AGGAGCCCGCGTGGTTCCTGGCCACCGTCGGCG |
| | | TCTCGCCCGACCACCAGGGCAAGGGTCTGGGCA |
| | | GCGCCGTCGTGCTCCCCGGAGTGGAGGCGGCCG |
| | | AGCGCGCCGGGGTGCCCGCCTTCCTGGAGACCT |
| | | CCGCGCCCCGCAACCTCCCCTTCTACGAGCGGC |
| | | TCGGCTTCACCGTCACCGCCGACGTCGAGGTGC |
| | | CCGAAGGACCGCGCACCTGGTGCATGACCCGC |
| | | AAGCCCGGTGCC |
| SB07557 insert | 68 | MALPVTALLLPLALLLHAARPDIQMTQSPASLSAS |
| sequence: | (amino acid) | VGETVTMTCRASENIYSYLAWYQQKQGKSPQLL |
| CD8 (SS) | | VFNAETLPEGVPSRFSGTGSGTHFSLRINSLQPEDF |
| aVSIG2 | | GSYYCQHHYVIPWTFGGGTKLEIKGSTSGSGKPG |
| V5 | | SGEGSTKGEVQMVESGGDLVKPGGSLKLSCAASG |
| NKG2C (H)-rcv | | FTFSNSGMSWVRQTPDKRLEWVASISDGGLYTHY |
| LIR1 (TM) | | PDSVKGRFTISRDNGKSTLYLQMSSLRSEDTAIYY |
| LIR1 (ICD) | | CARQGVRPFFDYWGQGTTLTVSSGKPIPNPLLGL |
| P2A PuroR | | DSTNGAAGCHRAKQTRTNPSSNNQELFPIVIGILV |
| | | AVILLLLLLLLLFLILRHRRQGKHWTSTQRKADFQ |
| | | HPAGAVGPEPTDRGLQWRSSPAADAQEENLYAA |
| | | VKHTQPEDGVEMDTRSPHDEDPQAVTYAEVKHS |
| | | RPRREMASPPSPLSGEFLDTKDRQAEEDRQMDTE |
| | | AAASEAPQDVTYAQLHSLTLRREATEPPPSQEGPS |
| | | PAVPSIYATLAIHGSGATNFSLLKQAGDVEENPGP |
| | | MTEYKPTVRLATRDDVPRAVRTLAAAFADYPAT |
| | | RHTVDPDRHIERVTELQELFLTRVGLDIGKVWVA |
| | | DDGAAVAVWTTPESVEAGAVFAEIGPRMAELSGS |
| | | RLAAQQQMEGLLAPHRPKEPAWFLATVGVSPDH |
| | | QGKGLGSAVVLPGVEAAERAGVPAFLETSAPRNL |
| | | PFYERLGFTVTADVEVPEGPRTWCMTRKPGA |
| CD94 (H) | 69 | AAGAACAGCTTCACCAAGCTGAGCATCGAGCCCGCCTTC |
| | (nucleic acid) | ACACCCGGACCTAATATCGAGCTGCAGAAGGACAGCGAC |
| | | TGCTGCAGCTGCCAAGAGAAATGGGTCGGA |
| CD94 (H) | 70 | KNSFTKLSIEPAFTPGPNIELQKDSDCCSCQEKWVG |
| | (amino acid) | |
| CD94 (extended H) | 71 | AAGAACAGCTTCACCAAGCTGAGCATCGAGCCCGCCTTC |
| | (nucleic acid) | ACACCCGGACCTAATATCGAGCTGCAGAAGGACAGCGAC |
| | | TGCTGCAGCTGCCAAGAGAAATGGGTCGGATACCGGTGC |
| | | AACTGCTACTTCATCAGCAGCGAGCAGAAAACCTGG |
| CD94 (extended H) | 72 | KNSFTKLSIEPAFTPGPNIELQKDSDCCSCQEKWVGYRCNCY |
| | (amino acid) | FISSEQKTW |
| NKG2A (H) | 73 | CCCAGCACACTGATCCAGCGGCACAACAACAGCAGCCTG |
| | (nucleic acid) | AACACCAGAACACAGAAGGCCCGGCACTGCGGC |
| NKG2A (H) | 74 | PSTLIQRHNNSSLNTRTQKARHCG |
| | (amino acid) | |
| NKG2C (H) | 75 | ATCCCATTCCTCGAGCAGAACAACAGCAGCCCCAACACA |
| | (nucleic acid) | AGAACCCAGAAGGCCAGACACTGCGGA |
| NKG2C (H) | 76 | IPFLEQNNSSPNTRTQKARHCG |
| | (amino acid) | |
| R34 (GS)3 | 77 | CTGGAAATCAGGGCCGCCTTCCTGGAAAAAGAGAACACA |
| | (nucleic acid) | GCCCTGCGGACAAGAGCCGCCGAGCTGAGAAAACGCGTG |
| | | GGCAGATGCCGGAACATCGTGTCTAAGTACGAGACAAGA |
| | | TACGGCCCTCTGGGCTCTGGTAGTGGATCT |
| R34 (GS)3 | 78 | LEIRAAFLEKENTALRTRAAELRKRVGRCRNIVSKYETRYGP |
| | (amino acid) | LGSGSGS |
| E34-I (GS)3 | 79 | ATTACAATTAGAGCCGCATTCCTCGAGAAAGAAAACACC |
| | (nucleic acid) | GCTCTGAGAACCGAGATTGCCGAACTCGAAAAAGAAGTG |
| | | GGGCGCTGCGAGAATATCGTGTCCAAATACGAAACCCGC |
| | | TACGGCCCACTCGGCTCTGGTAGTGGATCT |
| E34-I (GS)3 | 80 | ITIRAAFLEKENTALRTEIAELEKEVGRCENIVSKYETRYGPL |
| | (amino acid) | GSGSGS |
| E34-V (GS)3 | 81 | ATTACAATTAGAGCCGCCTTCCTGGAAAAAGAGAACACA |
| | (nucleic acid) | GCCCTGAGAACCGAGGTGGCCGAACTCGAGAAAGAAGTG |
| | | GGCCGCTGCGAGAACATCGTGTCTAAGTACGAGACAAGA |
| | | TACGGGCCCCTGGGCAGCGGTAGCGGCTCC |
| E34-V (GS)3 | 82 | ITIRAAFLEKENTALRTEVAELEKEVGRCENIVSKYETRYGP |
| | (amino acid) | LGSGSGS |
| E34-N (GS)3 | 83 | ATTACAATCAGAGCCGCCTTCCTCGAGAAAGAGAACACA |
| | (nucleic acid) | GCCCTGAGAACCGAGAACGCCGAGCTGGAAAAAGAAGT |
| | | GGGCCGCTGCGAGAACATCGTGTCTAAGTACGAGACAAG |
| | | ATACGGCCCACTCGGCTCCGGATCTGGAAGC |
| E34-N (GS)3 | 84 | ITIRAAFLEKENTALRTENAELEKEVGRCENIVSKYETRYGP |
| | (amino acid) | LGSGSGS |
| CD94 (H)-rev | 85 | GGCGTTTGGAAGGAGCAGTGTTCGTGTTGTGACAGTGAC |
| | (nucleic acid) | AAGCAGCTGGAGATCAATCCGGGACCAACTTTTGCGCCT |
| | | GAAATTTCTCTGAAGACCTTCAGCAACAAG |
| CD94 (H)-rev | 86 | GVWKEQCSCCDSDKQLEINPGPTFAPEISLKTFSNK |
| | (amino acid) | |
| NKG2A (H)-rev | 87 | GGGTGCCATCGCGCCAAGCAGACGCGCACCAACCTCAGT |
| | (nucleic acid) | TCTAACAACCACCGCCAAATTCTAACAAGCCCC |
| NKG2A (H)-rev | 88 | GCHRAKQTRTNLSSNNHRQILTSP |
| | (amino acid) | |
| NKG2C (H)-rev | 89 | GGATGTCACCGCGCCAAACAGACTCGCACCAACCCCTCT |
| | (nucleic acid) | TCGAACAACCAGGAGCTGTTCCCCATC |
| NKG2C (H)-rev | 90 | GCHRAKQTRTNPSSNNQELFPI |
| | (amino acid) | |
| CD8 (SS) | 91 | ATGGCCCTGCCTGTGACAGCTCTGCTGCTGCCTCTGGCCC |
| | (nucleic acid) | TGCTGCTGCATGCTGCTAGACCT |
| CD8 (SS) | 92 | MALPVTALLLPLALLLHAARP |
| | (amino acid) | |
| aCEA (antigen | 93 | GACATCCAGCTGACACAGAGCCCTAGCAGCCTGTCTGCC |
| binding domain) | (nucleic acid) | TCTGTGGGCGACAGAGTGACCATCACATGCAAGGCCTCT |
| | | CAGGACGTGGGCACAAGCGTGGCATGGTATCAGCAGAAG |
| | | CCTGGCAAGGCCCCTAAGCTGCTGATCTACTGGACCAGC |
| | | ACCAGACACACAGGCGTGCCCAGCAGATTTTCTGGCAGC |
| | | GGCTCTGGCACCGACTTCACCTTCACCATAAGCAGCCTGC |
| | | AGCCTGAGGATATCGCCACCTACTACTGCCAGCAGTACA |
| | | GCCTGTACAGAAGCTTCGGCCAGGGCACCAAGGTGGAAA |
| | | TCAAAGGCGGATCTGGAAGCGGCGGTTCTGGATCTGGTG |
| | | GAAGCGGATCTGAGGTGCAGCTGGTGGAATCTGGTGGCG |
| | | GAGTTGTGCAGCCTGGCAGATCTCTGAGACTGAGCTGTA |
| | | GCGCCAGCGGCTTCGATTTCACCACCTACTGGATGAGCTG |
| | | GGTCCGACAGGCCCCTGGCAAAGGACTGGAATGGATCGG |
| | | CGAGATTCACCCCGACAGCAGCACCATCAATTACGCCCC |
| | | TAGCCTGAAGGACCGGTTCACCATCTCCAGAGACAACGC |
| | | CAAGAATACCCTGTTCCTGCAGATGGACAGCCTCCGGCCT |
| | | GAAGATACCGGCGTGTACTTTTGCGCCAGCCTGTATTTCG |
| | | GCTTCCCTTGGTTTGCCTACTGGGGCCAGGGAACACCTGT |
| | | GACCGTTAGCTCT |
| aCEA (antigen | 94 | DIQLTQSPSSLSASVGDRVTITCKASQDVGTSVAWYQQKPG |
| binding domain) | (amino acid) | KAPKLLIYWTSTRHTGVPSRFSGSGSGTDFTFTISSLQPEDIA |
| | | TYYCQQYSLYRSFGQGTKVEIKGGSGSGGSGSGGSGSEVQL |
| | | VESGGGVVQPGRSLRLSCSASGFDFTTYWMSWVRQAPGKG |
| | | LEWIGEIHPDSSTINYAPSLKDRFTISRDNAKNTLFLQMDSLR |
| | | PEDTGVYFCASLYFGFPWFAYWGQGTPVTVSS |
| aVSIG2 (antigen | 95 | GACATCCAGATGACACAGTCTCCAGCCAGCCTGTCTGCCT |
| binding domain) | (nucleic acid) | CTGTGGGAGAGACAGTGACCATGACCTGTCGGGCCAGCG |
| | | AGAACATCTACAGCTACCTGGCCTGGTATCAGCAGAAGC |
| | | AGGGCAAGTCTCCTCAGCTGCTGGTGTTCAACGCCGAGA |
| | | CACTGCCTGAAGGCGTGCCCAGCAGATTTTCTGGAACAG |
| | | GCAGCGGCACCCACTTCAGCCTGAGAATCAATAGCCTGC |
| | | AGCCTGAGGACTTCGGCAGCTACTACTGCCAGCACCACT |
| | | ACGTGATCCCTTGGACCTTTGGCGGAGGCACCAAGCTGG |
| | | AAATCAAGGGCAGCACAAGCGGCTCTGGAAAACCTGGAT |
| | | CTGGCGAGGGCTCTACCAAGGGCGAGGTGCAGATGGTTG |
| | | AGTCTGGCGGCGATCTGGTTAAGCCTGGCGGAAGCCTGA |
| | | AGCTGTCTTGTGCCGCCAGCGGCTTCACCTTCAGCAATAG |
| | | CGGCATGAGCTGGGTCCGACAGACCCCTGACAAGAGACT |
| | | GGAATGGGTCGCCAGCATCTCTGACGGCGGCCTGTACAC |
| | | ACACTACCCCGATTCTGTGAAGGGCAGATTCACCATCAG |
| | | CAGAGACAACGGCAAGAGCACCCTGTACCTGCAGATGAG |
| | | CAGCCTGAGAAGCGAGGACACCGCCATCTACTACTGCGC |
| | | CAGACAGGGCGTCAGACCCTTCTTCGATTATTGGGGCCA |
| | | GGGCACCACACTGACCGTGTCATCT |
| aVSIG2 (antigen | 96 | DIQMTQSPASLSASVGETVTMTCRASENIYSYLAWYQQKQG |
| binding domain) | (amino acid) | KSPQLLVFNAETLPEGVPSRFSGTGSGTHFSLRINSLQPEDFG |
| | | SYYCQHHYVIPWTFGGGTKLEIKGSTSGSGKPGSGEGSTKG |
| | | EVQMVESGGDLVKPGGSLKLSCAASGFTFSNSGMSWVRQT |
| | | PDKRLEWVASISDGGLYTHYPDSVKGRFTISRDNGKSTLYL |
| | | QMSSLRSEDTAIYYCARQGVRPFFDYWGQGTTLTVSS |
| myc | 97 | GAACAAAAACTCATCTCAGAAGAAGATCTGAATGGGGCC |
| | (nucleic acid) | GCA |
| myc | 98 | EQKLISEEDLNGAA |
| | (amino acid) | |
| V5 | 99 | GGGAAGCCTATCCCGAACCCTCTGTTGGGTCTCGATAGTA |
| | (nucleic acid) | CCAATGGGGCCGCA |
| V5 | 100 | GKPIPNPLLGLDSTNGAA |
| | (amino acid) | |
| CD8 (H) | 101 | ACCACGACGCCAGCGCCGCGACCACCAACACCGGCGCCC |
| | (nucleic acid) | ACCATCGCGTTGCAGCCCCTGTCCCTGCGCCCAGAGGCGT |
| | | GCCGGCCAGCGGCGGGGGGCGCAGTGCACACGAGGGGG |
| | | CTGGACTTCGCCTGTGAT |
| CD8 (H) | 102 | TTTPAPRPPTPAPTIALQPLSLRPEACRPAAGGAVHTRGLDF |
| | (amino acid) | ACD |
| CD28 (TM) | 103 | TTTTGGGTGCTGGTGGTGGTTGGTGGAGTCCTGGCTTGCT |
| | (nucleic acid) | ATAGCTTGCTAGTAACAGTGGCCTTTATTATTTTCTGGGT |
| | | G |
| CD28 (TM) | 104 | FWVLVVVGGVLACYSLLVTVAFIIFWV |
| | (amino acid) | |
| LIR1 (TM) | 105 | GTTATAGGGATCCTGGTGGCTGTCATACTCCTCTTGCTCC |
| | (nucleic acid) | TCTTGTTGCTGCTTTTTTTGATA |
| LIR1 (TM) | 106 | VIGILVAVILLLLLLLLLFLI |
| | (amino acid) | |
| CD28 (ICD) | 107 | AGGAGTAAGAGGAGCAGGCTCCTGCACAGTGACTACATG |
| | (nucleic acid) | AACATGACTCCCCGCCGCCCCGGGCCCACCCGCAAGCAT |
| | | TACCAGCCCTATGCCCCACCACGCGACTTCGCAGCCTATC |
| | | GCTCC |
| CD28 (ICD) | 108 | RSKRSRLLHSDYMNMTPRRPGPTRKHYQPYAPPRDFAAYRS |
| | (amino acid) | |
| LIR1 (ICD) | 109 | TTGCGCCACAGACGGCAGGGAAAGCACTGGACTAGTACG |
| | (nucleic acid) | CAGAGGAAAGCGGACTTCCAGCATCCCGCAGGAGCCGTG |
| | | GGGCCTGAACCCACTGATCGCGGCCTTCAATGGAGGTCT |
| | | AGCCCGGCGGCAGACGCACAAGAGGAAAACTTGTACGCA |
| | | GCCGTTAAGCACACCCAACCGGAGGACGGCGTTGAGATG |
| | | GATACCCGCTCCCCTCACGATGAAGACCCTCAAGCAGTC |
| | | ACTTACGCGGAAGTAAAGCATAGCCGCCCCAGACGGGAA |
| | | ATGGCTAGCCCGCCGTCCCCCCTTAGCGGGGAATTTCTGG |
| | | ACACTAAAGATAGGCAGGCGGAAGAGGACCGCCAAATG |
| | | GATACAGAGGCGGCGGCAAGTGAAGCACCTCAAGACGTT |
| | | ACTTACGCTCAACTTCACAGCCTTACCCTCAGGCGAGAAG |
| | | CGACTGAACCACCCCCTTCCCAAGAAGGGCCAAGCCCAG |
| | | CGGTTCCTTCTATCTATGCTACTCTTGCTATTCAC |
| LIR1 (ICD) | 110 | LRHRRQGKHWTSTQRKADFQHPAGAVGPEPTDRGLQWRSS |
| | (amino acid) | PAADAQEENLYAAVKHTQPEDGVEMDTRSPHDEDPQAVTY |
| | | AEVKHSRPRREMASPPSPLSGEFLDTKDRQAEEDRQMDTEA |
| | | AASEAPQDVTYAQLHSLTLRREATEPPPSQEGPSPAVPSIYA |
| | | TLAIH |
| Z (stim/zipper) | 111 | AGAGTGAAGTTCAGCAGGAGCGCAGACGCCCCCGCGTAC |
| | (nucleic acid) | AAGCAGGGCCAGAACCAGCTCTATAACGAGCTCAATCTA |
| | | GGACGAAGAGAGGAGTACGATGTTTTGGACAAGAGACGT |
| | | GGCCGGGACCCTGAGATGGGGGGAAAGCCGAGAAGGAA |
| | | GAACCCTCAGGAAGGCCTGTACAATGAACTGCAGAAAGA |
| | | TAAGATGGCGGAGGCCTACAGTGAGATTGGGATGAAAGG |
| | | CGAGCGCCGGAGGGGCAAGGGGCACGATGGCCTTTACCA |
| | | GGGTCTCAGTACAGCCACCAAGGACACCTACGACGCCCT |
| | | TCACATGCAGGCCCTGCCCCCTCGC |
| Z (stim/zipper) | 112 | RVKFSRSADAPAYKQGQNQLYNELNLGRREEYDVLDKRRG |
| | (amino acid) | RDPEMGGKPRRKNPQEGLYNELQKDKMAEAYSEIGMKGER |
| | | RRGKGHDGLYQGLSTATKDTYDALHMQALPPR |
| GS(3) R34 | 113 | GGATCAGGATCTGGCAGCCTGGAAATCAGGGCCGCCTTC |
| (stim/zipper) | (nucleic acid) | CTGGAAAAAGAGAACACAGCCCTGCGGACAAGAGCCGC |
| | | CGAGCTGAGAAAACGCGTGGGCAGATGCCGGAACATCGT |
| | | GTCTAAGTACGAGACAAGATACGGCCCTCTG |
| GS(3) R34 | 114 | GSGSGSLEIRAAFLEKENTALRTRAAELRKRVGRCRNIVSKY |
| (stim/zipper) | (amino acid) | ETRYGPL |
| GS(3) E34-I | 115 | GGAAGCGGATCCGGCAGCATTACAATTAGAGCCGCATTC |
| (stim/zipper) | (nucleic acid) | CTCGAGAAAGAAAACACCGCTCTGAGAACCGAGATTGCC |
| | | GAACTCGAAAAAGAAGTGGGGCGCTGCGAGAATATCGTG |
| | | TCCAAATACGAAACCCGCTACGGCCCACTC |
| GS(3) E34-I | 116 | GSGSGSITIRAAFLEKENTALRTEIAELEKEVGRCENIVSKYE |
| (stim/zipper) | (amino acid) | TRYGPL |
| GS(3) E34-V | 117 | GGTAGCGGCTCCGGCTCCATTACAATTAGAGCCGCCTTCC |
| (stim/zipper) | (nucleic acid) | TGGAAAAAGAGAACACAGCCCTGAGAACCGAGGTGGCC |
| | | GAACTCGAGAAAGAAGTGGGCCGCTGCGAGAACATCGTG |
| | | TCTAAGTACGAGACAAGATACGGGCCCCTG |
| GS(3) E34-V | 118 | GSGSGSITIRAAFLEKENTALRTEVAELEKEVGRCENIVSKY |
| (stim/zipper) | (amino acid) | ETRYGPL |
| GS(3) E34-N | 119 | GGATCTGGCTCTGGATCCATTACAATCAGAGCCGCCTTCC |
| (stim/zipper) | (nucleic acid) | TCGAGAAAGAGAACACAGCCCTGAGAACCGAGAACGCC |
| | | GAGCTGGAAAAAGAAGTGGGCCGCTGCGAGAACATCGTG |
| | | TCTAAGTACGAGACAAGATACGGCCCACTC |
| GS(3) E34-N | 120 | GSGSGSITIRAAFLEKENTALRTENAELEKEVGRCENIVSKY |
| (stim/zipper) | (amino acid) | ETRYGPL |
| P2A PuroR | 121 | GGAAGCGGAGCTACTAACTTCAGCCTGCTGAAGCAGGCT |
| (selection) | (nucleic acid) | GGAGACGTGGAGGAGAACCCTGGACCTATGACCGAGTAC |
| | | AAGCCCACGGTGCGCCTCGCCACCCGCGACGACGTCCCC |
| | | AGGGCCGTACGCACCCTCGCCGCCGCGTTCGCCGACTAC |
| | | CCCGCCACGCGCCACACCGTCGATCCGGACCGCCACATC |
| | | GAGCGGGTCACCGAGCTGCAAGAACTCTTCCTCACGCGC |
| | | GTCGGGCTCGACATCGGCAAGGTGTGGGTCGCGGACGAC |
| | | GGCGCCGCGGTGGCGGTCTGGACCACGCCGGAGAGCGTC |
| | | GAAGCGGGGGCGGTGTTCGCCGAGATCGGCCCGCGCATG |
| | | GCCGAGTTGAGCGGTTCCCGGCTGGCCGCGCAGCAACAG |
| | | ATGGAAGGCCTCCTGGCGCCGCACCGGCCCAAGGAGCCC |
| | | GCGTGGTTCCTGGCCACCGTCGGCGTCTCGCCCGACCACC |
| | | AGGGCAAGGGTCTGGGCAGCGCCGTCGTGCTCCCCGGAG |
| | | TGGAGGCGGCCGAGCGCGCCGGGGTGCCCGCCTTCCTGG |
| | | AGACCTCCGCGCCCCGCAACCTCCCCTTCTACGAGCGGCT |
| | | CGGCTTCACCGTCACCGCCGACGTCGAGGTGCCCGAAGG |
| | | ACCGCGCACCTGGTGCATGACCCGCAAGCCCGGTGCC |
| P2A PuroR | 122 | GSGATNFSLLKQAGDVEENPGPMTEYKPTVRLATRDDVPR |
| (selection) | (amino acid) | AVRTLAAAFADYPATRHTVDPDRHIERVTELQELFLTRVGL |
| | | DIGKVWVADDGAAVAVWTTPESVEAGAVFAEIGPRMAELS |
| | | GSRLAAQQQMEGLLAPHRPKEPAWFLATVGVSPDHQGKGL |
| | | GSAVVLPGVEAAERAGVPAFLETSAPRNLPFYERLGFTVTA |
| | | DVEVPEGPRTWCMTRKPGA |

What is claimed is:

1. A chimeric protein system comprising:
a first polypeptide comprising a first antigen binding domain, a first transmembrane domain, and a first dimerization domain; and a second polypeptide comprising a second antigen binding domain, a second transmembrane domain, and a second dimerization domain; wherein the first dimerization domain is capable of binding to the second dimerization domain, optionally wherein the first dimerization domain or the second dimerization domain is selected from the group consisting of CD94, NKG2A, and NKG2C.

2. The chimeric protein system of claim 1 or 2, wherein the first dimerization domain comprises a dimerization domain from CD94, and the second dimerization domain comprises a dimerization domain from NKG2A or a dimerization domain from NKG2C, optionally wherein the second dimerization domain comprises a sequence selected from the group consisting of SEQ ID NO: 70, 72, 74, 76, 78, 80, 82, 84, 86, 88, and 90.

3. The chimeric protein system of claim 1 or 2, wherein the first dimerization domain comprises a dimerization domain from NKG2A or a dimerization domain from NKG2C, and the second dimerization domain comprises a dimerization domain from CD94, optionally wherein the first dimerization domain comprises a sequence selected from the group consisting of SEQ ID NOs: 70, 72, 74, 76, 78, 80, 82, 84, 86, 88, and 90.

4. The chimeric protein system of any one of claims 1 to 6, wherein the first polypeptide is a chimeric antigen receptor, optionally wherein the first polypeptide is an activating chimeric antigen receptor or an inhibitory chimeric antigen receptor.

5. The chimeric protein system of any one of claims 1 to 9, wherein the second polypeptide is a chimeric antigen receptor, optionally wherein the second polypeptide is an activating chimeric antigen receptor or an inhibitory chimeric antigen receptor.

6. The chimeric protein system of any one of claims 1 to 12, wherein the antigen binding domain comprises a F(ab) fragment, a F(ab′) fragment, a single chain variable fragment (scFv), a single domain antibody, a diabody, a VHH fragment, or a synthetic epitope.

7. The chimeric protein system of claim 13, wherein the antigen binding domain binds an antigen expressed on a cancer cell, optionally wherein the cancer comprises glioblastoma, neuroblastoma, breast cancer, colorectal cancer, prostate cancer, bladder cancer, liver cancer, lung cancer, pancreatic cancer, ovarian cancer, gastric cancer, endometrial cancer, cervical cancer, leukemia, lymphoma, or myeloma, optionally wherein the antigen binding domain is specific for carcinoembryonic antigen (CEA), Mesothelin, Ax1, GPC3, FLT3, CD33, TROP2, MUC1, MUC16, IL13Ra, ErbB2 (HER2/neu), epithelial cell adhesion molecule (EpCAM), epidermal growth factor receptor (EGFR), EGFR variant III (EGFRvIII), CD19, CD20, CD30, CD40, disialoganglioside GD2, ductal-epithelial mucine, gp36, TAG-72, glycosphingolipids, glioma-associated antigen, alphafetoprotein (AFP), lectin-reactive AFP, thyroglobulin, RAGE 1, MN-CA IX, human telomerase reverse transcriptase, RU1, RU2 (AS), intestinal carboxyl esterase, mut hsp70-2, M-CSF, prostate specific antigen (PSA), PAP, NY-ESO-1, LAGA-1a, p53, prostein, PSMA, prostate-carcinoma tumor antigen-1 (PCTA-1), MAGE, ELF2M, neutrophil elastase, ephrin B2, CD22, insulin growth factor (IGF)-1, IGF-11, IGF-1 receptor, NKG2D, BCMA (CD269, TNFRSF 17), Claudin18.2, B7-H3, or Ror1.

8. The chimeric protein system of any one of claims 1 to 16, wherein the transmembrane domain is selected from the group consisting of: a LAX transmembrane domain, a CD25 transmembrane domain, a CD7 transmembrane domain, a LAT transmembrane domain, a transmembrane domain from a LAT mutant, a BTLA transmembrane domain, a CDS transmembrane domain, a CD28 transmembrane domain, a CD3zeta transmembrane domain, a CD4 transmembrane domain, a 4-IBB transmembrane domain, an OX40 transmembrane domain, an ICOS transmembrane domain, a 2B4 transmembrane domain, a PD-1 transmembrane domain, a CTLA4 transmembrane domain, a BTLA transmembrane domain, a TIM3 transmembrane domain, a LIR1 transmembrane domain, an NKG2A transmembrane domain, a TIGIT transmembrane domain, and a LAG3 transmembrane domain, a LAIR1 transmembrane domain, a GRB-2 transmembrane domain, a Dok-1 transmembrane domain, a Dok-2 transmembrane domain, a SLAP1 transmembrane domain, a SLAP2 transmembrane domain, a CD200R transmembrane domain, an SIRPa transmembrane domain, an HAVR transmembrane domain, a GITR transmembrane domain, a PD-L1 transmembrane domain, a KIR2DL1 transmembrane domain, a KIR2DL2 transmembrane domain, a KIR2DL3 transmembrane domain, a KIR3DL1 transmembrane domain, a KIR3DL2 transmembrane domain, a CD94 transmembrane domain, a KLRG-1 transmembrane domain, a PAG transmembrane domain, a CD45 transmembrane domain, and a CEACAM1 transmembrane domain.

9. The chimeric protein system of any one of claims 1 to 17, wherein the first polypeptide and/or the second polypeptide further comprises one or more intracellular signaling domains, optionally wherein the one or more intracellular signaling domains are selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CDS intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain, optionally wherein the one or more intracellular signaling domains comprises a co-stimulatory domain selected from the group consisting of: a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CDS intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.

10. The chimeric protein system of any one of claims 1 to 20, wherein the first polypeptide and/or the second polypeptide comprises a hinge domain located between the antigen binding domain and the transmembrane domain.

11. The chimeric protein system of any one of claims 1 to 21, wherein the first polypeptide and/or the second polypeptide comprises one or more linkers, optionally wherein the one or more linkers comprise a GSG linker, a Whitlow linker, an eGK linker, or any derivative thereof.

12. An engineered polynucleotide encoding the first polynucleotide and/or the second polynucleotide of any one of claims 1 to 23.

13. An expression vector comprising the engineered polynucleotide of claim 24.

14. An engineered cell comprising the engineered polynucleotide of claim 25, the vector of claim 25, or the first and/or second polypeptides of any one of claims 1 to 24.

15. The engineered cell of claim 26, wherein the cell is selected from the group consisting of: a T cell, a CDS+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell, optionally wherein the cell is engineered to express an effector molecule, optionally wherein the engineered cell of claim 28, wherein the cell is a Natural Killer (NK) cell, optionally wherein the cell is allogeneic or autologous.

16. A pharmaceutical composition comprising the engineered cell of any one of claims 26 to 31, and a pharmaceutically acceptable carrier and/or a pharmaceutically acceptable excipient.

17. A method of treating a subject having cancer, the method comprising administering to the subject a therapeutically effective dose of the composition of claim 32, or a cell of any one of claims 26 to 31 to the subject, optionally wherein the cancer comprises glioblastoma, neuroblastoma, breast cancer, colorectal cancer, prostate cancer, bladder cancer, liver cancer, lung cancer, pancreatic cancer, ovarian cancer, gastric cancer, endometrial cancer, cervical cancer, leukemia, lymphoma, or myeloma.

18. A method of enhancing immune cell-mediated killing of a cancer cell in a subject in need thereof, the method comprising administering a therapeutically effective dose of the composition of claim 32, or a cell of any one of claims 26 to 31, to the subject, optionally wherein the cancer comprises a solid tumor, optionally wherein the cancer comprises glioblastoma, neuroblastoma, breast cancer, colorectal cancer, prostate cancer, bladder cancer, liver cancer, lung cancer, pancreatic cancer, ovarian cancer, gastric cancer, endometrial cancer, cervical cancer, leukemia, lymphoma, or myeloma.

19. A method of reducing off-target killing of a healthy cell in a subject, the method comprising administering a therapeutically effective dose of the composition of claim 32, or a cell of any one of claims 26 to 31, to the subject, wherein the subject has been diagnosed with cancer, optionally wherein the cancer comprises a solid tumor, optionally wherein the cancer comprises glioblastoma, neuroblastoma, breast cancer, colorectal cancer, prostate cancer, bladder cancer, liver cancer, lung cancer, pancreatic cancer, ovarian cancer, gastric cancer, endometrial cancer, cervical cancer, leukemia, lymphoma, or myeloma.


SEQUENCE LISTING (182 sequences)

SEQ ID NO: 1 - DNA, 1473 bp, Synthetic construct
  source 1..1473: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaaagaacagcttcaccaagctgagcatcgagcccgccttcacacccggacctaatatcgagctgcagaaggacagcgactgctgcagctgccaagagaaatgggtcggattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 2 - protein, 491 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAAKNSFTKLSIEPAFTPGPNIELQKDSDCCSCQEKWVGFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 1518 bp, Synthetic construct
  source 1..1518: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaaagaacagcttcaccaagctgagcatcgagcccgccttcacacccggacctaatatcgagctgcagaaggacagcgactgctgcagctgccaagagaaatgggtcggataccggtgcaactgctacttcatcagcagcgagcagaaaacctggttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 4 - protein, 506 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAAKNSFTKLSIEPAFTPGPNIELQKDSDCCSCQEKWVGYRCNCYFISSEQKTWFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 1437 bp, Synthetic construct
  source 1..1437: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcacccagcacactgatccagcggcacaacaacagcagcctgaacaccagaacacagaaggcccggcactgcggcttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 6 - protein, 479 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAAPSTLIQRHNNSSLNTRTQKARHCGFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - DNA, 1431 bp, Synthetic construct
  source 1..1431: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaatcccattcctcgagcagaacaacagcagccccaacacaagaacccagaaggccagacactgcggattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 8 - protein, 477 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAAIPFLEQNNSSPNTRTQKARHCGFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 1572 bp, Synthetic construct
  source 1..1572: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcacccagcacactgatccagcggcacaacaacagcagcctgaacaccagaacacagaaggcccggcactgcggcaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 10 - protein, 524 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAAPSTLIQRHNNSSLNTRTQKARHCGTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1566 bp, Synthetic construct
  source 1..1566: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaatcccattcctcgagcagaacaacagcagccccaacacaagaacccagaaggccagacactgcggaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 12 - protein, 522 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAAIPFLEQNNSSPNTRTQKARHCGTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 2184 bp, Synthetic construct
  source 1..2184: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaaagaacagcttcaccaagctgagcatcgagcccgccttcacacccggacctaatatcgagctgcagaaggacagcgactgctgcagctgccaagagaaatgggtcggagttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 14 - protein, 728 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAAKNSFTKLSIEPAFTPGPNIELQKDSDCCSCQEKWVGVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 15 - DNA, 2229 bp, Synthetic construct
  source 1..2229: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaaagaacagcttcaccaagctgagcatcgagcccgccttcacacccggacctaatatcgagctgcagaaggacagcgactgctgcagctgccaagagaaatgggtcggataccggtgcaactgctacttcatcagcagcgagcagaaaacctgggttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 16 - protein, 743 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAAKNSFTKLSIEPAFTPGPNIELQKDSDCCSCQEKWVGYRCNCYFISSEQKTWVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 17 - DNA, 2148 bp, Synthetic construct
  source 1..2148: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcacccagcacactgatccagcggcacaacaacagcagcctgaacaccagaacacagaaggcccggcactgcggcgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 18 - protein, 716 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAAPSTLIQRHNNSSLNTRTQKARHCGVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 19 - DNA, 2142 bp, Synthetic construct
  source 1..2142: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaatcccattcctcgagcagaacaacagcagccccaacacaagaacccagaaggccagacactgcggagttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 20 - protein, 714 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAAIPFLEQNNSSPNTRTQKARHCGVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 21 - DNA, 2283 bp, Synthetic construct
  source 1..2283: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcacccagcacactgatccagcggcacaacaacagcagcctgaacaccagaacacagaaggcccggcactgcggcaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 22 - protein, 761 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAAPSTLIQRHNNSSLNTRTQKARHCGTTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 23 - DNA, 2277 bp, Synthetic construct
  source 1..2277: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaatcccattcctcgagcagaacaacagcagccccaacacaagaacccagaaggccagacactgcggaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 24 - protein, 759 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAAIPFLEQNNSSPNTRTQKARHCGTTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 25 - DNA, 1647 bp, Synthetic construct
  source 1..1647: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcactggaaatcagggccgccttcctggaaaaagagaacacagccctgcggacaagagccgccgagctgagaaaacgcgtgggcagatgccggaacatcgtgtctaagtacgagacaagatacggccctctgggctctggtagtggatctaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 26 - protein, 549 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAALEIRAAFLEKENTALRTRAAELRKRVGRCRNIVSKYETRYGPLGSGSGSTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1647 bp, Synthetic construct
  source 1..1647: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaattacaattagagccgcattcctcgagaaagaaaacaccgctctgagaaccgagattgccgaactcgaaaaagaagtggggcgctgcgagaatatcgtgtccaaatacgaaacccgctacggcccactcggctctggtagtggatctaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 28 - protein, 549 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAAITIRAAFLEKENTALRTEIAELEKEVGRCENIVSKYETRYGPLGSGSGSTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 1647 bp, Synthetic construct
  source 1..1647: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaattacaattagagccgccttcctggaaaaagagaacacagccctgagaaccgaggtggccgaactcgagaaagaagtgggccgctgcgagaacatcgtgtctaagtacgagacaagatacgggcccctgggcagcggtagcggctccaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 30 - protein, 549 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAAITIRAAFLEKENTALRTEVAELEKEVGRCENIVSKYETRYGPLGSGSGSTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 1647 bp, Synthetic construct
  source 1..1647: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaattacaatcagagccgccttcctcgagaaagagaacacagccctgagaaccgagaacgccgagctggaaaaagaagtgggccgctgcgagaacatcgtgtctaagtacgagacaagatacggcccactcggctccggatctggaagcaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 32 - protein, 549 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAAITIRAAFLEKENTALRTENAELEKEVGRCENIVSKYETRYGPLGSGSGSTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 2358 bp, Synthetic construct
  source 1..2358: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcactggaaatcagggccgccttcctggaaaaagagaacacagccctgcggacaagagccgccgagctgagaaaacgcgtgggcagatgccggaacatcgtgtctaagtacgagacaagatacggccctctgggctctggtagtggatctaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 34 - protein, 786 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAALEIRAAFLEKENTALRTRAAELRKRVGRCRNIVSKYETRYGPLGSGSGSTTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 35 - DNA, 2358 bp, Synthetic construct
  source 1..2358: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaattacaattagagccgcattcctcgagaaagaaaacaccgctctgagaaccgagattgccgaactcgaaaaagaagtggggcgctgcgagaatatcgtgtccaaatacgaaacccgctacggcccactcggctctggtagtggatctaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 36 - protein, 786 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAAITIRAAFLEKENTALRTEIAELEKEVGRCENIVSKYETRYGPLGSGSGSTTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 37 - DNA, 2358 bp, Synthetic construct
  source 1..2358: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaattacaattagagccgccttcctggaaaaagagaacacagccctgagaaccgaggtggccgaactcgagaaagaagtgggccgctgcgagaacatcgtgtctaagtacgagacaagatacgggcccctgggcagcggtagcggctccaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 38 - protein, 786 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAAITIRAAFLEKENTALRTEVAELEKEVGRCENIVSKYETRYGPLGSGSGSTTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 39 - DNA, 2358 bp, Synthetic construct
  source 1..2358: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaattacaatcagagccgccttcctcgagaaagagaacacagccctgagaaccgagaacgccgagctggaaaaagaagtgggccgctgcgagaacatcgtgtctaagtacgagacaagatacggcccactcggctccggatctggaagcaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 40 - protein, 786 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAAITIRAAFLEKENTALRTENAELEKEVGRCENIVSKYETRYGPLGSGSGSTTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 41 - DNA, 1647 bp, Synthetic construct
  source 1..1647: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatcaggatctggcagcctggaaatcagggccgccttcctggaaaaagagaacacagccctgcggacaagagccgccgagctgagaaaacgcgtgggcagatgccggaacatcgtgtctaagtacgagacaagatacggccctctg

SEQ ID NO: 42 - protein, 549 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGSGSLEIRAAFLEKENTALRTRAAELRKRVGRCRNIVSKYETRYGPL

SEQ ID NO: 43 - DNA, 1647 bp, Synthetic construct
  source 1..1647: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggatccggcagcattacaattagagccgcattcctcgagaaagaaaacaccgctctgagaaccgagattgccgaactcgaaaaagaagtggggcgctgcgagaatatcgtgtccaaatacgaaacccgctacggcccactc

SEQ ID NO: 44 - protein, 549 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGSGSITIRAAFLEKENTALRTEIAELEKEVGRCENIVSKYETRYGPL

SEQ ID NO: 45 - DNA, 1647 bp, Synthetic construct
  source 1..1647: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggctccggctccattacaattagagccgccttcctggaaaaagagaacacagccctgagaaccgaggtggccgaactcgagaaagaagtgggccgctgcgagaacatcgtgtctaagtacgagacaagatacgggcccctg

SEQ ID NO: 46 - protein, 549 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGSGSITIRAAFLEKENTALRTEVAELEKEVGRCENIVSKYETRYGPL

SEQ ID NO: 47 - DNA, 1647 bp, Synthetic construct
  source 1..1647: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatctggctctggatccattacaatcagagccgccttcctcgagaaagagaacacagccctgagaaccgagaacgccgagctggaaaaagaagtgggccgctgcgagaacatcgtgtctaagtacgagacaagatacggcccactc

SEQ ID NO: 48 - protein, 549 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGSGSITIRAAFLEKENTALRTENAELEKEVGRCENIVSKYETRYGPL

SEQ ID NO: 49 - DNA, 2358 bp, Synthetic construct
  source 1..2358: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggatcaggatctggcagcctggaaatcagggccgccttcctggaaaaagagaacacagccctgcggacaagagccgccgagctgagaaaacgcgtgggcagatgccggaacatcgtgtctaagtacgagacaagatacggccctctgggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 50 - protein, 786 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAATTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGSGSLEIRAAFLEKENTALRTRAAELRKRVGRCRNIVSKYETRYGPLGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 51 - DNA, 2358 bp, Synthetic construct
  source 1..2358: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggatccggcagcattacaattagagccgcattcctcgagaaagaaaacaccgctctgagaaccgagattgccgaactcgaaaaagaagtggggcgctgcgagaatatcgtgtccaaatacgaaacccgctacggcccactcggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 52 - protein, 786 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAATTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGSGSITIRAAFLEKENTALRTEIAELEKEVGRCENIVSKYETRYGPLGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 53 - DNA, 2358 bp, Synthetic construct
  source 1..2358: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggtagcggctccggctccattacaattagagccgccttcctggaaaaagagaacacagccctgagaaccgaggtggccgaactcgagaaagaagtgggccgctgcgagaacatcgtgtctaagtacgagacaagatacgggcccctgggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 54 - protein, 786 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAATTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGSGSITIRAAFLEKENTALRTEVAELEKEVGRCENIVSKYETRYGPLGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 55 - DNA, 2358 bp, Synthetic construct
  source 1..2358: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggatctggctctggatccattacaatcagagccgccttcctcgagaaagagaacacagccctgagaaccgagaacgccgagctggaaaaagaagtgggccgctgcgagaacatcgtgtctaagtacgagacaagatacggcccactcggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 56 - protein, 786 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAATTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGSGSITIRAAFLEKENTALRTENAELEKEVGRCENIVSKYETRYGPLGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 57 - DNA, 1473 bp, Synthetic construct
  source 1..1473: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaggcgtttggaaggagcagtgttcgtgttgtgacagtgacaagcagctggagatcaatccgggaccaacttttgcgcctgaaatttctctgaagaccttcagcaacaagttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 58 - protein, 491 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAAGVWKEQCSCCDSDKQLEINPGPTFAPEISLKTFSNKFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - DNA, 1437 bp, Synthetic construct
  source 1..1437: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcagggtgccatcgcgccaagcagacgcgcaccaacctcagttctaacaaccaccgccaaattctaacaagccccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 60 - protein, 479 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAAGCHRAKQTRTNLSSNNHRQILTSPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - DNA, 1431 bp, Synthetic construct
  source 1..1431: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaggatgtcaccgcgccaaacagactcgcaccaacccctcttcgaacaaccaggagctgttccccatcttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 62 - protein, 477 aa, Synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAAGCHRAKQTRTNPSSNNQELFPIFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - DNA, 2184 bp, Synthetic construct
  source 1..2184: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaggcgtttggaaggagcagtgttcgtgttgtgacagtgacaagcagctggagatcaatccgggaccaacttttgcgcctgaaatttctctgaagaccttcagcaacaaggttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 64 - protein, 728 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAAGVWKEQCSCCDSDKQLEINPGPTFAPEISLKTFSNKVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 65 - DNA, 2148 bp, Synthetic construct
  source 1..2148: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcagggtgccatcgcgccaagcagacgcgcaccaacctcagttctaacaaccaccgccaaattctaacaagccccgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 66 - protein, 716 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAAGCHRAKQTRTNLSSNNHRQILTSPVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 67 - DNA, 2142 bp, Synthetic construct
  source 1..2142: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaggatgtcaccgcgccaaacagactcgcaccaacccctcttcgaacaaccaggagctgttccccatcgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcacggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 68 - protein, 714 aa, Synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAAGCHRAKQTRTNPSSNNQELFPIVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 69 - DNA, 108 bp, Synthetic construct
  source 1..108: mol_type = other DNA
aagaacagcttcaccaagctgagcatcgagcccgccttcacacccggacctaatatcgagctgcagaaggacagcgactgctgcagctgccaagagaaatgggtcgga

SEQ ID NO: 70 - protein, 36 aa, Synthetic construct
KNSFTKLSIEPAFTPGPNIELQKDSDCCSCQEKWVG

SEQ ID NO: 71 - DNA, 153 bp, Synthetic construct
  source 1..153: mol_type = other DNA
aagaacagcttcaccaagctgagcatcgagcccgccttcacacccggacctaatatcgagctgcagaaggacagcgactgctgcagctgccaagagaaatgggtcggataccggtgcaactgctacttcatcagcagcgagcagaaaacctgg

SEQ ID NO: 72 - protein, 51 aa, Synthetic construct
KNSFTKLSIEPAFTPGPNIELQKDSDCCSCQEKWVGYRCNCYFISSEQKTW

SEQ ID NO: 73 - DNA, 72 bp, Synthetic construct
  source 1..72: mol_type = other DNA
cccagcacactgatccagcggcacaacaacagcagcctgaacaccagaacacagaaggcccggcactgcggc

SEQ ID NO: 74 - protein, 24 aa, Synthetic construct
PSTLIQRHNNSSLNTRTQKARHCG

SEQ ID NO: 75 - DNA, 66 bp, Synthetic construct
  source 1..66: mol_type = other DNA
atcccattcctcgagcagaacaacagcagccccaacacaagaacccagaaggccagacactgcgga

SEQ ID NO: 76 - protein, 22 aa, Synthetic construct
IPFLEQNNSSPNTRTQKARHCG

SEQ ID NO: 77 - DNA, 147 bp, Synthetic construct
  source 1..147: mol_type = other DNA
ctggaaatcagggccgccttcctggaaaaagagaacacagccctgcggacaagagccgccgagctgagaaaacgcgtgggcagatgccggaacatcgtgtctaagtacgagacaagatacggccctctgggctctggtagtggatct

SEQ ID NO: 78 - protein, 49 aa, Synthetic construct
LEIRAAFLEKENTALRTRAAELRKRVGRCRNIVSKYETRYGPLGSGSGS

SEQ ID NO: 79 - DNA, 147 bp, Synthetic construct
  source 1..147: mol_type = other DNA
attacaattagagccgcattcctcgagaaagaaaacaccgctctgagaaccgagattgccgaactcgaaaaagaagtggggcgctgcgagaatatcgtgtccaaatacgaaacccgctacggcccactcggctctggtagtggatct

SEQ ID NO: 80 - protein, 49 aa, Synthetic construct
ITIRAAFLEKENTALRTEIAELEKEVGRCENIVSKYETRYGPLGSGSGS

SEQ ID NO: 81 - DNA, 147 bp, Synthetic construct
  source 1..147: mol_type = other DNA
attacaattagagccgccttcctggaaaaagagaacacagccctgagaaccgaggtggccgaactcgagaaagaagtgggccgctgcgagaacatcgtgtctaagtacgagacaagatacgggcccctgggcagcggtagcggctcc

SEQ ID NO: 82 - protein, 49 aa, Synthetic construct
ITIRAAFLEKENTALRTEVAELEKEVGRCENIVSKYETRYGPLGSGSGS

SEQ ID NO: 83 - DNA, 147 bp, Synthetic construct
  source 1..147: mol_type = other DNA
attacaatcagagccgccttcctcgagaaagagaacacagccctgagaaccgagaacgccgagctggaaaaagaagtgggccgctgcgagaacatcgtgtctaagtacgagacaagatacggcccactcggctccggatctggaagc

SEQ ID NO: 84 - protein, 49 aa, Synthetic construct
ITIRAAFLEKENTALRTENAELEKEVGRCENIVSKYETRYGPLGSGSGS

SEQ ID NO: 85 - DNA, 108 bp, Synthetic construct
  source 1..108: mol_type = other DNA
ggcgtttggaaggagcagtgttcgtgttgtgacagtgacaagcagctggagatcaatccgggaccaacttttgcgcctgaaatttctctgaagaccttcagcaacaag

SEQ ID NO: 86 - protein, 36 aa, Synthetic construct
GVWKEQCSCCDSDKQLEINPGPTFAPEISLKTFSNK

SEQ ID NO: 87 - DNA, 72 bp, Synthetic construct
  source 1..72: mol_type = other DNA
gggtgccatcgcgccaagcagacgcgcaccaacctcagttctaacaaccaccgccaaattctaacaagcccc

SEQ ID NO: 88 - protein, 24 aa, Synthetic construct
GCHRAKQTRTNLSSNNHRQILTSP

SEQ ID NO: 89 - DNA, 66 bp, Synthetic construct
  source 1..66: mol_type = other DNA
ggatgtcaccgcgccaaacagactcgcaccaacccctcttcgaacaaccaggagctgttccccatc

SEQ ID NO: 90 - protein, 22 aa, Synthetic construct
GCHRAKQTRTNPSSNNQELFPI

SEQ ID NO: 91 - DNA, 63 bp, Synthetic construct
  source 1..63: mol_type = other DNA
atggccctgcctgtgacagctctgctgctgcctctggccctgctgctgcatgctgctagacct

SEQ ID NO: 92 - protein, 21 aa, Synthetic construct
MALPVTALLLPLALLLHAARP

SEQ ID NO: 93 - DNA, 720 bp, Synthetic construct
  source 1..720: mol_type = other DNA
gacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctct

SEQ ID NO: 94 - protein, 240 aa, Synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 95 - DNA, 729 bp, Synthetic construct
  source 1..729: mol_type = other DNA
gacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatct

SEQ ID NO: 96 - protein, 243 aa, Synthetic construct
DIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSS

SEQ ID NO: 97 - DNA, 42 bp, Synthetic construct
  source 1..42: mol_type = other DNA
gaacaaaaactcatctcagaagaagatctgaatggggccgca

SEQ ID NO: 98 - protein, 14 aa, Synthetic construct
EQKLISEEDLNGAA

SEQ ID NO: 99 - DNA, 54 bp, Synthetic construct
  source 1..54: mol_type = other DNA
gggaagcctatcccgaaccctctgttgggtctcgatagtaccaatggggccgca

SEQ ID NO: 100 - protein, 18 aa, Synthetic construct
GKPIPNPLLGLDSTNGAA

SEQ ID NO: 101 - DNA, 135 bp, Synthetic construct
  source 1..135: mol_type = other DNA
accacgacgccagcgccgcgaccaccaacaccggcgcccaccatcgcgttgcagcccctgtccctgcgcccagaggcgtgccggccagcggcggggggcgcagtgcacacgagggggctggacttcgcctgtgat

SEQ ID NO: 102 - protein, 45 aa, Synthetic construct
TTTPAPRPPTPAPTIALQPLSLRPEACRPAAGGAVHTRGLDFACD

SEQ ID NO: 103 - DNA, 81 bp, Synthetic construct
  source 1..81: mol_type = other DNA
ttttgggtgctggtggtggttggtggagtcctggcttgctatagcttgctagtaacagtggcctttattattttctgggtg

SEQ ID NO: 104 - protein, 27 aa, Synthetic construct
FWVLVVVGGVLACYSLLVTVAFIIFWV

SEQ ID NO: 105 - DNA, 63 bp, Synthetic construct
  source 1..63: mol_type = other DNA
gttatagggatcctggtggctgtcatactcctcttgctcctcttgttgctgctttttttgata

SEQ ID NO: 106 - protein, 21 aa, Synthetic construct
VIGILVAVILLLLLLLLLFLI

SEQ ID NO: 107 - DNA, 123 bp, Synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 108 - protein, 41 aa, Synthetic construct
RSKRSRLLHSDYMNMTPRRPGPTRKHYQPYAPPRDFAAYRS

SEQ ID NO: 109 - DNA, 504 bp, Synthetic construct
  source 1..504: mol_type = other DNA
ttgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcac

SEQ ID NO: 110 - protein, 168 aa, Synthetic construct
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 111 - DNA, 336 bp, Synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 112 - protein, 112 aa, Synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - DNA, 147 bp, Synthetic construct
  source 1..147: mol_type = other DNA
ggatcaggatctggcagcctggaaatcagggccgccttcctggaaaaagagaacacagccctgcggacaagagccgccgagctgagaaaacgcgtgggcagatgccggaacatcgtgtctaagtacgagacaagatacggccctctg

SEQ ID NO: 114 - protein, 49 aa, Synthetic construct
GSGSGSLEIRAAFLEKENTALRTRAAELRKRVGRCRNIVSKYETRYGPL

SEQ ID NO: 115 - DNA, 147 bp, Synthetic construct
  source 1..147: mol_type = other DNA
ggaagcggatccggcagcattacaattagagccgcattcctcgagaaagaaaacaccgctctgagaaccgagattgccgaactcgaaaaagaagtggggcgctgcgagaatatcgtgtccaaatacgaaacccgctacggcccactc

SEQ ID NO: 116 - protein, 49 aa, Synthetic construct
GSGSGSITIRAAFLEKENTALRTEIAELEKEVGRCENIVSKYETRYGPL

SEQ ID NO: 117 - DNA, 147 bp, Synthetic construct
  source 1..147: mol_type = other DNA
ggtagcggctccggctccattacaattagagccgccttcctggaaaaagagaacacagccctgagaaccgaggtggccgaactcgagaaagaagtgggccgctgcgagaacatcgtgtctaagtacgagacaagatacgggcccctg

SEQ ID NO: 118 - protein, 49 aa, Synthetic construct
GSGSGSITIRAAFLEKENTALRTEVAELEKEVGRCENIVSKYETRYGPL

SEQ ID NO: 119 - DNA, 147 bp, Synthetic construct
  source 1..147: mol_type = other DNA
ggatctggctctggatccattacaatcagagccgccttcctcgagaaagagaacacagccctgagaaccgagaacgccgagctggaaaaagaagtgggccgctgcgagaacatcgtgtctaagtacgagacaagatacggcccactc

SEQ ID NO: 120 - protein, 49 aa, Synthetic construct
GSGSGSITIRAAFLEKENTALRTENAELEKEVGRCENIVSKYETRYGPL

SEQ ID NO: 121 - DNA, 663 bp, Synthetic construct
  source 1..663: mol_type = other DNA
ggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 122 - protein, 221 aa, Synthetic construct
GSGATNFSLLKQAGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 123 - protein, 27 aa, Synthetic construct
FWVLVVVGGVLACYSLLVTVAFIIFWV

SEQ ID NO: 124 - DNA, 81 bp, Synthetic construct; identical to SEQ ID NO: 103
  source 1..81: mol_type = other DNA

SEQ ID NO: 125 - protein, 46 aa, Synthetic construct
GPAGSLLGSGQMQITLWGSLAAVAIFFVITFLIFLCSSCDREKKPR

SEQ ID NO: 126 - DNA, 138 bp, Synthetic construct
  source 1..138: mol_type = other DNA
ggtccggctggctctctgctcggcagtggtcagatgcagattacgttgtggggcagtttggcagccgtcgcaatcttctttgttatcacttttcttatctttctctgttcctcatgtgacagagagaaaaagccccga

SEQ ID NO: 127 - protein, 36 aa, Synthetic construct
MEADALSPVGLGLLLLPFLVTLLAALCVRCRELPVS

SEQ ID NO: 128 - DNA, 108 bp, Synthetic construct
  source 1..108: mol_type = other DNA
atggaagccgatgctctgtctcctgttggcctgggactgctgctcctgccttttctggttacactgctggccgctctgtgtgtgcggtgtagagaactgccagttagt

SEQ ID NO: 129 - protein, 36 aa, Synthetic construct
MEADALSPVGLGLLLLPFLVTLLAALAVRARELPVS

SEQ ID NO: 130 - DNA, 108 bp, Synthetic construct
  source 1..108: mol_type = other DNA
atggaagccgatgctctgtctcctgttggcctgggactgctgctcctgccttttctcgttacactgctggccgctctggctgtgcgagctagagaactgcctgtgtct

SEQ ID NO: 131 - protein, 32 aa, Synthetic construct
EEAILVPCVLGLLLLPILAMLMALCVHCHRLP

SEQ ID NO: 132 - DNA, 96 bp, Synthetic construct
  source 1..96: mol_type = other DNA
gaggaagcaatcctggtgccgtgtgtacttggtctgcttttgttgccaatacttgcgatgctcatggctctctgcgtacattgccatcggcttccg

SEQ ID NO: 133 - protein, 21 aa, Synthetic construct
IFSGFAGLLAILLVVAVFCIL

SEQ ID NO: 134 - DNA, 63 bp, Synthetic construct
  source 1..63: mol_type = other DNA
atcttcagcggctttgccggactgctggctatcctgctggttgtggccgtgttctgtatcctt

SEQ ID NO: 135 - protein, 21 aa, Synthetic construct
LCYLLDGILFIYGVILTALFL

SEQ ID NO: 136 - DNA, 63 bp, Synthetic construct
  source 1..63: mol_type = other DNA
ctgtgctacctgctggacggcatcctgtttatctacggcgtgatcctgacagccctgttcctt

SEQ ID NO: 137 - protein, 21 aa, Synthetic construct
ALIAFLAFLIIVTSIALLVVL

SEQ ID NO: 138 - DNA, 63 bp, Synthetic construct
  source 1..63: mol_type = other DNA
gccctgattgccttcctggcctttctgatcatcgtgaccagcattgccctgctggtcgtgctg

SEQ ID NO: 139 - protein, 42 aa, Synthetic construct
AAAIEVMYPPPYLDNEKSNGTIIHVKGKHLCPSPLFPGPSKP

SEQ ID NO: 140 - protein, 12 aa, Synthetic construct
ESKYGPPCPSCP

SEQ ID NO: 141 - protein, 12 aa, Synthetic construct
ESKYGPPAPSAP

SEQ ID NO: 142 - protein, 12 aa, Synthetic construct
ESKYGPPCPPCP

SEQ ID NO: 143 - protein, 12 aa, Synthetic construct
EPKSCDKTHTCP

SEQ ID NO: 144 - protein, 86 aa, Synthetic construct
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 145 - protein, 45 aa, Synthetic construct; identical to SEQ ID NO: 102

SEQ ID NO: 146 - protein, 132 aa, Synthetic construct
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 147 - protein, 34 aa, Synthetic construct
ACPTGLYTHSGECCKACNLGEGVAQPCGANQTVC

SEQ ID NO: 148 - protein, 20 aa, Synthetic construct
AVGQDTQEVIVVPHSLPFKV

SEQ ID NO: 149 - skipped

SEQ ID NO: 150 - protein, 6 aa, Synthetic construct
GGSGGS

SEQ ID NO: 151 - protein, 9 aa, Synthetic construct
GGSGGSGGS

SEQ ID NO: 152 - protein, 12 aa, Synthetic construct
GGSGGSGGSGGS

SEQ ID NO: 153 - protein, 15 aa, Synthetic construct
GGSGGSGGSGGSGGS

SEQ ID NO: 154 - protein, 4 aa, Synthetic construct
GGGS

SEQ ID NO: 155 - protein, 8 aa, Synthetic construct
GGGSGGGS

SEQ ID NO: 156 - protein, 12 aa, Synthetic construct
GGGSGGGSGGGS

SEQ ID NO: 157 - protein, 16 aa, Synthetic construct
GGGSGGGSGGGSGGGS

SEQ ID NO: 158 - protein, 20 aa, Synthetic construct
GGGSGGGSGGGSGGGSGGGS

SEQ ID NO: 159 - protein, 5 aa, Synthetic construct
GGGGS

SEQ ID NO: 160 - protein, 10 aa, Synthetic construct
GGGGSGGGGS

SEQ ID NO: 161 - protein, 15 aa, Synthetic construct
GGGGSGGGGSGGGGS

SEQ ID NO: 162 - protein, 20 aa, Synthetic construct
GGGGSGGGGSGGGGSGGGGS

SEQ ID NO: 163 - protein, 25 aa, Synthetic construct
GGGGSGGGGSGGGGSGGGGSGGGGS

SEQ ID NO: 164 - protein, 5 aa, Synthetic construct
GGSGS

SEQ ID NO: 165 - protein, 6 aa, Synthetic construct
GGCAGC

SEQ ID NO: 166 - protein, 18 aa, Synthetic construct
GSTSGSGKPGSGEGSTKG

SEQ ID NO: 167 - protein, 20 aa, Synthetic construct
EAAAKEAAAKEAAAKEAAAK

SEQ ID NO: 168 - DNA, 31 bp, Synthetic construct
  source 1..31: mol_type = other DNA
agagggtatataatggaagctcgacttccag

SEQ ID NO: 169 - DNA, 52 bp, Synthetic construct
  source 1..52: mol_type = other DNA
gggaatttccggggactttccgggaatttccggggactttccgggaatttcc

SEQ ID NO: 170 - DNA, 85 bp, Synthetic construct
  source 1..85: mol_type = other DNA
caccagacagtgacgtcagctgccagatcccatggccgtcatactgtgacgtctttcagacaccccattgacgtcaatgggagaa

SEQ ID NO: 171 - DNA, 90 bp, Synthetic construct
  source 1..90: mol_type = other DNA
ggaggaaaaactgtttcatacagaaggcgtggaggaaaaactgtttcatacagaaggcgtggaggaaaaactgtttcatacagaaggcgt

SEQ ID NO: 172 - DNA, 115 bp, Synthetic construct
  source 1..115: mol_type = other DNA
aggatgtccatattaggacatctaggatgtccatattaggacatctaggatgtccatattaggacatctaggatgtccatattaggacatctaggatgtccatattaggacatct

SEQ ID NO: 173 - DNA, 115 bp, Synthetic construct
  source 1..115: mol_type = other DNA
agtatgtccatattaggacatctaccatgtccatattaggacatctactatgtccatattaggacatcttgtatgtccatattaggacatctaaaatgtccatattaggacatct

SEQ ID NO: 174 - DNA, 48 bp, Synthetic construct
  source 1..48: mol_type = other DNA
tgagtcagtgactcagtgagtcagtgactcagtgagtcagtgactcag

SEQ ID NO: 175 - DNA, 120 bp, Synthetic construct
  source 1..120: mol_type = other DNA
agatcaaagggtttaagatcaaagggcttaagatcaaagggtataagatcaaagggcctaagatcaaagggactaagatcaaagggtttaagatcaaagggcttaagatcaaagggccta

SEQ ID NO: 176 - DNA, 32 bp, Synthetic construct
  source 1..32: mol_type = other DNA
gtctagacgtctagacgtctagacgtctagac

SEQ ID NO: 177 - DNA, 24 bp, Synthetic construct
  source 1..24: mol_type = other DNA
cagacacagacacagacacagaca

SEQ ID NO: 178 - DNA, 65 bp, Synthetic construct
  source 1..65: mol_type = other DNA
ggatccggtactcgagatctgcgatctaagtaagcttggcattccggtactgttggtaaagccac

SEQ ID NO: 179 - DNA, 66 bp, Synthetic construct
  source 1..66: mol_type = other DNA
gggactttccactggggactttccactggggactttccactggggactttccactggggactttcc

SEQ ID NO: 180 - DNA, 51 bp, Synthetic construct
  source 1..51: mol_type = other DNA
agacgctagcggggggctataaaagggggtgggggcgttcgtcctcactct

SEQ ID NO: 181 - DNA, 140 bp, Synthetic construct
  source 1..140: mol_type = other DNA
gggactttccactggggactttccactggggactttccactggggactttccactggggactttccactcctgcaggagctggcgcgccagacgctagcggggggctataaaagggggtgggggcgttcgtcctcactct

SEQ ID NO: 182 - DNA, 25 bp, Synthetic construct
  source 1..25: mol_type = other DNA
tctagagggtatataatgggggcca